(19) United States
(12) Patent Application Publication
(10) Pub. No.: US 2026/0258113 A1
(43) Pub. Date: Sep. 3, 2026

(54) BETA GLOBIN MIMETIC PEPTIDES AND THEIR USE

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Hans Christian Ackerman, Rockville, MD (US); Steven David Brooks, Morgantown, WV (US); Phillip Cruz, Clarksburg, MD (US); Rolf Eric Swenson, Silver Spring, MD (US)

(73) Assignee: THE U.S.A., as represented by the Secretary, Department of Health and Human Services, Bethesda, MD (US)

(21) Appl. No.: 18/854,503

(22) PCT Filed: Apr. 6, 2023
(86) PCT No.: PCT/US2023/065432

§ 371 Date: Oct. 4, 2024

Related U.S. Application Data
(60) Provisional application No. 63/328,615, filed on Apr. 7, 2022.

(51) Int. Cl. C07K 14/805; A61K 38/00

(52) U.S. Cl. CPC C07K 14/805; A61K 38/00; C07K 2319/10

(57) ABSTRACT

Disclosed is an isolated polypeptide comprising a Hbb peptide, wherein the Hbb peptide comprises one of: a) X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid; b) HFX₇KEX₈X₉PPV (SEQ ID NO: 2), wherein X₇ is any amino acid, X₈ is any amino acid, and X₉ is T or S; or c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁ₛ is any amino acid, wherein the isolated peptide is at most 75 amino acids in length. Molecules including these isolated polypeptides are disclosed, as well as nucleic acid molecules and vectors encoding these polypeptides. Pharmaceutical compositions including the polypeptides, molecules, nucleic acids and vectors are of use for increasing vascular nitric oxide bioavailability and/or inhibiting vasoconstriction in a subject.

### CROSS REFERENCE TO RELATED APPLICATIONS

This claims the benefit of U.S. Provisional Application No. 63/328,615, filed Apr. 7, 2022, which is incorporated herein by reference.

### STATEMENT OF GOVERNMENT SUPPORT

This invention was made with Government support under project number Z01 #: AI001150 by the National Institutes of Health, National Institute of Allergy and Infectious Diseases. The United States Government has certain rights in the invention.

### FIELD OF THE DISCLOSURE

This relates to the field of the vasoreactivity and nitric oxide synthesis in arteries, and specifically to methods for inhibiting vasoconstriction in a subject.

### REFERENCE TO AN ELECTRONIC SEQUENCE LISTING

The contents of the electronic sequence listing (4239-108139-02 Sequence Listing.xml; Size: 51,051 bytes; and Date of Creation: Mar. 20, 2023) is herein incorporated by reference in its entirety.

### BACKGROUND

Peripheral vascular resistance is primarily provided by small arteries of the vascular system that link conduit arteries to arterioles feeding capillary beds. Peripheral small arteries can constrict to increase blood pressure or redistribute blood flow to meet regional demand. Third and fourth order arteries that perfuse the visceral organs are especially dynamic in their vasoreactivity. These arteries can dilate to provide increased blood flow during digestion² and constrict to divert blood flow away from visceral organs to maintain cerebral pressure or to support skeletal muscle during physical activity. The signals that mediate vasoreactivity in small mesenteric arteries can act through vascular endothelial cells or directly on vascular smooth muscle cells through nervous innervation.

Alpha-adrenergic signaling plays a major role in the regulation of systemic blood pressure by directly stimulating vascular smooth muscle cells to constrict. Activation of alpha-1-adrenergic receptors triggers vascular smooth muscle cells (vSMCs) to constrict by activating PL-C and increasing IP3, leading to increased intracellular calcium release and subsequent activation of myosin light chain kinase. This vasoconstrictive stimulus is counterbalanced by a vasodilatory signal emanating from the adjacent endothelial cell, which produces both nitric oxide (NO) and endothelial-derived hyperpolarizing factor (EDHF) in response to adrenergic constriction of vSMCs. The depolarization of vSMCs during adrenergic stimulation opens L-type voltage gated calcium channels on their membrane, near the myoendothelial junctions (MEJs) that connect vSMCs with endothelial cells. The local influx of calcium near the MEJ within the vSMC is then communicated to the endothelial cell via both direct and indirect calcium and IP₃ signaling. This signaling from the vSMC to the endothelium induces intracellular release of calcium within the endothelium to produce vasodilatory stimuli to relax the vascular smooth muscle back towards its resting state. These vasodilatory stimuli include the efflux of K⁺ ions from the endothelial cell that hyperpolarize the vSMC and calcium-dependent activation of endothelial NO synthase (eNOS) via calmodulin to produce NO, a potent vasodilatory molecule. Thus, alpha-1-adrenergic vasoconstriction induces a counterbalancing vasodilation signal from endothelium, a mechanism referred to as feedback vasodilation.

Feedback vasodilation by endothelium-derived nitric oxide is itself under the regulation of globins. Originally, alpha globin was identified in mouse thoracodorsal arteries as a key globin that bound to eNOS in the MEJ to restrict the release of NO from eNOS. Both the proximity of alpha globin to eNOS and the oxidative state of the alpha globin heme determine the extent to which NO release can be restricted. In our studies of this pathway in human resistance arteries, it was discovered that in humans, unlike in mice, both the alpha globin and the beta globin subunits of hemoglobin are expressed in the artery wall. The structures of tetrameric hemoglobin bound to eNOS were modeled, and it was predicted which sequences of the newly identified beta globin chain would be most critical for stabilizing the interactions with eNOS. Mimetic peptides were designed based on these predictions, linked them with cell penetrating peptides, and tested them for their ability to enhance feedback vasodilation in human arteries that are exposed to the alpha-1-adrenergic receptor agonist phenylephrine. These peptides disrupt the binding of beta globin to eNOS, diminish the ability of beta globin to restrict NO release, and thereby enhance feedback vasodilation. These agents can be used to increase NO signaling from endothelial cells and thus inhibit, prevent, or reverse vasoconstriction. A need remains for agents that affect eNOS, and alter vasoconstriction.

### SUMMARY OF THE DISCLOSURE

Disclosed is an isolated polypeptide comprising a Hbb peptide, wherein the Hbb peptide comprises one of: a) X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid; b) HFX₇KEX₈X₉PPV (SEQ ID NO: 2), wherein X₇ is any amino acid, X₈ is any amino acid, and X₉ is T or S; or c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁₅ is any amino acid, wherein the isolated peptide is at most 75 amino acids in length. Optionally the isolated peptide can include a cell penetrating peptide.

In further embodiments, conjugates including these isolated peptides are disclosed that include the isolated polypeptide and an HbαX peptide.

In more embodiments, disclosed are agents, such as fusion proteins, that include the isolated polypeptide or the conjugate and a second Hbb peptide In yet other embodiments, nucleic acid molecules and vectors are disclosed encoding these polypeptides and/or Hbb peptides.

Pharmaceutical compositions are disclosed that include the polypeptides, Hbb peptides, conjugates, fusion proteins, nucleic acids and/or vectors. These pharmaceutical compositions are of use for increasing vascular nitric oxide bioavailability and/or inhibiting vasoconstriction in a subject.

The foregoing and other features and advantages of the invention will become more apparent from the following detailed description of several embodiments which proceeds with reference to the accompanying figures.

### BRIEF DESCRIPTION OF THE FIGURES

FIGS. 1A-1C: Gene expression in perfused human small arteries dissected from omental tissue (n=8); data is plotted with geometric mean. A) Total copies of HBA1, HBA2, HBB, NOS3, SLC4A1 and a no-reverse transcriptase reaction control, per 1 ng of cDNA. B) The expression ratio of the erythrocyte specific marker SLC4A1 to HBA1 is lower in omental arteries (n=8) compared to human whole blood (n=3)(p=0.016), and C) the expression ratio HBA1 to HBA2 is lower in omental arteries (n=8) compared to human whole blood (n=3)(p=0.010).

FIGS. 2A-2D: A) Beta globin, eNOS, and cytochrome B5 reductase 3 (Cyb5R3) co-immunoprecipitate with alpha globin in lysates of perfused human omental arteries. P1, P2, and P3 each represent an independent replicate consisting of pooled flash-frozen arteries from two donors (n=30 arteries from each donor). P1 and P2 were incubated with an alpha globin antibody to immunoprecipitate alpha globin and bound protein partners. P3 was incubated instead with an IgG antibody as a negative control. Western blot was performed on the immunoprecipitate with antibodies for alpha globin, beta globin, Cyb5R3, or eNOS. Myc-eNOS purified from a cell overexpression lysate served as a positive control for eNOS. RBC lysate served as a positive control for alpha and beta globin. B) The red blood cell protein Band 3/SLC4A1 is not detected in protein lysates from pooled human omental arteries. Prior to immunoprecipitation, whole protein lysate was loaded into a gel and probed by Western Blot for SLC4A1 and alpha globin. Gel exposure was maximized to probe for any SLC4A1 signal in pooled samples P1 or P2; while alpha globin was abundant in both lysates, no SLC4A1 was detected, indicating that arteries were fully perfused free of RBCs. C-D) Biolayer interferometry sensorgram traces of the binding of eNOS or AHSP to alpha globin showing association and dissociation; raw datasets shown in black, fitting curves shown. C) Binding between Alpha-globin and eNOSₒₓ. The sensors were dipped in 200 mL of eNOSₒₓ solution at 4000 nM, 2000 nM, 1000 nM, 500 nM, 250 nM for 120 seconds for association, and then moved to assay buffer for another 120 seconds for dissociation. D) Binding between Alpha-globin and AHSP: The sensors were dipped in 200 mL of AHSP solution at 1000 nM, 500 nM, 250 nM, 125 nM, 62.5 nM for 120 seconds for association, and then moved to assay buffer for another 120 seconds for dissociation.

FIGS. 3A-3D: Hemoglobin presents as autofluorescent punctates in the walls of omental arteries. A) Longitudinal view of intact omental artery viewed from a 1 μm Z-step in plane with the arterial endothelium and internal elastic lamina (IEL, green). The hemoglobin-eNOS complexes punctates) are broadly autofluorescent. Endothelial cell nuclei (DAPI) are aligned in the direction of flow within the vessel. The outer collagen layer demonstrates second harmonic generation (SHG, blue). B) Longitudinal view of intact omental artery viewed from a 1 μm Z-step in plane with the arterial endothelium and internal elastic lamina (IEL) in a non-perfused artery; residual RBCs (bright pink) demonstrate broad autofluorescence. C) Longitudinal view of intact perfused murine mesenteric artery viewed from a 1 μm Z-step in plane with the arterial endothelium and the IEL; endothelial nuclei are visible. D) View of non-perfused intact murine mesenteric artery; residual RBCs demonstrate broad autofluorescence.

FIGS. 4A-4K: Fluorescence Lifetime Imaging Microscopy (FLIM) of intact omental arteries shows specific binding of antibodies at the autofluorescent punctate. Arteries were split, with one half imaged unstained and the other half labeled with a specific antibody. Total number of fluorescent lifetime events were plotted for the unstained and antibody labeled sections for A) alpha globin, D) beta globin, and G) eNOS. To focus on fluorescent lifetime differences within the distinct autofluorescent punctates with and without antibody labeling, Regions of Interest (ROIs) were selected for 10 of the autofluorescent antibody complexes from each labeling condition (B, E, I); the lifetime pseudocolor lookup table encodes lower lifetimes as blue and longer lifetimes as green or red. The mean fluorescent lifetime within each ROI was then calculated and compared to unstained control (C, F, I). Mean fluorescence was significantly decreased following labeling with alpha globin (C), beta globin (F), or eNOS (I) (line shown at geometric mean; p<0.0001 for all comparisons). J) FLIM was then performed on an intact omental artery as unstained, labeled with alpha globin antibody only, labeled with eNOS antibody only, or co-labeled with antibodies for alpha globin and eNOS. Co-labeling with eNOS and alpha globin antibodies shifted the peak lifetime further left of alpha globin or eNOS alone, indicating that the two antibodies were generating a possible FRET-like effect due to spatial proximity. K) ROIs were selected for each labeling condition as mean fluorescent lifetimes calculated for each ROI. Alpha globin antibody and eNOS antibody each decreased mean lifetime compared to unstained control (p<0.0001); co-labeling with both eNOS and alpha globin antibodies further decreased the mean fluorescence lifetime compared to single-stained arteries (p<0.0001).

FIGS. 5A-5E: Multiphoton imaging of intact omental arteries. A) Unlabeled intact omental artery. The collagen of the tunica externa demonstrates second harmonic generation signal (SHG) in the blue part of the spectrum; the elastin of the internal elastic lamina (IEL) is autofluorescent in the green and red spectra. B) Transverse cross section, 3 μm in the Z-plane, of intact small omental artery shown in panel A, labeled with DAPI to demarcate nuclei. The transverse section shows localization of autofluorescent hemoglobin-eNOS complexes, which fluoresce broadly to the IEL. C) Longitudinal cross section, taken as 1 μm thick Z-step image of omental artery at the midline shows, the endothelial cell nuclei aligned in parallel with the direction of the artery. The autofluorescent complexes are in the same 1 μm plane as endothelial nuclei and the IEL. D) Three-dimensional image of segment from intact omental artery in panel A, viewed in the transverse plane and extending for 40 μm. Endothelial nuclei are visible interior to the IEL, while smooth muscle nuclei are visible between the IEL and the collagen of the tunica externa. The autofluorescent complexes are localized to the IEL. E) Image deconvolution of omental artery, opened and imaged enface, as a three-dimensional surface model using Imaris Bitplane. Hemoglobin-eNOS complexes are visualized, and are embedded within, and protruding through, the IEL (yellow). The collagen of the tunica externa is visible.

FIGS. 6A-6B: Change in inner diameter (as percentage of resting baseline) of omental arteries to phenylephrine (PE) with four-parameter logistic regression models fit to the data. Responses are plotted as (mean diameter, SE). Responses to only PE were not different between the two groups (p=0.66). A) Responses of omental arteries to PE, PE after incubation with an alpha globin mimetic peptide, and PE after incubation with the mimetic peptide and L-NAME. Treatment with the mimetic peptide significantly inhibited the vasoconstriction response to PE (p<0.0001) but in the presence of the NOS inhibitor L-NAME, vasoconstriction was restored (p<0.0001). B) Responses of omental arteries to PE, PE after incubation with a control peptide, and PE after incubation with the control peptide and L-NAME. There was no significant difference between response to PE following treatment with the control peptide (p=0.92) or the peptide and L-NAME (p=0.99). The response of the arteries at each dose of PE was analyzed by 2-way Repeated Measures ANOVA.

FIG. 7. Schematic representation of the targeting endothelial hemoglobin to increase NO release from the endothelial cell, within the context of other therapeutic approaches.

FIG. 8. Model of hemoglobin bound to eNOS. The 3 mimetic peptides are labeled.

FIGS. 9A-9C. Close up view of the interactions between Hbb1 and eNOS from FIG. 8. Amino acids on both Hbb1 and eNOS that are involved in binding interactions are labeled and their sidechains shown.

FIG. 10. Close up view of the interactions between Hbb2 and eNOS from FIG. 8. Amino acids on both Hbb2 and eNOS that are involved in binding interactions are labeled and their sidechains shown.

FIG. 11. Close up view of the interactions between Hbb3 and eNOS from FIG. 8. Amino acids on both Hbb3 and eNOS that are involved in binding interactions are labeled and their sidechains shown.

FIG. 12A-12C. Three different beta globin mimetic peptides inhibit human arterial vasoconstriction responses to phenylephrine (PE) in a NOS-dependent fashion. Vasoconstriction to escalating doses of PE was assessed in isolated human omental arteries (circles). Then, PE response was measured after incubation with each beta globin mimetic peptide (squares). Lastly, the PE response was measured again after incubation with the NOS inhibitor L-NAME (triangles).

FIGS. 13A-13C. Gene expression in perfused human small arteries dissected from subcutaneous adipose tissue. A) Total copies of HBA1, HBA2, HBB, NOS3, SLC4A1 and a no-reverse transcriptase reaction control, per 1 ng of cDNA. B) The expression ratio of the erythrocyte specific marker SLC4A1 to HBA1 is lower in subcutaneous arteries (n=8) compared to human whole blood (n=3)(p=0.024), and C) the expression ratio HBA1 to HBA2 is lower in subcutaneous arteries (n=8) compared to human whole blood (n=3)(p=0.023).

FIGS. 14A-14F. A) Longitudinal view of unlabeled intact omental artery 1. Collagen is autofluorescence visible; IEL is also shown; autofluorescent hemoglobin-eNOS complex can also be detected. B) Globin-eNOS complexes from entire intact omental artery 1 image are converted into individual surface objects in Imaris (n=2388). C) Longitudinal view of unlabeled intact omental artery 2, and D) Imaris conversion of globin-eNOS complexes to surface objects (n=3969). E) The mean fluorescence intensity in each channel is plotted for each surface object from omental artery 1; F) mean fluorescence intensity for surface objects from omental artery 2.

FIGS. 15A-15H. Panels A-D show the size and mean fluorescence intensity of hemoglobin-eNOS complexes and RBCs in the same intact omental artery. A) Artery image was enhanced in Imaris using size and spatial location filters to separate the RBCs from the hemoglobin-eNOS complexes. B) Distinct surface objects were created for each complex and each ROI. C) Zoomed view of surfaces shows delineating of objects encoded for RBCs vs hemoglobin-eNOS complexes. D) Mean fluorescence intensity plotted by mean volume for all hemoglobin-eNOS complexes compared to all RBCs. The autofluorescent hemoglobin-eNOS punctates have higher mean fluorescence intensity and smaller volume compared to RBCs within a non-perfused, intact omental artery. Distinct surface objects were created as ROIs for each of the autofluorescent hemoglobin-eNOS complexes (n=2257) and separately the resident RBCs (n=142) using Imaris Bitplane (FIG. 15A-15C). The autofluorescence of the punctates and RBCs within the range of each HyD detector are shown for the artery pictured in FIG. 2B. Panels E-H illustrate that the autofluorescent hemoglobin-eNOS punctates have higher mean fluorescence intensity and smaller volume compared to RBCs within a non-perfused, intact omental artery. E) Channel 1 detected autofluorescence from 393 nm-472 nm; F) Channel 2 detected autofluorescence from 496 nm-547 nm, G) Channel 3 detected autofluorescence from 590 nm-621 nm, H) Channel 4 detected autofluorescence from 658 nm-717 nm. The volume (voxels) and mean fluorescence intensity of each punctate and each RBC was calculated using Imaris (Bitplane) for each channel. The hemoglobin-eNOS complexes (median, 95% CI) [118 voxels, (113, 126)] were significantly smaller than the RBCs (p=0.0001) [1323 voxels, (1042-1538)]. Mean fluorescence intensity was lowest in Channel 1, but the intensities for the punctates and RBCs were clearly distinct in each channel, with punctates having higher mean intensities in all four channels (p=0.0001 for each).

FIGS. 16A-16G. Panels A-D are representative schematic for calculating the ratio of hemoglobin-eNOS complexes to endothelial nuclei in an intact omental artery. A) A region of interest (ROI) is identified with in an image of an intact omental artery. B) The ROI is cropped to size, then C) the elastin autofluorescence is digitally subtracted to leave the autofluorescent complexes and DAPI-stained nuclei. The plane was then adjusted to exclude vascular smooth muscle nuclei (perpendicular alignment), and endothelial nuclei and complexes were counted in ImageJ. D) The total counts for three ROI from a single artery are then averaged to give a single ratio of complexes/nuclei. Panels E-H show spatial density, fluorescence intensity (Channel 4), and per nuclei ratio of hemoglobin-eNOS complexes in intact omental arteries. E). To measure heterogeneity between arteries the density of punctates per analyzed surface area was calculated from seven donor arteries and found a consistent density of punctates. F) To measure heterogeneity between arteries from a single donor, and between donors, the mean fluorescence intensity in channel 4 of hemoglobin-eNOS complexes from five total arteries (two from Donor 1, two from Donor 2, and one from Donor 3) were calculated. Only the artery from Donor 3 was different and was slightly lower in mean channel 4 intensity. G) The ratio of nuclei to globin complexes was calculated as described in FIGS. 17A-17B.

FIGS. 17A-17B. A) Molecular modeling using UCSF Chimera based on computer docking of alpha globin (ribbon on right of figure) with eNOS oxygenase domain homodimer. The alignment of alpha globin with eNOS was predicted using HADDOCK 2.4. The bright red ribbon denotes the peptide sequence of alpha globin that comprises the HbαX mimetic peptide, and the adjacent residues are shown. For contrast, the predicted alignment of the HbαX peptide sequence with eNOS from Straub et al. 2014, originally published in ATVB, is shown, with the adjacent residues shown. The new model shown here is shifted by several Ångstroms compared to the Straub et al. 2014 model, and allows for greater complimentary binding of full tetrameric hemoglobin with eNOS. B) Close-up view of the model showing alpha globin binding with eNOS; alpha globin has been rendered as a surface to show 3-D protein structure. The heme pocket of alpha globin and the heme pocket of eNOS are both visible, demonstrating their spatial proximity to each other to facilitate diffusion of NO from the eNOS reaction center to the alpha globin heme.

FIGS. 18A-18E. Molecular modeling for alpha globin, tetrameric hemoglobin, Cyb5R3, and eNOS created using UCSF Chimera, based on predictive docking models using HADDOCK 2.4. A) Predicted alignment for the alpha globin subunit of hemoglobin with eNOS homodimer; the HbαX mimetic peptide is shown along with adjacent peptide residues. B) Surface representation of tetrameric hemoglobin aligned with eNOS as depicted in FIG. 8; the second alpha subunit and two beta globin units are shown in alignment with eNOS. Several peptide residues of beta globin are predicted to form stable bonds with residues of eNOS in this model, adding stability to the hemoglobin-eNOS complex. C) The model also accommodates the binding of a second hemoglobin with eNOS and the first hemoglobin tetramer-original hemoglobin is on left, with primary alpha subunit from FIG. 18A, the second alpha globin, and beta globin subunits. The second hemoglobin is on the right, with alpha subunits and beta globin subunits also shown. D) Manual VR-enhanced docking of Cyb5R3 with alpha subunit of hemoglobin and eNOS. E) VR-enhanced close-up of the predicted alignment between eNOS, alpha globin, and Cyb5R3 from the model in FIG. 18D. The FAD pocket of Cyb5R3 is visible, with FAD attached, and is spatially very close to the heme pocket of alpha globin; with heme indicated; the reduction of alpha globin by Cyb5R3 is an important mechanism in this model. Both Cyb5R3 and alpha globin bind with eNOS, with the eNOS heme pocket visible, with heme indicated, in close proximity as well.

FIG. 19. Graph showing that subcutaneous adipose resistance arteries from donors with sickle cell trait (i.e., heterozygous of the sickle cell mutation [HbAS]) are partially resistant to phenylephrine-induced vasoconstriction. FIG. 6A

FIG. 20. Hbb-1 inhibits phenylephrine-induced vasoconstriction in canine arteries.

### SEQUENCES

The nucleic and amino acid sequences listed in the accompanying sequence listing are shown using standard letter abbreviations for nucleotide bases, and one letter code for amino acids. Only one strand of each nucleic acid sequence is shown, but the complementary strand is understood as included by any reference to the displayed strand.

SEQ ID NOs: 1-3 are consensus amino acid sequences for Hbb peptides.

SEQ ID NOs: 4-12 are amino acid sequences of exemplary Hbb peptides.

SEQ ID NO: 13 is the sequence of human beta globin.

SEQ ID NOs: 14-47 are amino acid sequences of exemplary cell penetrating peptides.

SEQ ID NOs: 48-50 are amino acid sequences of exemplary linkers.

SEQ ID NOs: 51 and 52 are amino acid sequences of exemplary Hbα peptides.

### DETAILED DESCRIPTION OF SEVERAL EMBODIMENTS

The release of NO from eNOS across the myoendothelial junction is under a final level of regulation through protein-protein interactions of eNOS with alpha globin. Studies of skeletal muscle resistance arteries from mice demonstrated that alpha globin is found in vascular endothelial cells where it binds to eNOS and regulates the diffusion of nitric oxide across the MEJ during endothelium-dependent vasodilation and in response to alpha-1-adrenergic vasoconstriction (Straub et al., Nature 491:473-477, 2012; Straub et al., Arterioscler Thromb Vasc Biol. 34:2594-2600, 2014). In mice, formation of the alpha globin-eNOS complex requires the presence of alpha hemoglobin stabilizing protein (AHSP) (Lechauve et al., J Clin Invest. 128:5073-5082, 2018). Molecular modeling studies identified a 10 amino acid sequence, conserved across species, that was predicted to interact with eNOS (Straub et al., Arterioscler Thromb Vasc Biol. 34:2594-2600, 2014; Keller et al., Hypertens Dallas Tex 1979 68.6:1494-1503, 2016). This sequence was used to encode an alpha globin mimetic peptide, named HbαX, which was shown to disrupt the association of alpha globin with eNOS in vivo, resulting in increased NO bioavailability, reduced alpha-1-adrenergic vasoconstriction, lower mean arterial blood pressure, and protection against angiotensin- 2 induced hypertension in mice (Straub et al., Arterioscler Thromb Vase Biol. 34:2594-2600, 2014; Keller et al., Hypertens Dallas Tex 1979 68.6:1494-1503, 2016).

The expression, sub-cellular localization, and binding partners of alpha globin was determined in human omental resistance arteries. The HbαX alpha globin mimetic peptide was used to disrupt the association of alpha globin with eNOS in human omental arteries to characterize the functional role of alpha globin in regulating vasoreactivity (Brooks et al., MedRxiv preprint, doi.org/10.1101/2021.04.06.21255004, posted Apr. 9, 2000, incorporated herein by reference). These studies established a role for endothelial alpha globin as a restrictor of nitric oxide diffusion in human omental arteries that directly modulates feedback vasodilation to an alpha-1-adrenergic agonist.

Furthermore, to study the role of other globins in regulating vasoreactivity of human resistance arteries, omental tissue was obtained during abdominal surgical operations. Resistance arteries 100-200 um in diameter were microdissected from the omental tissue, individually cannulated and perfused to remove blood, and then subjected to a range of molecular, biochemical, imaging, and functional studies. This systematic approach, carried out entirely in the context of human arteries, provided compelling data supporting the conclusion that hemoglobin (not alpha globin alone) regulates nitric oxide signaling and vasoreactivity by binding to eNOS in human arteries (Brooks et al., MedRxiv preprint, doi.org/10.1101/2021.04.06.21255004, posted Apr. 9, 2000, incorporated herein by reference).

Furthermore, three regions of the beta globin polypeptide were identified using molecular modeling that interact with and bind to residues on eNOS. These peptides have vasodilatory activity and may act by displacing beta globin from eNOS to enhance the release of nitric oxide and dilate the artery. The peptides disclosed herein can be used as vascular therapeutics that increase endothelial nitric oxide release to counteract a vasoconstrictive stimulus.

### II. Terms and Methods

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context indicates otherwise. For example, the term “a protein” includes single or plural proteins and can be considered equivalent to the phrase “at least one protein.” As used herein, the term “comprises” means “includes.” Unless otherwise indicated “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Animal: Living multi-cellular vertebrate organisms, a category that includes, for example, mammals and birds. The term mammal includes both human and non-human mammals. Similarly, the term subject includes both human and veterinary subjects, for example, humans, non-human primates, dogs, cats, horses, and cows.

Administration: Administration of an active compound or composition can be by any route known to one of skill in the art. Administration can be local or systemic. Examples of local administration include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Amino acid substitution: The replacement of one amino acid in a polypeptide with a different amino acid including unnatural amino acids N-methyl amino acids or D-amino acids can be introduced to increase proteolytic stability.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound. It is acknowledged that these terms may overlap in some circumstances.

Atherosclerosis: The progressive narrowing and hardening of a blood vessel over time. Atherosclerosis is a common form of arteriosclerosis in which deposits of yellowish plaques (atheromas) containing cholesterol, lipoid material and lipophages are formed within the intima and inner media of large and medium-sized arteries. Treatment of atherosclerosis includes reversing or slowing the progression of atherosclerosis, for example as measured by the presence of atherosclerotic lesions and/or functional signs of the disease, such as improvement in cardiovascular function as measured by signs (such as peripheral capillary refill), symptoms (such as chest pain and intermittent claudication), or laboratory evidence (such as that obtained by EKG, angiography, or other imaging techniques).

Aryl: A monovalent or divalent aromatic carbocyclic group of, unless specified otherwise, from 6 to 15 carbon atoms having a single ring (e.g., phenyl) or multiple condensed rings in which at least one ring is aromatic (e.g., quinoline, indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic. The aryl group can be attached to the peptide by coupling to a carboxyl group (with an amine) or amine (with a carboxyl group).

Blood Pressure: The pressure of blood pushing against the walls of the arteries, measure as systolic and diastolic blood pressure. Blood pressure of less than 120/80 mm Hg are considered normal range. Elevated blood pressure is when readings consistently range from 120-129 systolic and less than 80 mm Hg diastolic. People with elevated blood pressure are likely to develop high blood pressure unless steps are taken to control the condition. Hypertension stage 1 is when blood pressure consistently ranges from 130-139 systolic or 80-89 mm Hg diastolic. Hypertension stage 2 is when blood pressure consistently ranges at 140/90 mm Hg or higher. Hypertensive crisis is a blood pressure reading that suddenly exceed 180/120 mm Hg.

Control: A reference standard. In some embodiments, the control is a negative control sample obtained from a healthy subject. In other embodiments, the control is a positive control sample obtained from a subject with a disease condition, or from the same subject prior to treatment. In still other embodiments, the control is a historical control or standard reference value or range of values.

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Cell Penetrating Peptide: Short peptides, generally less than 30 amino acids, that translocate into cells and facilitate attached cargo or fusion peptides to translocate across the plasma membrane. Cell penetrating peptides generally do not disturb the structure of the plasma membrane. CPPsite 2.0 (crdd.osdd.net/raghava/cppsite/) database contains about 1850 kinds of cell penetrating peptide sequences. These peptides can be linear or cyclic. These peptides can be cationic, amphipathic, or hydrophobic. Specific motifs and structures have been associated with the cell penetration function. Cell penetrating peptides are reviewed in Xie et al., Front. Pharmacol. 20 May 2020 | doi.org/10.3389/fphar.2020.00697, and are typically short peptides with an abundance of positively charged amino acids such as Arg and Lys.

Conjugate: A complex of two molecules linked together, for example, linked together by a covalent bond. In one embodiment, an Hbb peptide is linked to an HbαX peptide. The linkage can be by chemical or recombinant means. In one embodiment, the linkage is chemical, wherein a reaction between the Hbb peptide and the HbαX peptide has produced a covalent bond formed between the two molecules to form one molecule. In another embodiment, a heterologous peptide linker (short peptide sequence) is used to link two peptides together as a single fusion protein. In a further embodiment, a chemical linker can be utilized.

Consists Of: A polypeptide of a specified amino acid sequence that does not include any additional amino acid residues. The residues in the polypeptide can be modified to include non-peptide components. The N- and/or C-terminus of a polypeptide that consists of a specified amino acid sequence can be joined (for example, by a covalent bond) to a chemical linker for conjugation chemistry. A polypeptide that consists of a specified amino acid sequence can be glycosylated and/or can include non-naturally occurring amino acids.

Degenerate variant: A polynucleotide encoding a peptide that includes a sequence that is degenerate as a result of the genetic code. There are 20 natural amino acids, most of which are specified by more than one codon. Therefore, all degenerate nucleotide sequences are included in this disclosure as long as the amino acid sequence of the polypeptide encoded by the nucleotide sequence is unchanged.

Effective amount: An amount of agent, such as a peptide, that is sufficient to elicit a desired response, such as reducing vasoconstriction in a subject. It is understood that to obtain an effect, a method can require multiple administrations of a disclosed agent.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Fusion protein: A protein comprising two or more amino acid sequences that are not found joined together in nature.

Hemoglobin (Hb): The iron-containing oxygen-transport metalloprotein in red blood cells of vertebrates and other animals. In humans, the hemoglobin molecule is an assembly of four globular protein subunits. Each subunit is composed of a protein chain tightly associated with a non-protein heme group. Each protein chain arranges into a set of alpha-helix structural segments connected together in a globin fold arrangement, so called because this arrangement is the same folding motif used in other heme/globin proteins. This folding pattern contains a pocket which strongly binds the heme group.

Heterologous: A type of sequence that is not normally (for example, in the wild-type sequence) found adjacent to a second sequence. In one embodiment, the sequence is from a different genetic source, such as a virus or other organism, than the second sequence. Methods for introducing a heterologous nucleic acid molecule in a cell or organism are well known in the art, for example transformation with a nucleic acid, including electroporation, lipofection, particle gun acceleration, and homologous recombination. A heterologous peptide linker includes an amino acid sequence that is not found next to a specified sequence in the wild-type protein, such as a sequence from a different protein or a synthetic peptide sequence.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Inhibiting or treating a disease: Inhibiting the full development of a disease or condition, for example, in a subject who is at risk for vasoconstriction or an associated condition, such as high blood pressure, myocardial infarction, or stroke. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of high blood pressure, stroke or myocardial infarction. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Injectable composition: A pharmaceutically acceptable fluid composition comprising at least one active ingredient, for example, a protein, peptide, or antibody. The active ingredient is usually dissolved or suspended in a physiologically acceptable carrier, and the composition can additionally comprise minor amounts of one or more non-toxic auxiliary substances, such as emulsifying agents, preservatives, pH buffering agents and the like. Such injectable compositions that are useful for use with the compositions of this disclosure are conventional; appropriate formulations are well known in the art.

Ischemia: A vascular phenomenon in which a decrease in the blood supply to a bodily organ, tissue, or part is caused, for instance, by constriction or obstruction of one or more blood vessels. Ischemia sometimes results from vasoconstriction or thrombosis or embolism. Ischemia can lead to direct ischemic injury, tissue damage due to cell death caused by reduced oxygen supply. Ischemia can occur acutely, as during surgery, or from trauma to tissue incurred in accidents, injuries and war settings, for instance. It can also occur sub-acutely, as found in atherosclerotic peripheral vascular disease, where progressive narrowing of blood vessels leads to inadequate blood flow to tissues and organs.

Ischemia/reperfusion injury: In addition to the immediate injury that occurs during deprivation of blood flow, ischemic/reperfusion injury involves tissue injury that occurs after blood flow is restored. Current understanding is that much of this injury is caused by chemical products and free radicals released into the ischemic tissues.

When a tissue is subjected to ischemia, a sequence of chemical events is initiated that may ultimately lead to cellular dysfunction and necrosis. If ischemia is ended by the restoration of blood flow, a second series of injurious events ensue, producing additional injury. Thus, whenever there is a transient decrease or interruption of blood flow in a subject, the resultant injury involves two components—the direct injury occurring during the ischemic interval and the indirect or reperfusion injury that follows. When there is a long duration of ischemia, the direct ischemic damage, resulting from hypoxia, is predominant. For relatively short duration ischemia, the indirect or reperfusion mediated damage becomes increasingly important. In some instances, the injury produced by reperfusion can be more severe than the injury induced by ischemia per se. This pattern of relative contribution of injury from direct and indirect mechanisms has been shown to occur in all organs.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated. An isolated protein can be produced by a synthetic method.

Linker and Linked: A molecule that can be used to link two molecules into one contiguous molecule. Non-limiting examples of linkers include peptide linkers, such as glycine-serine links, and 8-amino-3,6-dioxaoctanoic acid moieties. If a peptide linker is involved, the covalent linkage of the first and second polypeptides can be to the N- and C-termini of the peptide linker. Typically, such linkage is accomplished using molecular biology techniques to genetically manipulate DNA encoding the first polypeptide linked to the second polypeptide by the peptide linker. The peptides and linkers can be prepared by solution or solid phase peptide synthesis using standard fluorenylmethoxyoxycarbonyl (Fmoc) or tert-butoxycarbonyl (Boc) amino acid protecting groups and coupling agents such as diisopropyl carbodiimide (DIC), dicyclohexylcarbodiimide (DCC), or HATU etc.

Mammal: This term includes both human and non-human mammals. Similarly, the term subject includes both human and veterinary subjects, for example, humans, non-human primates, mice, rats, dogs, cats, horses, and cows.

Native protein or sequence: A polypeptide or sequence that has not been modified, for example, by selective mutation. Native protein or native sequence are also referred to as wild-type protein or wild-type sequence.

Nitric Oxide Bioavailability: Nitric oxide (NO) is a multifunctional signaling molecule involved in the maintenance of metabolic and cardiovascular homeostasis. NO is also a potent endogenous vasodilator and enters for the key processes that suppresses the formation vascular lesions. Vascular NO bioavailability indicates the abundance of production and utilization of endothelial NO in organisms, its decrease is related to oxidative stress, lipid infiltration, the expressions of some inflammatory factors and the alteration of vascular tone, which plays an important role in endothelial dysfunction. NO synthesized by endothelial nitric oxide synthase plays a role in the maintenance of vascular tone. “Nitric oxide synthase” is an enzyme that catalyzes conversion of 1-arginine, NADPH and oxygen to citrulline, nitric oxide and NADP+. Nitric oxide synthase catalyzes nitric oxide synthesis in the inner lining cells of blood vessels, as well as in macrophages and nerve cells. The generic nomenclature includes all three known isoforms of NOS designated as eNOS, iNOS and nNOS and alternatively as NOS-I, NOS-II and NOS-III.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Peripheral Vascular Disease (PVD): A condition in which the arteries and/or veins that carry blood to and from the arms, legs, soft tissues and vital organs of the body, including the heart and brain, become narrowed or occluded. This interferes with the normal flow of blood, sometimes causing pain but often causing no readily detectable symptoms. With progression of PVD, significant loss of blood flow to tissue and organs can lead to tissue death, necrosis and organ death.

The most common cause of PVD is atherosclerosis, a gradual process in which cholesterol and scar tissue build up, forming plaques that occlude the blood vessels. In some cases, PVD may be caused by blood clots that lodge in the arteries and restrict blood flow.

PVD affects about one in 20 people over the age of 50, or 8 million people in the United States. More than half the people with PVD experience leg pain, numbness or other symptoms, but many people dismiss these signs as a normal part of aging and do not seek medical help.

The most common symptom of PVD is painful cramping in the leg or hip, particularly when walking. This symptom, also known as claudication, occurs when there is not enough blood flowing to the leg muscles during exercise, such that ischemia occurs. The pain typically goes away when the muscles are rested.

Other symptoms may include numbness, tingling or weakness in the leg. In severe cases, people with PVD may experience a burning or aching pain in an extremity such as the foot or toes while resting, or may develop a sore on the leg or foot that does not heal. People with PVD also may experience a cooling or color change in the skin of the legs or feet, or loss of hair on the legs. In extreme cases, untreated PVD can lead to gangrene, a serious condition that may require amputation of a leg, foot or toes. People with PVD are also at higher risk for heart disease and stroke.

Typically most symptomatic PVD is ascribed to peripheral artery disease (PAD) denoting the above described pathology predominantly in arteries. The term PVD includes this symptomology and pathology in all classes of blood vessels.

Pharmaceutical agent: A chemical compound or composition capable of inducing a desired therapeutic or prophylactic effect when properly administered to a subject or a cell. Incubating includes exposing a target to an agent for a sufficient period of time for the agent to interact with a cell. Contacting includes incubating an agent in solid or in liquid form with a cell.

Peptide or Polypeptide: A polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to those that are recombinantly or synthetically produced. A peptide has an amino (N) terminus and a carboxy (C) terminus. The N- or C-terminus of a polypeptide can be joined (for example, by peptide bond) to heterologous amino acids, such as a peptide tag, or a cysteine (or other) residue in the context of a linker for conjugation chemistry.

The phrase “functional fragment(s) of a polypeptide” refers to all fragments of a polypeptide that retain an activity, or a measurable portion of an activity, of the polypeptide from which the fragment is derived. Fragments, for example, can vary in size from a polypeptide fragment as small as an epitope capable of binding an antibody molecule to a large polypeptide capable of participating in the characteristic induction or programming of phenotypic changes within a cell.

Conservative amino acid substitutions are those substitutions that, when made, least interfere with the properties of the original protein, that is, the structure and especially the function of the protein is conserved and not significantly changed by such substitutions. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (T), Leucine (L), Methionine (M), Valine (V); and
6) Phenylalanine (F), Tyrosine (Y), Tryptophan (W).

Conservative substitutions generally maintain (a) the secondary structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartic acid; or (d) a residue having a bulky side chain, for example, phenylalanine, is substituted for (or by) one not having a side chain, for example, glycine.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids and proteins that have been altered by addition, substitution, or deletion of a portion of a natural nucleic acid molecule or protein.

Reperfusion: Restoration of blood supply to tissue that is ischemic, due to decrease in blood supply. Reperfusion is a procedure for treating infarction or other ischemia, by enabling viable ischemic tissue to recover, thus limiting further necrosis. However, reperfusion can itself further damage the ischemic tissue, causing reperfusion injury.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Homologs and variants of a polypeptide (such as an Hbb peptide) are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” or similar language refers to “at least 90%, at least 91%, at least 92%, at least 93%, at least 94%, at least 95%, at least 96%, at least 97%, at least 98%, at least 99%, or even 100% identity” to a specified reference sequence.

Treating a disease: Includes inhibiting or preventing the partial or full development or progression of a disease, for example in a person who is known to have a predisposition to a disease. Furthermore, treating a disease refers to a therapeutic intervention that ameliorates at least one sign or symptom of a disease or pathological condition, or interferes with a pathophysiological process, after the disease or pathological condition has begun to develop.

Under conditions sufficient for: A phrase that is used to describe any environment that permits the desired activity. In one example, includes administering a therapeutically effective amount of a composition that includes a polypeptide as provided herein sufficient to enable the desired activity.

Vasculopathy: A disease of the blood vessels. An “age-related vasculopathy” is a disease of the blood vessels that is associated with advanced age. One specific, non-limiting vasculopathy is atherosclerosis. Other vasculopathies include, but are not limited to, diabetic associated vasculopathy, hypertension associated vasculopathy, Burger's disease associated vasculopathy and scleroderma associated vasculopathy. It is understood that “endothelial dysfunction” typically refers to an insufficiency in the production or response to nitric oxide.

Vasoconstriction. The diminution of the caliber or cross-sectional area of a blood vessel, for instance constriction of arterioles leading to decreased blood flow to a body part. This can be caused by a specific vasoconstrictor, an agent (for instance a chemical or biochemical compound) that causes, directly or indirectly, constriction of blood vessels. α1 adrenoreceptor is a cellular receptor that mediates vasoconstriction when activated by a vasoconstrictor such as phenylephrine or norepinephrine. A vasoconstrictive agent can also be referred to as a vasohypertonic agent, and is said to have vasoconstrictive activity. A representative category of vasoconstrictors is the vasopressor (from the term pressor, tending to increase blood pressure), which term is generally used to refer to an agent that stimulates contraction of the muscular tissue of the capillaries and arteries.

Vasoconstriction also can be due to vasospasm, inadequate vasodilatation, thickening of the vessel wall, or the accumulation of flow-restricting materials on the internal wall surfaces or within the wall itself. Vasoconstriction is a major presumptive or proven factor in aging and in various clinical conditions including progressive generalized atherogenesis, myocardial infarction, stroke, hypertension, glaucoma, macular degeneration, migraine, hypertension and diabetes mellitus, among others.

Vasculature: The network of blood vessels connecting the heart with all other organs and tissues in the body. It includes the arteries and arterioles, bringing oxygen-rich blood to the organs and tissues, and the veins and venules carrying deoxygenated blood back to the heart. A “resistance artery” is a blood vessel in the microcirculation that contributes to the creation of resistance to blood flow. Resistance vessels are innervated by autonomic nerves, and constrict and dilate in response to circulating hormones. Resistance in small arteries (lumen diameter <350 micrometers) and arterioles (lumen diameter <100 micrometers) accounts for 45-50% of total peripheral resistance.

Vasodilation. A state of increased caliber of the blood vessels, or the act of dilation of a blood vessel, for instance dilation of arterioles leading to increased blood flow to a body part. This can be caused by a specific vasodilator, an agent (for instance, a chemical or biochemical compound) that causes, directly or indirectly, dilation of blood vessels. Such an agent can also be referred to as a vasohypotonic agent, and is said to have vasodilative activity. See U.S. Pat. No. 10,370,439.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Unless otherwise explained, all technical and scientific terms used herein have the same meaning as commonly understood by one of ordinary skill in the art to which this invention belongs.

### I. Polypeptides, Conjugates, and Agents

Disclosed herein are polypeptides that include an Hbb peptide, or that consist of the Hbb peptide. An Hbb peptide competes with the binding of beta globin to endothelial nitric oxide synthase, and increases vascular nitric oxide bioavailability. This activity is unique to the disclosed Hbb peptide and peptides including the Hbb peptides.

An Hbb peptide is:
a) X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid;
b) HFX₇KEX₈X₉PPV (SEQ ID NO: 2), wherein X₇ is any amino acid, X₈ is any amino acid, and X₉ is T or S; or
c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁₅ is any amino acid.

The Hbb peptide can include, or consist of, TPEEKSAVTALWGK (SEQ ID NO: 4) (hbb1). The Hbb peptide can include, or consist of, HFGKEFTPPV (SEQ ID NO: 5). The Hbb peptide can include, or consist of, TLSELHCDKLH (SEQ ID NO:6).

In some embodiments, the Hbb peptide includes, or consists of, X₁PEEX₂SAX₃X₄AX₅WX₆K, (SEQ ID NO: 1, wherein X₁ is T or S, X₂ is K or R, X₃ is a hydrophobic amino acid, X₄ is T, S, or a hydrophobic amino acid, X₅ is a hydrophobic amino acid, and X₆ is any amino acid. The Hbb peptide can include, or consist of, TPEEKSALTALWGK (SEQ ID NO: 7). The Hbb peptide can include, or consist of, TPEEKSAVTALWLK (SEQ ID NO: 8).

In some embodiments, W is replaced by 2-naphthyl-alanine or 1-naphthyl-alanine. Polypeptides that are, for example, at least 90% identical to these peptides are also of use. Thus, in some embodiments, the Hbb peptide includes at most 1, or at most two, conservative substitutions in SEQ ID NO: 4. Thus, in more embodiments, the Hbb peptide includes at most 1, or at most two, conservative substitutions in SEQ ID NO: 5. Thus, in other embodiments, the Hbb peptide includes at most 1, or at most two, conservative substitutions in SEQ ID NO: 6. In more embodiments, the Hbb peptide includes at most 1, or at most two, conservative substitutions in SEQ ID NO: 7. In further embodiments, the Hbb peptide includes at most 1, or at most two, conservative substitutions in SEQ ID NO: 8. In some embodiments, W is replaced by 2-naphthyl-alanine or 1-naphthyl-alanine in SEQ ID NOs: 4, 5, 6, 7 or 8.

In some non-limiting examples, the Hbb peptide consists of SEQ ID NO: 4. In other non-limiting examples, the Hbb peptide consists of SEQ ID NO: 5. In further non-limiting examples, the Hbb peptide consists of SEQ ID NO: 6. In more non-limiting examples, the Hbb peptide consists of SEQ ID NO: 7. In some non-limiting examples, the Hbb peptide consists of SEQ ID NO: 8.

In other embodiments, the Hbb peptide includes, or consists of HFX₇KEX₈X₉PPV (SEQ ID NO: 2), wherein X₇ is any amino acid, X₈ is any amino acid, and X₉ is T or S. The Hbb peptide can include, or consist of, HFGKEFTPPV (SEQ ID NO: 5) (hbb2). The Hbb peptide can include, or consist of, HFGKEKTPPV (SEQ ID NO: 9). The Hbb peptide can include, or consist of, HFGKEFLPPV (SEQ ID NO: 10). Polypeptides that are, for example, at least 90% identical to these peptides are also of use. Thus, in some embodiments, the Hbb peptide includes at most 1, or at most two, conservative substitutions in SEQ ID NO: 9. Thus, in more embodiments, the Hbb peptide includes at most 1, or at most two, conservative substitutions in SEQ ID NO: 10.

In more embodiments, the Hbb peptide includes, or consists of, TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar uncharged amino acid, X₁₄ is C, M or T, and X₁₅ is any amino acid. The Hbb peptide can include, or consist of, TLSELYCDKLH (SEQ ID NO: 11). The Hbb peptide can include, or consist of, TLSELHCDKVH (SEQ ID NO: 12). Polypeptides that are, for example, at least 90% identical to these peptides are also of use. Thus, in some embodiments, the Hbb peptide includes at most 1, or at most two, conservative substitutions in SEQ ID NO: 11. Thus, in more embodiments, the Hbb peptide includes at most 1, or at most two, conservative substitutions in SEQ ID NO: 12.

The Hbb peptide can be, for example, 10, 11, 12, 13, 14, 15, 16, 17, 18, 19, 20, 21, 22, 25, 24, or 25 amino acids in length. In some embodiments, the Hbb peptide does not include more than 14 consecutive amino acids of human beta-globin, with or without the N-terminal methionine:

(SEQ ID NO: 13)
(M)VHLTPEEKS AVTALWGKVN VDEVGGEALG RLLVVYPWTQ RFFESFGDLS
TPDAVMGNPK VKAHGKKVLG AFSDGLAHLD NLKGTFATLS ELHCDKLHVD
PENFRLLGNV LVCVLAHHFG KEFTPPVQAA YQKVVAGVAN ALAHKYH.

In further embodiments, the Hbb peptide can include no more than 14, 11 or 10 consecutive amino acids of SEQ ID NO: 13. In some embodiments, the polypeptide consists of the Hbb peptide, and thus is 10, 11 or 14 amino acids in length.

In further embodiments, the polypeptide includes no more than 75 consecutive amino acids of SEQ ID NO: 13, such as no more than 70, 65, 60, 55, 45, 40, 35, 30, 25, 20, 15 or 14 consecutive amino acids of SEQ ID NO: 13. In more embodiments, the polypeptide does not include any additional consecutive amino acids of SEQ ID NO: 13 in addition to the sequence of the Hbb peptide.

The Hbb peptide sequences disclosed herein are listed in N to C terminal order. However, in some embodiments, the Hbb peptide sequence, shown above, is in C to N terminal order. Any of the disclosed Hbb peptides, in C to N terminal order, can be used in the methods disclosed herein.

In some embodiments, the isolated polypeptide includes a cell penetrating peptide linked to the Hbb peptide. In some embodiments, the cell penetrating peptide is YGRKKRRQRRR (SEQ ID NO: 14). Additional cell penetrating peptides are listed below:

```
  1) Penetratin
(RQIKIWFQNRRMKWKKGG, SEQ ID NO: 15)
  2) HIV-TAT (Variation)
(GRKKRRQRRRPQ, SEQ ID NO: 16)
  3) HIV-1 Rev-(34-50)
(SEQ ID NO: 53)
(TRQARRNRRRRWRERQR)
  4) PTD-4
(YARAAARQARA, SEQ ID NO: 17)
  5) Transportan
(GWTLNSAGYLLGKINLKALAALAKKIL, SEQ ID NO: 18)
  6) Ig(v)
(MGLGLHLLVLAAALQGAKKKRKV, SEQ ID NO: 19)
  7) kalata B1 (cysteine knot cyclic peptide)
(CGETCVGGTCNTPGCTCSWPVCTRNGLPV, SEQ ID NO: 20)
  8) MAP:
(KLALKLALKALKAALKLA, SEQ ID NO: 21)
  9) MPG:
(GALFLGFLGAAGSTMGAWSQPKKKRKV, SEQ ID NO: 22)
10) Pep-1
(KETWWETWWTEWSQPKKKRKV, SEQ ID NO: 23)
11) Pep-7
(SDLWEMMMVSLACQY, SEQ ID NO: 24)
12) Pept 1
(PLILLRLLRGQF, SEQ ID NO: 25)
13) Pept 2
(PLIYLRLLRGQF, SEQ ID NO: 26)
14) FGF
(PIEVCMYREP, SEQ ID NO: 27)
15) IVV-14
(KLWMRWYSPTTRRYG, SEQ ID NO: 28)
16) Amphiphilic model peptide
(KLALKLALKALKAALKLA, SEQ ID NO: 29)
17) pVEC
(LLIILRRRIRKQAHAHSK, SEQ ID NO: 30)
18) HRSV
(RRIPNRRPRR, SEQ ID NO: 31)
19) R6W3
(RRWWRRWRR, SEQ ID NO: 32)
20) Antp(43-58)
(RQIKIYFQNRRMKWKK, SEQ ID NO: 33)
21) FHV coat(35-49)
(RRRRNRTRRNRRRVR, SEQ ID NO: 34)
22) TP10
(AGYLLGKINLKALAALAKKIL, SEQ ID NO: 35)
23) EB1
(LIRLWSHLIHIWFQNRRLKWKKK, SEQ ID NO: 36)
24) M918
(MVTVLFRRLRIRRACGPPRVRV, SEQ ID NO: 37)
25) YTA2
(YTAIAWVKAFIRKLRK, SEQ ID NO: 38)
26) YTA4
(IAWVKAFIRKLRKGPLG, SEQ ID NO: 39)
27) NLS
(CGYGPKKKRKVGG, SEQ ID NO: 40)
28) ARF(1-22)
(MVRRFLVTLRIRRACGPPRVRV, SEQ ID NO: 41)
29) BPrPp(1-28)
(MVKSKIGSWILVLFVSDVGLCKKRP, SEQ ID NO: 42)
30) VP22
(NAATATRGRSAASRPTQRPRAPARSASRPRRPVQ, SEQ ID NO: 43)
31) MTS peptide
(KGEGAAVLLPVLLAAPG, SEQ ID NO: 44)
32) VT5
(DPKGDPKGVTVTVTVTVTGKGDPKPD, SEQ ID NO: 45)
33) Poly-arginine molecules
(e.g. R12-RRRRRRRRRRRRGC, SEQ ID NO: 46)
34) Poly-lysine molecules
(e.g. 8-Lysine-KKKKKKKK, SEQ ID NO: 47)
```

Additional cell penetrating peptides are disclosed in Derakhshankhah, H. & Jafari, S. Biomedicine & Pharmacotherapy 108, 1090-1096 (2018); Chauhan et al. J. Controlled Release 117, 148-162 (2007).; Bechara, C. & Sagan, S. FEBS Letters 587, 1693-1702 (2013); Patel et al. Pharmaceutical Research 24, 1977-1992 (2007); Borrelli, A et al. Molecules 23, 295 (2018); Guo, Z. et al. Biomedical Reports 4, 528-534 (2016).

See also Traboulsi et al., Bioconjugate Chemistry 26: 405-411, 2015; Splith and Neundorf, Eur. Biophys J 40: 387-397, 2011; and Foged and Nielsen, Expert Opinion on Drug Delivery, 5:1, 105-117, DOI:10.1517/17425247.5.1.105, 2007). The isolated polypeptide can include any of these cell penetrating peptides. The cell penetrating peptide can be amino terminal to the Hbb peptide. The cell penetrating peptide can be carboxy terminal to the Hbb peptide.

In some embodiments, the polypeptide includes a heterologous peptide linker between the cell penetrating peptide and the Hbb peptide. The heterologous peptide linker can be composed of glycine and/or serine residues. In some embodiments, the heterologous peptide linker is 4 or 5 amino acids in length. In more embodiments, the heterologous peptide linker is 2 to 15 amino acids in length, such as 2, 3, 4, 5, 6, 7, 8, 9, 10, 11, 12, 13, 14 or 15 amino acids, for example glycine and/or serine residues. Exemplary heterologous peptide linkers include, but are not limited to, GGGGS (SEQ ID NO: 48), GGGGSGGGGS (SEQ ID NO: 49) or GGGGSGGGGSGGGGS (SEQ ID NO: 50). In some embodiments, the disclosed polypeptide is of the format A-B-C, from amino to carboxy terminal end, wherein A is the Hbb peptide, B is an optional heterologous peptide linker, and C is the cell penetrating peptide. In other embodiments, the polypeptide is of the format C-B-A, from amino to carboxy terminal end, wherein C is the cell penetrating peptide, B is an optional heterologous peptide linker, and A is the Hbb peptide.

In further embodiments, the polypeptide includes only one Hbb peptide. In a specific non-limiting example, the cell penetrating peptide includes, or consists of SEQ ID NO: 14. The isolated polypeptide can include, or consist of, SEQ ID NO: 14 and one of SEQ ID NOs: 4, 5 and 6. The isolated polypeptide can include, or consist of SEQ ID NO: 14 and SEQ ID NO: 4. The isolated polypeptide can include, or consist of SEQ ID NO: 14 and SEQ ID NO: 5. The isolated polypeptide can include, or consist of SEQ ID NO: 14 and SEQ ID NO: 6. The cell penetrating peptide of SEQ ID NO: 14 can be amino terminal to the cell penetrating peptide, or carboxy terminal to the cell penetrating peptide. Optionally, a heterologous linker, such as a heterologous peptide linker, is included between the cell penetrating peptide and the Hbb peptide (SEQ ID NO: 4, 5, or 6).

In some non-limiting examples, a polypeptide of use in the disclosed methods comprises SEQ ID NO: 14 and SEQ ID NO: 4. The cell penetrating peptide (SEQ ID NO: 14) can be N terminal to the Hbb peptide. Alternatively, the cell penetrating peptide (SEQ ID NO: 14) can be C terminal to the Hbb peptide. In these non-limiting examples, a heterologous peptide linker can be included between the cell penetrating peptide and the Hbb peptide. The heterologous peptide linker can be GGGGS (SEQ ID NO: 48). The heterologous peptide linker can be GGGGSGGGGS (SEQ ID NO: 49). The heterologous peptide linker can be GGGGSGGGGSGGGGS (SEQ ID NO: 50). The polypeptide can consist essentially of, or consist of, the cell penetrating peptide, the heterologous peptide linker, and the Hbb peptide. The polypeptide can consist essentially of, or consist of, the cell penetrating peptide and the Hbb peptide.

In other non-limiting examples, a polypeptide of use in the disclosed methods comprises SEQ ID NO: 14 and SEQ ID NO: 5. The cell penetrating peptide (SEQ ID NO: 14) can be N terminal to the Hbb peptide. Alternatively, the cell penetrating peptide (SEQ ID NO: 14) can be C terminal to the Hbb peptide. In these non-limiting examples, a heterologous peptide linker can be included between the cell penetrating peptide and the Hbb peptide. The heterologous peptide linker can be GGGGS (SEQ ID NO: 48). The heterologous peptide linker can be GGGGSGGGGS (SEQ ID NO: 49). The heterologous peptide linker can be GGGGSGGGGSGGGGS (SEQ ID NO: 50). The polypeptide can consist essentially of, or consist of, the cell penetrating peptide, the heterologous peptide linker, and the Hbb peptide. The polypeptide can consist essentially of, or consist of, the cell penetrating peptide and the Hbb peptide.

In some non-limiting examples, a polypeptide of use in the disclosed methods comprises SEQ ID NO: 14 and SEQ ID NO: 6. The cell penetrating peptide (SEQ ID NO: 14) can be N terminal to the Hbb peptide. Alternatively, the cell penetrating peptide (SEQ ID NO: 14) can be C terminal to the Hbb peptide. In these non-limiting examples, a heterologous peptide linker can be included between the cell penetrating peptide and the Hbb peptide. The heterologous peptide linker can be GGGGS (SEQ ID NO: 48). The heterologous peptide linker can be GGGGSGGGGS (SEQ ID NO: 49). The heterologous peptide linker can be GGGGSGGGGSGGGGS (SEQ ID NO: 50). The polypeptide can consist essentially of, or consist of, the cell penetrating peptide, the heterologous peptide linker, and the Hbb peptide. The polypeptide can consist essentially of, or consist of, the cell penetrating peptide and the Hbb peptide.

The disclosed polypeptides can be at most 75 amino acids in length. The disclosed polypeptide can be, for example, 10-14 amino acids in length, such as 10, 11, 12, 13 or 14 amino acids in length. The polypeptide can be, for example, 10-75, 10-50, or 10-25 amino acids in length. The polypeptide can be, for example, 11-75, 11-50, or 11-25 amino acids in length. The polypeptide can be, for example, 14-75, 14-50, or 14-25 amino acids in length

The polypeptide can be, for example, 10-14, 10-20, 10-30, 10-40, 10-50, 10-60, or 10-70 amino acids in length. The polypeptide can be for example, 11-14, 11-20, 11-30, 11-40, 11-50, 11-60, or 11-70 amino acids in length. The polypeptide can be, for example, 14-20, 14-30, 14-40, 14-50, 14-60, or 14-70 amino acids in length. The polypeptide can be, for example, 10, 15, 20, 25, 30, 35, 40, 45, 50, 55, 60, 65, 70 or 75 amino acids in length.

In some embodiments, the peptide is pegylated, polymerized or cross-linked.

In some embodiments, the disclosed peptide is conjugated to a heterologous moiety. For example, Conjugates can be produced that include a disclosed polypeptide and an HbαX peptide. HbαX peptides are disclosed for example, in U.S. Pat. Nos. 10,314,883, 10,253,069 and 9,701,714. The Hbα peptide can include, or consist of, LSFPTTKTYF (SEQ ID NO: 51), or LSFPTTKTYFPHFDLSHGSA (SEQ ID NO: 52). In some embodiments, the HbαX peptide is no more than 20 amino acids in length. The HbαX peptide can be no more that 10, 11, 12, 13, 14, 15, 16, 17, 18, or 19 amino acids in length. The HbαX can be, for example, 10-15 or 15-20 amino acids in length. The HbαX peptide can be 10 or 20 amino acids in length.

In some embodiments, a heterologous linker is included between the polypeptide and the HbαX peptide. In some non-limiting examples, the heterologous linker is 16 Angstroms. Suitable heterologous linkers include, but are not limited to, linkers is —NH—(CH₂)ₙCO— where n=4-20. Additional heterologous linkers include, but are not limited to, —NH—CH₂—CH₂—(O— CH₂CH₂)ₙOCH₂CO— where n=1-12. In more non-limiting examples, the heterologous linker can be linker is NHCH₂-Aryl-O-Aryl-CO₂H, where Aryl is 1-4 substituted benzene, or 2,5-substituted pyridine, or 2,5-disubstituted pyrazine, or a mixture thereof. Specific examples include, but are not limited to: 4-(4-(aminomethyl)phenoxy)benzoic acid, 4-(4-aminophenoxy)benzoic acid, 4-(4-(aminomethyl)benzyl)benzoic acid, 4-(4-aminobenzyl)benzoic acid, 4′-(aminomethyl)-[1,1′-biphenyl]-4-carboxylic acid, 4′-amino-[1,1′-biphenyl]-4-carboxylic acid, 6-(4-(aminomethyl)phenoxy)nicotinic acid, 6-(4-aminophenoxy)nicotinic acid, 6-((6-(aminomethyl)pyridin-3-yl)oxy)nicotinic acid, 6-((6-aminopyridin-3-yl)oxy)nicotinic acid, 5-(4-(aminomethyl)phenoxy)pyrazine-2-carboxylic acid, and 5-(4-aminophenoxy)pyrazine-2-carboxylic acid.

In further embodiments, the heterologous linker is two 8-amino-3,6-dioxaoctanoic acid moieties. Non-limiting examples of the disclosed conjugates are shown below:

a.
YGRKKRRQRRR (SEQ ID NO: 14)-LSFPTTKTYF (SEQ ID NO: 51)-
[heterologous linker]-TPEEKSAVTALWGK (SEQ ID NO: 4);
b.
LSFPTTKTYF (SEQ ID NO: 51)-[heterologous linker]-
TPEEKSAVTALWGK (SEQ ID NO: 4)-YGRKKRRQRRR (SEQ ID NO: 14);
c.
YGRKKRRQRRR (SEQ ID NO: 14)-LSFPTTKTYFPHFDLSHGSA (SEQ
ID NO: 52)-|heterologous linker|-TPEEKSAVTALWGK (SEQ ID NO: 4);
and
d.
LSFPTTKTYFPHFDLSHGSA (SEQ ID NO: 52)-[heterologous linker]-
TPEEKSAVTALWGK (SEQ ID NO: 4)-YGRKKRRQRRR (SEQ ID NO: 14),

wherein X is 8-amino-3,6-dioxaoctanoic acid. See, for example, Biopolymers 2006, 84, 576-585. Additional non-limiting examples of the disclosed conjugates are shown below:

a.
YGRKKRRQRRR (SEQ ID NO: 14)-LSFPTTKTYF (SEQ ID NO: 51)-
[heterologous linker]-HFGKEFTPPV (SEQ ID NO: 5);
b.
LSFPTTKTYF (SEQ ID NO: 51)-[heterologous linker]-HFGKEFTPPV
(SEQ ID NO: 5)-YGRKKRRQRRR (SEQ ID NO: 14);
c.
YGRKKRRQRRR (SEQ ID NO: 14)-LSFPTTKTYFPHFDLSHGSA (SEQ
ID NO: 52)-[heterologous linker]-HFGKEFTPPV (SEQ ID NO: 5);
and
d.
LSFPTTKTYFPHFDLSHGSA (SEQ ID NO: 52)-[heterologous linker]-
HFGKEFTPPV (SEQ ID NO: 5)-YGRKKRRQRRR (SEQ ID NO: 14),

wherein X is 8-amino-3,6-dioxaoctanoic acid.

More non-limiting examples of the disclosed conjugate are shown below:

a.
YGRKKRRQRRR (SEQ ID NO: 14)-LSFPTTKTYF (SEQ ID NO: 51)-
[heterologous linker]-TLSELHCDKLH (SEQ ID NO: 6);
b.
LSFPTTKTYF (SEQ ID NO: 51-[heterologous linker]-TLSELHCDKLH
(SEQ ID NO: 6)-YGRKKRRQRRR (SEQ ID NO: 14);
c.
YGRKKRRQRRR (SEQ ID NO: 14)-LSFPTTKTYFPHFDLSHGSA (SEQ
ID NO: 52)-[heterologous linker]-TLSELHCDKLH (SEQ ID NO: 6);
and
d.
LSFPTTKTYFPHFDLSHGSA (SEQ ID NO: 52)-[heterologous linker]-
TLSELHCDKLH (SEQ ID NO: 6)-YGRKKRRQRRR (SEQ ID NO: 14),

wherein X is 8-amino-3,6-dioxaoctanoic acid.

In other embodiments, isolated agents are also provided that include the disclosed polypeptide and a second Hbb peptide, wherein the second Hbb peptide consists of:
a. X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid;
b. HFX₇KEX₈X₉PPV (SEQ ID NO: 2), wherein X₇ is any amino acid, X₈ is any amino acid, and X₉ is T or S; or
c. TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar uncharged amino acid, X₁₄ is C, M or T, and X₁5 is any amino acid.

Any of the Hbb peptides disclosed above are of use.

These agents can be fusion proteins. In some embodiments, the agent includes comprising a heterologous linker between the isolated polypeptide and the second Hbb peptide. In specific non-limiting examples, the heterologous linker is a heterologous peptide linker, as discussed above.

The heterologous peptide linker can be, for example, GGGGS (SEQ ID NO: 48), GGGGSGGGGS (SEQ ID NO: 49) or GGGGSGGGGSGGGGS (SEQ ID NO: 50).

Exemplary fusion proteins include, but are not limited to, fusion proteins that include SEQ ID NO: 4 and SEQ ID NO: 5, SEQ ID NO: 5 and SEQ ID NO: 6, or SEQ ID NO: 4 and SEQ ID NO: 6. Optionally, a heterologous peptide linker can be included between the two Hbb peptides, such as, but not limited to GGGGS (SEQ ID NO: 48), GGGGSGGGGS (SEQ ID NO: 49) or GGGGSGGGGSGGGGS (SEQ ID NO: 50). In more embodiments, the fusion protein includes A-B-C-D-E, in amino to carboxy terminal order, wherein A is a cell penetrating peptide, B is an optional heterologous peptide linker, C is a first Hbb peptide, D is an optional heterologous peptide linker, and E is a second Hbb peptide. In more embodiments, the fusion protein includes F-G-H-I-J, in amino to carboxy terminal order, wherein F is a first Hbb peptide, G is an optional heterologous peptide linker, H is a second Hbb peptide, I is an optional heterologous peptide linker and G is a cell penetrating peptide. In further embodiments HbαX peptide can be present.

Isolated agents are also provided that include the disclosed conjugate, with a chemical linker, and a second Hbb peptide, wherein the second Hbb peptide consists of:
a. X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid;
b. HFX₇KEX₈X₉PPV (SEQ ID NO: 2), wherein X₇ is any amino acid, X₈ is any amino acid, and X₉ is T or S; or
c. TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁₅ is any amino acid.

Any of the Hbb peptides disclosed above are of use in these agents. Exemplary Hbb peptides are SEQ ID NO: 4, SEQ ID NO: 5, or SEQ ID NO: 6. In some embodiments, an agent of use in the disclosed methods includes two different Hbb peptides. In other embodiments, of conjugate of use in the disclosed methods includes all three of these Hbb peptides. In yet other embodiments, a conjugate of use in the disclosed methods includes 2, 3, 4, or 5 copies of the same Hbb peptide.

In some embodiments, the agent includes a heterologous linker between the isolated polypeptide and the second Hbb peptide. In specific non-limiting examples, the linker is a peptide linker, as discussed above. Optionally, an HbαX peptide can also be included, as disclosed above. The peptides can be prepared by solution or solid phase peptide synthesis using standard fluorenylmethoxyoxycarbonyl (Fmoc) or tert-butoxycarbonyl (Boc) amino acid protecting groups and coupling agents such as diisopropyl carbodiimide (DIC), dicyclohexylcarbodiimide (DCC), or HATU etc. The peptides can also be prepared using recombination methods. The peptides also be administered in a sustained release depot formulation using polylactide degrading polymers or other biological polymers commonly used for this purpose. Solid or solution phase peptide synthesis can be used to introduce unnatural amino acids into the peptide sequence. Examples include L-2-naphthyl alanine, L-1-naphthyl alanine, L-Biphenylalanine. The peptide sequence can also be modified at the N-terminus by coupling with carboxylic acids such as acetic or benzoic acid. In some embodiments, the C-terminus of the peptide can be amidated.

### II. Polynucleotides and Expression

Polynucleotides encoding a protomer of any of the disclosed polypeptides, Hbb peptides, and fusion proteins are also provided. These polynucleotides include DNA, cDNA and RNA sequences which encode the peptide, as well as vectors including the DNA, cDNA and RNA sequences, such as a DNA or RNA vector. The genetic code to construct a variety of functionally equivalent nucleic acids, such as nucleic acids which differ in sequence but which encode the same protein sequence, or encode a conjugate or fusion protein.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a disclosed polypeptide, Hbb peptides and fusion proteins thereof can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a disclosed polypeptide, Hbb peptide or fusion protein can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. The promoter can be either inducible or constitutive. An inducible promoter is a promoter that is inactive or exhibits low activity except in the presence of an inducer substance. Additional examples of promoters include, but are not limited to, MT II, MMTV, collagenase, stromelysin, SV40, murine MX gene, α-2-macroglobulin, MHC class I gene h-2kb, HSP70, proliferin, tetracycline inducible, tumor necrosis factor, or thyroid stimulating hormone gene promoter. One example of an inducible promoter is the interferon inducible ISG54 promoter (see Bluyssen et al., Proc. Natl Acad. Sci. 92: 5645-5649, 1995, herein incorporated by reference).

In some embodiments, the promoter is a constitutive promoter that results in high levels of transcription upon introduction into a host cell in the absence of additional factors. Other promoters include promoters specific to endothelial cells.

Exemplary procedures sufficient to guide one of ordinary skill in the art through the production of a vector capable of expression in a host cell that includes a promoter, and/or a polynucleotide sequence encoding disclosed peptide can be found, for example, in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

It may be desirable to include a polyadenylation signal to effect proper termination and polyadenylation of the gene transcript. Exemplary polyadenylation signals have been isolated from beta globin, bovine growth hormone, SV40, and the herpes simplex virus thymidine kinase genes.

DNA sequences encoding a disclosed polypeptide, Hbb peptide or fusion protein can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and W138, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques. Where the host is prokaryotic, such as, but not limited to, E. coli, competent cells which are capable of DNA uptake can be prepared from cells harvested after exponential growth phase and subsequently treated by the CaCl₂ method using standard procedures. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

Modifications can be made to a nucleic acid encoding a disclosed polypeptide, Hbb peptide or fusion protein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

### III. Viral Vectors

A nucleic acid molecule encoding a disclosed polypeptide, Hbb peptide or fusion protein can be included in a viral vector, for example, for expression in a host cell, or for treatment of a subject as disclosed herein.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express proteins and peptides, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, lentivirus vectors and poliovirus vectors. Specific exemplary vectors are poxvirus vectors, such as vaccinia virus, fowlpox virus and a highly attenuated vaccinia virus (MVA), adenovirus, baculovirus, yeast, and the like. Adeno-associated virus vectors (AAV) are disclosed in additional detail below, and are of use in the disclosed methods. Defective viruses, that entirely or almost entirely lack viral genes, can be used. The vector can be a lentiviral vector. Use of defective viral vectors allows for administration to specific cells without concern that the vector can infect other cells.

In several embodiments, the viral vector can include an adenoviral vector that expresses the polypeptide, Hbb peptide or fusion protein. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/022311.

Further provided are recombinant vectors, such as recombinant adenovirus vectors and recombinant adeno-associated virus (rAAV) vectors comprising a nucleic acid molecule(s) disclosed herein. In some embodiments, the AAV is rAAV8, and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some embodiments, these elements are included in the AAV vector.

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some embodiments, these elements are included in the AAV vector.

AAV vectors can be used for gene therapy. Exemplary AAV of use are AAV2, AAV5, AAV6, AAV8 and AAV9. In some embodiments, a rAAV2 or rAAV8 vector can be used in the methods disclosed herein. However, rAAV6 and rAAV9 vectors are also of use.

Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of an rAAV for the methods disclosed herein.

AAV possesses several additional desirable features for therapy, including the ability to bind and enter target cells, enter the nucleus, the ability to be expressed in the nucleus for a prolonged period of time, and low toxicity. AAV can be used to transfect cells, and suitable vector are known in the art, see for example, U.S. Published Patent Application No. 2014/0037585, incorporated herein by reference. Methods for producing rAAV suitable for gene therapy are well known in the art (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

In some embodiments, the vector is a rAAV8 vector, a rAAV2 vector, a rAAV9 vector. In a specific non-limiting example, the vector is an AAV8 vector. AAV8 vectors are disclosed, for example, in U.S. Pat. No. 8,692,332, which is incorporated by reference herein. The location and sequence of the capsid, rep 68/78, rep 40/52, VP1, VP2 and VP3 are disclosed in this U.S. Pat. No. 8,692,332. The location and hypervariable regions of AAV8 are also provided. In some embodiments, the vector is an AAV2 variant vector, such as AAV7m8.

The vectors of use in the methods disclosed herein can contain nucleic acid sequences encoding an intact AAV capsid which may be from a single AAV serotype (e.g., AAV2, AAV6, AAV8 or AAV9). As disclosed in U.S. Pat. No. 8,692,332, vectors of use can also be recombinant, and thus can contain sequences encoding artificial capsids which contain one or more fragments of the AAV8 capsid fused to heterologous AAV or non-AAV capsid proteins (or fragments thereof). These artificial capsid proteins are selected from non-contiguous portions of the AAV2, AAV6, AAV8 or AAV9 capsid or from capsids of other AAV serotypes. For example, a rAAV vector may have a capsid protein comprising one or more of the AAV8 capsid regions selected from the VP2 and/or VP3, or from VP1, or fragments thereof selected from amino acids 1 to 184, amino acids 199 to 259; amino acids 274 to 446; amino acids 603 to 659; amino acids 670 to 706; amino acids 724 to 738 of the AAV8 capsid, which is presented as SEQ ID NO: 2 in U.S. Pat. No. 8,692,332. In another example, it may be desirable to alter the start codon of the VP3 protein to GTG. Alternatively, the rAAV may contain one or more of the AAV serotype 8 capsid protein hypervariable regions, for example aa 185-198; aa 260-273; aa447-477; aa495-602; aa660-669; and aa707-723 of the AAV8 capsid which is presented as SEQ ID NO: 2 in U.S. Pat. No. 8,692,332.

### IV. mRNA

The presently disclosed methods can utilize mRNA encoding a disclosed polypeptide, Hbb peptide, or fusion protein. In one embodiment, an mRNA of use includes an in vitro-transcribed nucleic acid. The RNA is produced by in vitro transcription using a plasmid DNA template generated synthetically. DNA of interest from any source can be directly converted by PCR into a template for in vitro mRNA synthesis using appropriate primers and RNA polymerase. In various embodiments, plasmid is used to generate a template for in vitro transcription of mRNA which is used in the disclosed methods.

In some embodiments, the mRNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach the 5′ and 3′ UTR lengths can be modified as needed to increase translation efficiency following transfection of the transcribed RNA

The 5′ and 3′ UTRs can be the naturally occurring, endogenous 5′ and 3′ UTRs for the gene encoding a globin. Alternatively, UTR sequences that are not endogenous to the gene of interest can be added by incorporating the UTR sequences into the forward and reverse primers or by any other modifications of the template. Without being bound by theory, the use of UTR sequences that are not endogenous to the gene of interest can be useful for modifying the stability and/or translation efficiency of the RNA. Without being bound by theory, ALU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain a Kozak sequence Kozak sequences can increase the efficiency of translation of some RNA transcripts, but are not required for all RNAs to enable efficient translation.

Thus, in some embodiments, the mRNAs that encode the disclosed polypeptide, Hbb peptide or fusion protein include a 5′ UTR and/or a 3′ UTR that results in greater mRNA stability and higher expression of the mRNA in the cells.

In some embodiments, the mRNA includes a Kozak sequence in the 5′ UTR. The Kozak sequence can be, for example, ACCAUGG.

In some embodiments, the mRNA is polyadenylated. In some embodiments, the mRNA comprises a poly-A tail (e.g., a poly-A tail having 50-200 nucleotides, such as 100-200, 150-200 nucleotides, or greater than 100 nucleotides), although in some embodiments, a longer or a shorter poly-A tail is used. In some embodiments, the poly A tail is 100, 101, 102, 103, 104, 105, 106, 107, 108, 109, or 110 nucleotides in length.

The recombinant mRNA can include a 5′ capping structure. 5′-capping of modified RNA can be completed concomitantly during IVT using the following chemical RNA cap analogs to generate the 5′-guanosine cap structure: 3′-O-Me-m7G(5′)ppp(5′)G; G(5′)ppp(5′)A; G(5′)ppp(5′)G; m7G(5′)ppp(5′)A; m7G(5′)ppp(5′)G (New England BioLabs, Ipswich, Mass.). In some embodiments, 5′-capping of modified RNA may be completed post-transcriptionally using a Vaccinia Vims Capping Enzyme to generate the “Cap 0” structure: n7G(5′)ppp(5′)G (New England BioLabs, Ipswich, Mass.). Cap 1 structure can be generated using both Vaccinia ViJ.us Capping Enzyme and a 2′-0 methyl-transferase to generate: m7G(5′)ppp(5′)G-2′-O-methyl. Cap 2 structure can be generated from the Cap 1 structure followed by the 2′-O-methylation of the 5′-antepenultimate nucleotide using a 2′-0 methyl-transferase. Cap 3 structure can be generated from the Cap 2 structure followed by the 2′-O-methylation of the 5′-preantepenultimate nucleotide using a 2′-0 methyl-transferase. See U.S. Pat. No. 9,701,965.

To enable synthesis of RNA from a DNA template without the need for gene cloning, a promoter of transcription can be attached to the DNA template, upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described in U.S. Published Patent Application No. 2016/0030527A1. Other useful promoters include, but are not limited to, T3 and SP6 RNA polymerase promoters. Consensus nucleotide sequences for T7, T3 and SP6 promoters are known in the art.

The mRNA can be prepared using in vitro transcription (IVT). The IVT can be performed using any RNA polymerase as long as synthesis of the mRNA from the DNA template that encodes the RNA is specifically and sufficiently initiated from a respective cognate RNA polymerase promoter and full-length mRNA is obtained. In some preferred embodiments, the RNA polymerase is selected from among T7 RNA polymerase, SP6 RNA polymerase and T3 RNA polymerase. In some other embodiments, capped RNA is synthesized co-transcriptionally by using a dinucleotide cap analog in the IVT reaction (e.g., using an AMPLICAP™ T7 Kit or a MESSAGEM AX™ T7 ARCA-CAPPED MESSAGE Transcription Kit: EPICENTRE or CellScript, Madison. Wis., USA). If capping is performed co-transcriptionally, the dinucleotide cap analog can be an anti-reverse cap analog (ARCA). However, use of a separate IVT reaction, followed by capping with a capping enzyme system, which results in approximately 100% of the RNA being capped. Another option is co-transcriptional capping, which typically results in only about 80% of the RNA being capped. Thus, in some embodiments, a high percentage of the mRNA molecules are capped (e.g., greater than 80%, greater than 90%, greater than 95%, greater than 98′, greater than 99%, greater than 99.5%, or greater than 99.9% of the population of mRNA molecules are capped).

In more embodiments, the mRNA can be prepared by polyadenylation of an in vitro-transcribed (iVT) RNA using a poly(A) polymerase (e.g., yeast RNA polymerase or E. coli poly(A) polymerase). In some embodiments, the mRNA is polyadenylated during in vitro transcription (IVT) by using a DNA template that encodes the poly(A) tail.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase.

The mRNA sequence can include, in 5′ to 3′ order, a Cap, a 5′UTR including a Kozak sequence, a codon optimized sequence encoding a disclosed polypeptide, Hbb peptide or fusion protein, and a poly A tail.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985): Nacheva and Berzal-Herranz, Eur. J. Biochem., 270:1485-65 (2003). The conventional method of integration of polyA, f stretches into a DNA template is molecular cloning. If a polyA/T sequence integrated into plasmid DNA can cause plasmid instability in some cells, then this instability can be ameliorated through the use of recombination incompetent bacterial cells for plasmid propagation.

This disclosure includes the use of RNAs that contain one or more modified nucleosides (termed “modified nucleic acids”), which have useful properties including the lack of a substantial induction of the innate immune response of a cell into which the mRNA is introduced. As disclosed in U.S. Pat. No. 9,701,965, these modified nucleic acids enhance the efficiency of protein production, intracellular retention of nucleic acids, and viability of contacted cells, as well as possess reduced immunogenicity.

Provided are modified nucleic acids, such as a recombinant mRNA that includes one, two, or more than two different nucleoside modifications. In some embodiments, the modified nucleic acid exhibits reduced degradation in a cell into which the nucleic acid is introduced, relative to a corresponding unmodified nucleic acid. For example, the degradation rate of the modified nucleic acid is reduced by about 10%, 20%, 30%, 40%, 50%, 60%, 70%, 80%, 90%, or greater than 90%, compared to the degradation rate of the corresponding unmodified nucleic acid. These nucleic acids do not substantially induce an innate immune response of a cell into which the mRNA is introduced.

In some embodiments, modified nucleosides include pyridin-4-one ribonucleoside, 5-aza-uridine, 2-thio-5-aza-uridine, 2-thiomidine, 4-thio-pseudomidine, 2-thio-pseudowidine, 5-hydroxyuridine, 3-methylmidine, 5-carboxymethyl-uridine, 1-carboxymethyl-pseudoutidine, 5-propynyl-uridine, 1-propynyl-pseudomidine, 5-taurinomethyluridine, 1-taurinomethyl-pseudouridine, 5-taw.inomethyl-2-thio-utidine, 1-taurinomethyl-4-thio-uridine, 5-methyl-uridine, 1-methyl-pseudouridine, 4-thio-1-methyl-pseudouridine, 2-thio-1-methyl-pseudoutidine, 1-methyl-1-deaza-pseudomidine, 2-thio-1-methyl-1-deaza-pseudouridine, dihydrouridine, dihydropseudouridine, 2-thio-dihydromidine, 2-thio-dihydropseudoulidine, 2-methoxyuridine, 2-methoxy-4-thio-uridine, 4-methoxy-pseudomidine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebulruine, 5-methyl-zebularine, 5-aza-2-thio-zebulru.ine, 2-thio-zebulaiine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-dianinopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In specific embodiments, a modified nucleoside is 5′-O-(1-Thiophosphate)-Adenosine, 5′-O-(1-Thiophosphate)-Cytidine, 5′-O-(1-thiophosphate)-Guanosine, 5′-O-(1-Thiophophate)-Uridine or 5′-O-(1-Thiophosphate)-Pseudouridine.

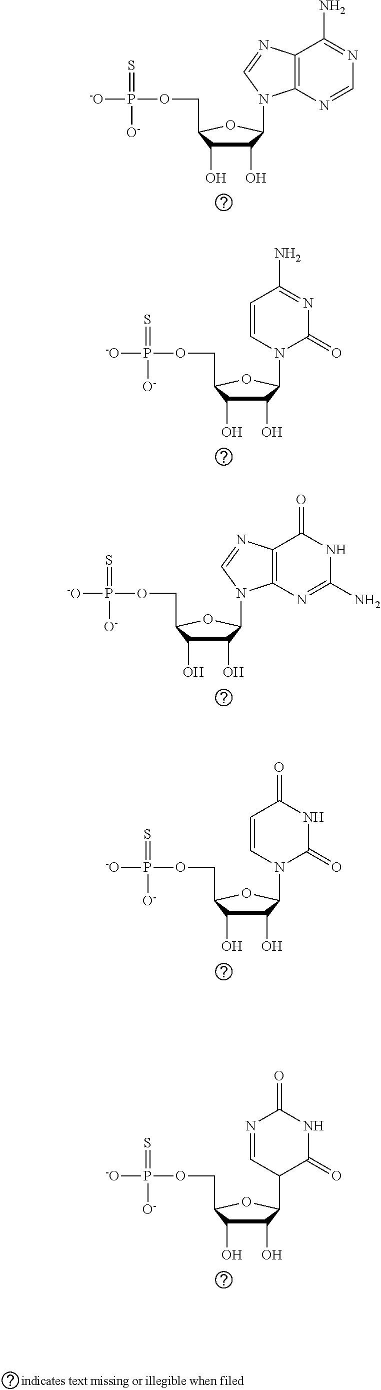

As disclosed in U.S. Pat. No. 9,701,965, the α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked nucleic acids are expected to also reduce the innate immune response through weaker binding/activation of cellular innate immune molecules.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, J-methyl-6-thio-guanosine, N2-methyl-6-thio-guanosine, and N2,N2-dimethyl-6-thio-guanosine.

The disclosed mRNA can include a modified uridine or 1-methylpseudouridine. mRNA that contain either uridine, or 1-methylpseudouridine in place of uridine, the 1-methylpseudouridine-containing mRNA was translated at a higher level or for a longer duration than the mRNA that contained uridine. Therefore, in some embodiments, one or more or all of the uridines contained in the mRNA(s) used in the methods disclosed herein is/are replaced by 1-methylpseudouridine (such as by substituting 1-methylpseudouridine-5′-triphosphate in an IVT reaction to synthesize the RNA in place of uridine-5′-triphosphate). However, in some embodiments, the mRNA used in the disclosed methods contains uridine and does not contain 1-methylpseudouridine. In more embodiments, the mRNA comprises at least one modified nucleoside (e.g., 1-methylpseudouridine (mIψ), pseudouridine (ψ), 5-methylcytosine (m⁵C), 5-methyluridine (m⁵U), 2′-O-nethyluridine (Um or m^{2′-OU}), 2-thiouridine (s²U), or N⁶-methyladenosine (m⁶A)) in place of at least a portion of the corresponding unmodified canonical nucleoside (e.g., in place of substantially all of the corresponding unmodified A, C, G, or T canonical nucleoside). In some embodiments, the mRNA comprises at least one modified nucleoside wherein the nucleotide is pseudouridine (ψ) or 5-nethyleytosine (m⁵C). In some embodiments, the mRNA comprises both pseudouridine (ψ) and 5-methyleytosine (m⁵C). In other embodiments, the mRNA includes 1-methylpseudouridine.

In addition, in some embodiments, a nucleic acid base, sugar moiety, or internucleotide linkage in one or more of the nucleotides of the mRNA that is introduced into a eukaryotic cell in any of the methods disclosed herein can comprise a modified nucleic acid base, sugar moiety, or internucleotide linkage.

The modified nucleic acids, as disclosed herein, are capable of evading an innate immune response of a cell into which the nucleic acids are introduced, thus increasing the efficiency of protein production in the cell. While it is advantageous to eliminate the innate immune response in a cell, the disclosure provides modified mRNAs that substantially reduce the immune response, including interferon signaling, without entirely eliminating such a response. In some embodiments, the immune response is reduced by about 10%, 20%, 30%, 40%, 50%, 60%, 70%, 80%, 90%, 95%, 99%, 99.9%, or greater than 99.9%, as compared to the immune response induced by a corresponding unmodified nucleic acid. Such a reduction can be measured by expression or activity level of type 1 interferons or the expression of interferon-regulated genes such as the toll-like receptors (e.g., TLR7 and TLR8). Reduction of innate immune response can also be measured by decreased cell death following one or more administrations of modified RNAs to a cell population; e.g., cell death is about 10%, 25%, 50%, 75%, 85%, 90%, 95%, or over 95% less than the cell death frequency observed with a corresponding unmodified nucleic acid. Moreover, cell death may affect fewer than about 50%, 40%, 30%, 20%, 10%, 5%, 1%, 0.1%, 0.01%, or fewer than 0.01% of cells contacted with the modified nucleic acids.

Nucleic acids encoding for use in accordance with the disclosure may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005).

Modified nucleic acids need not be uniformly modified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides.

When transfected into mammalian cells, the modified mRNA can have a stability of between 12-18 hours or more than 18 hours, such as about 24, 36, 48, 60, 72 or greater than about 72 hours. In some embodiments, the modified mRNA is stable for about 12 to about 72 hours, such as about 12 to about 48 hours, about 12 to about 36 hours, or about 12 to about 24 hours.

In a specific non-limiting example, the mRNA component is a modified mRNA with modified uridine, such as a 1-methylpseudouridine in place of uridine and a 7mG(5′)ppp(5′)N1mpNp cap.

Lipid Nanoparticles are disclosed, for example, in PCT Publication No. 2021/150891. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma. St. Louis. Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids. Inc. (Birmingham. Ala.). Stock solutions of lipids in chloroform or chloroform/methanol can be stored at about −20′ C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In an embodiment, an RNA molecule is encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171. 1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al., Electrolyte- and pH-stabilities of aqueous solid lipid nanoparticle (SLN) dispersions in artificial gastrointestinal media. Eur J Pharm Biopharm 52:203.2001). Methods are also disclosed in PCT Publication Nos. WO2021154763, US20210228707, WO2017070626 and US2019/0192646. See, also, Jackson et al., N Engl J Med., 383(20):1920-1921, 2020.

In several embodiments, the mRNA is formulated in a lipid nanoparticle for administration to the subject; for example, comprising a PEG-modified lipid, a non-cationic lipid, a sterol, an ionizable lipid, or any combination thereof. In some embodiments, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3,dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

### V. Pharmaceutical Compositions and Methods of Treatment

An effective amount of one or more of the disclosed polypeptide, Hbb peptide, fusion protein, conjugate, agent or nucleic acid molecule (including RNA, DNA and a vector), can be included in a pharmaceutical composition. Accordingly, provided herein are pharmaceutical compositions that include a polypeptide, Hbb peptide, fusion protein, conjugate, agent or nucleic acid molecule, as disclosed herein, such as an RNA or a vector, and one or more pharmaceutically acceptable excipients. These pharmaceutical compositions are of use in the disclosed methods.

The compositions can include one or more other active (therapeutic) ingredients in a pharmaceutically acceptable carrier. The carrier(s) are “acceptable” in the sense of being compatible with the other ingredients of the formulation and not deleterious to the recipient thereof. Proper formulation of the pharmaceutical composition is dependent upon several factors, such as the route of administration chosen. Any of the well-known techniques and excipients may be used as suitable and as understood in the art. The pharmaceutical compositions disclosed herein can be manufactured in any manner known in the art, e.g., by means of conventional mixing, dissolving, granulating, dragee-making, levigating, emulsifying, encapsulating, entrapping or compression processes.

In some embodiments, the composition includes one or more of the following excipients: N-acetyl cysteine, sodium citrate, glycine, histidine, glutamic acid, sorbitol, maltose, mannitol, trehalose, lactose, glucose, raffinose, dextrose, dextran, ficoll, gelatin, hydroxyethyl starch, benzalkonium chloride, benzethonium chloride, benzyl alcohol, chlorobutanol, m-cresol, myristyl gamma-picolinium chloride, paraben methyl, paraben propyl, 2-penoxythanol, phenyl mercuric nitrate, thimerosal, acetone sodium bisulfite, argon, ascorbyl palmitate, ascorbate (sodium/acid), bisulfite sodium, butylated hydroxy anisole (BHA), butylated hydroxy toluene (BHT), cysteine/cysteinate HCl, dithionite sodium (Na hydrosulfite, Na sulfoxylate), gentisic acid, gentisic acid ethanolamine, glutamate monosodium, glutathione, formaldehyde sulfoxylate sodium, metabisulfite potassium, metabisulfite sodium, methionine, monothioglycerol (thioglycerol), nitrogen, propyl gallate, sulfite sodium, tocopherol alpha, alpha tocopherol hydrogen succinate, and thioglycolate sodium. The present disclosure also contemplates other excipients, including any disclosed in Pramanick et al., Pharma Times 45(3): 65-77, 2013. The pharmaceutical compositions disclosed herein can be administered by a variety of routes, depending upon whether local or systemic treatment is desired and upon the area to be treated.

The pharmaceutical compositions include those suitable for parenteral (including subcutaneous, intradermal, intramuscular, intravenous, intraarticular, and intramedullary), or intraperitoneal administration, although the most suitable route may depend upon for example the condition and disorder of the recipient. Parenteral administration includes intravenous, intraarterial, subcutaneous, intraperitoneal, intramuscular or injection or infusion; or intracranial, e.g., intrathecal or intraventricular, administration. Parenteral administration can be in the form of a single bolus dose, or may be, for example, by a continuous perfusion pump. Conventional pharmaceutical carriers, aqueous, powder or oily bases, thickeners and the like may be necessary or desirable. In some embodiments, the compounds can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance. For example, Modern Pharmaceutics, 5th Edition, Banker & Rhodes, CRC Press (2009); and Goodman & Gilman's The Pharmaceutical Basis of Therapeutics, 13th Edition, McGraw Hill, New York (2018) can be consulted. The compositions can conveniently be presented in unit dosage form and may be prepared by any of the methods well known in the art of pharmacy. Typically, these methods include the step of bringing into association an isolated active component, disclosed herein (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition. Administration can be, for example, intravenous, transdermal, intramuscular or oral administration. Pharmaceutical compositions can be produced for use in these routes of administration.

A polypeptide, Hbb peptide, fusion protein, conjugate, agent or nucleic acid molecule (including RNA, DNA and a vector), can be formulated for parenteral administration by injection. Compositions for injection may be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. The compositions can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Pharmaceutical compositions can contain antioxidants, buffers, bacteriostats and solutes which render the composition isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. It should be understood that in addition to the ingredients particularly mentioned above, the pharmaceutical compositions described above can include other agents conventional in the art having regard to the type of pharmaceutical composition in question, for example those suitable for oral administration can include flavoring agents.

The pharmaceutical composition can be formulated for extended release, see for example, Nie et al., Biomacromolecules 2021, 22, 2299-2324. Systems for extended release include, but are not limited to, polymer based release systems, protein-based release systems, lipid based release systems, polyphenol-based release systems, and inorganic materials-based release systems.

The pharmaceutical composition may be formulated as a extended release system to permit release of the active ingredient(s) over a specific period of time. A release system can include a matrix of a biodegradable material or a material which releases the incorporated active ingredient(s) by diffusion. The active ingredient(s) can be homogeneously or heterogeneously distributed within the release system. A variety of release systems may be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that active ingredients having different molecular weights are released by diffusion through or degradation of the material.

In some embodiments, liposomes are utilized. In a formulation for injection, the liposome capsule degrades due to cellular digestion. Without being bound by theory, these formulations provide the advantages of an extended-release drug delivery system, exposing a subject to a substantially constant concentration of the active ingredient(s) over time. In one example, the active ingredient(s) can be dissolved in an organic solvent, such as DMSO or alcohol, as previously described, and contain a polyanhydride, poly(glycolic) acid, poly(lactic) acid, or polycaprolactone polymer.

In some embodiments, a nanodispersion system can be utilized, see, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

In some embodiments, a polyester, such as PLGA is utilized, such as in the form of PLGA microparticles or in situ gel/implant formulations. Food and Drug Administration (FDA) approved composition include LUPRON DEPOT®, LUPANETA PACK®, ZOLADEX® Depot, SOMATULINE® Depot, ELIGARD®, and BYDUREON®.

Dendrimers are synthetic three-dimensional macromolecules that are prepared in a step-wise fashion from simple branched monomer units, the nature and functionality of which can be easily controlled and varied. Dendrimers consist of an initiator core, surrounded by a layer of a selected polymer that is grafted to the core, forming a branched macromolecular complex. Dendrimers are typically produced using polymers such as poly(amidoamine) or poly(L-lysine). A dendrimer can be synthesized from the repeated addition of building blocks to a multifunctional core (divergent approach to synthesis), or towards a multifunctional core (convergent approach to synthesis) and each addition of a three-dimensional shell of building blocks leads to the formation of a higher generation of the dendrimers. Polypropylenimine dendrimers contain 100% protonable nitrogens and up to 64 terminal amino groups. Protonable groups are usually amine groups which are able to accept protons at neutral pH. For nucleic acid molecules, dendrimers can be formed from polyamidoamine and phosphorous containing compounds with a mixture of amine/amide or N—P(O2)S as the conjugating units. Dendrimers of use for delivery of nucleic acid molecules is disclosed, for example, in PCT Publication No. 2003/033027.

Unit dosage pharmaceutical compositions are those containing an effective dose, as hereinbelow recited, or an appropriate fraction thereof, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. A pharmaceutical composition of use in the disclosed methos can include an effective amount of a disclosed polypeptide, Hbb peptide, conjugate, agent, fusion protein and/or nucleic acid molecule, in any combination.

The disclosed polypeptides, Hbb peptides, conjugates, agents, fusion proteins and nucleic acid molecules can be effective over a wide dosage range and can be generally administered in an effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

Administration may be provided as a single administration, a periodic bolus (for example, intravenously) or as continuous infusion from an internal reservoir (for example, from an implant disposed at a specific location or from an external reservoir (for example, from an intravenous bag). Administration, such as by injection, can be performed once, or can be performed repeatedly, such as 1, 2, 3, 4, 5, 6, 7, 8, 9, 10, 20, 30, 40, 50, 60, 70, 80, 90, or 100 or more times. Administration can be performed biweekly, weekly, every other week, monthly, or every 2, 3, 4, 5, or 6 months.

Individual doses are typically not less than an amount required to produce a measurable effect on the subject and may be determined based on the pharmacokinetics and pharmacology of the subject composition or its by-products, and thus based on the disposition of the composition within the subject. This includes consideration of the route of administration as well as dosage amount, which can be adjusted for intravenous or intrahepatic applications. Effective amounts of dose and/or dose regimen can readily be determined empirically from preclinical assays, from safety and escalation and dose range trials, individual clinician-patient relationships, as well as in vitro and in vivo assays.

In another embodiment, the dosage is a daily dose. In another embodiment, the dosage is a weekly dose. In a further embodiment, the dosage is twice a day. In other embodiments, the dosage is a biweekly dose, a bimonthly dose, or a monthly dose. In another embodiment, the dosage is an annual dose. In another embodiment, the dose is one is a series of a defined number of doses. In another embodiment, the dose is a on-time dose.

In some embodiments, a disclosed polypeptide, Hbb peptide, conjugate, agent or fusion protein can be administered at a therapeutically effective dose is from about 0.01 g to about 1 g per day. In some example, a therapeutically effective dose is from 1 mg to about 900 mg, such as 10 mg to 900 mg, such as 100 mg to about 900 mg. In further example a therapeutically effective dose is from about 1 mg to about 800 mg, such as about 10 mg to about 800 mg, such as about 100 mg to about 800 mg. In some examples, a therapeutically effective dose is from about 1 mg to about 700 mg, such as about 10 mg to about 700 mg, such as about 100 mg to about 700 mg. In more examples, a therapeutically effective dose is from about 1 mg to about 600 mg, such as about 10 mg to about 600 mg, such as about 100 mg to about 600 mg. In other examples, a therapeutically effective dose is from about 1 mg to about 500 mg, such as about 10 mg to about 500 mg, such as about 100 mg to about 500 mg. In more examples, a therapeutically effective dose is from 1 mg to about 400 mg, such as about 10 mg to about 400 mg, such as about 100 mg to about 400 mg. In yet other examples, a therapeutically effective dose is from about 1 mg to about 300 mg, such as about 10 mg to about 300 mg, such as about 100 mg to about 300 mg. In some examples, a therapeutically effective dose is from about 1 mg to about 200 mg, such as about 10 mg to about 200 mg, such as about 100 mg to about 200 mg. Suitable doses include about 150, 200, 250, 300, 350, 400, 450 and 500 mg. Suitable doses also include 250 to about 450 mg, such as about 250 to about 400 mg, such as about 350 mg. These doses can be daily doses.

In other embodiment, the therapeutically effective dose is about 1 to about 10 mg per kg of body weight. Suitable doses include about 2 to about 10 mg per kg of body weight, about 3 to about 10 mg per kg of body weight, about 4 to about 10 mg per kg of body weight, about 5 mg to about 10 mg per kg of body weight, about 6 mg to about 10 mg per kg of body weight, about 7 to about 10 mg per kg of body weight. Suitable doses also include, for example, about 4 to about 6 mg per kg of body weight, about 4, 5 or 6 mg per kg of body weight.

In some embodiments, one or more polypeptides, Hbb peptides, and/or fusion proteins constitute about 0.01% to about 50% of the pharmaceutical composition. In some embodiments, the peptides constitute about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific non-limiting examples include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition.

With regard to compositions that include mRNA, various embodiments of dosage ranges of mRNA can be used in methods of the present disclosure. In one embodiment, the dosage is in the range of about 0.1 to about 0.9 μg/day. In some embodiments, the dosage range can be about 0.1 to about 0.8 μg/day, about 0.1 to about 0.7 μg/day, about 0.1 to about 0.6 μg/day, about 0.1 to about 0.5 μg/day, about 0.1 to about 0.4 μg/day, about 0.1 to about 0.3 μg/day, or about 0.1 to about 0.2 μg/day. In more embodiments, the dosage range can be about 0.2 to about 0.9 μg/day, about 0.3 to about 0.9 g/day, about 0.4 to about 0.9 g/day, about 0.5 to about 0.9 g/day, about 0.6 to about 0.9 μg/day, about 0.7 to about 0.9 μg/day, or about 0.8 to about 0.9 μg/day. The dose can be about 0.1 μg/day, about 0.2 g/day, about 0.3 μg/day, about 0.4 μg/day, about 0.5 μg/day, about 0.6 μg/day, about 0.7 μg/day, about 0.8 μg/day or about 0.9 μg/day.

In one embodiment, the dosage is in the range of 1-10 μg/day. In another embodiment, the dosage is 2-10 μg/day. In another embodiment, the dosage is 3-10 μg/day. In another embodiment, the dosage is 5-10 μg/day. In another embodiment, the dosage is 2-20 g/day. In another embodiment, the dosage is 3-20 μg/day. In another embodiment, the dosage is 5-20 μg/day. In another embodiment, the dosage is 10-20 g/day. In another embodiment, the dosage is 3-40 μg/day. In another embodiment, the dosage is 5-40 μg/day. In another embodiment, the dosage is 10-40 μg/day. In another embodiment, the dosage is 20-40 μg/day. In another embodiment, the dosage is 5-50 μg/day. In another embodiment, the dosage is 10-50 μg/day. In another embodiment, the dosage is 20-50 μg/day. In one embodiment, the dosage is 1-100 μg/day. In another embodiment, the dosage is 2-100 μg/day. In another embodiment, the dosage is 3-100 μg/day. In another embodiment, the dosage is 5-100 μg/day. In another embodiment the dosage is 10-100 μg/day. In another embodiment the dosage is 20-100 μg/day. In another embodiment the dosage is 40-100 μg/day. In another embodiment the dosage is 60-100 μg/day.

In another embodiment, the dosage is 0.1 μg/day. In another embodiment, the dosage is 0.2 μg/day. In another embodiment, the dosage is 0.3 μg/day. In another embodiment, the dosage is 0.5 μg/day. In another embodiment, the dosage is 1 μg/day. In another embodiment, the dosage is 2 mg/day. In another embodiment, the dosage is 3 μg/day. In another embodiment, the dosage is 5 μg/day. In another embodiment, the dosage is 10 μg/day. In another embodiment, the dosage is 15 μg/day. In another embodiment, the dosage is 20 μg/day. In another embodiment, the dosage is 30 μg/day. In another embodiment, the dosage is 40 μg/day. In another embodiment, the dosage is 60 μg/day. In another embodiment, the dosage is 80 μg/day. In another embodiment, the dosage is 100 μg/day.

In another embodiment, the dosage is 10 g/dose. In another embodiment, the dosage is 20 μg/dose. In another embodiment, the dosage is 30 μg/dose. In another embodiment, the dosage is 40 μg/dose. In another embodiment, the dosage is 60 μg/dose. In another embodiment, the dosage is 80 μg/dose. In another embodiment, the dosage is 100 μg/dose. In another embodiment, the dosage is 150 μg/dose. In another embodiment, the dosage is 200 μg/dose. In another embodiment, the dosage is 300 g/dose. In another embodiment, the dosage is 400 μg/dose. In another embodiment, the dosage is 600 μg/dose. In another embodiment, the dosage is 800 μg/dose. In another embodiment, the dosage is 1000 μg/dose. In another embodiment, the dosage is 1.5 mg/dose. In another embodiment, the dosage is 2 mg/dose. In another embodiment, the dosage is 3 mg/dose. In another embodiment, the dosage is 5 mg/dose. In another embodiment, the dosage is 10 mg/dose. In another embodiment, the dosage is 15 mg/dose. In another embodiment, the dosage is 20 mg/dose. In another embodiment, the dosage is 30 mg/dose. In another embodiment, the dosage is 50 mg/dose. In another embodiment, tie dosage is 80 mg/dose. In another embodiment, the dosage is 100 mg/dose.

In another embodiment, the dosage is 10-20 μg/dose. In another embodiment, the dosage is 20-30 μg/dose. In another embodiment, the dosage is 20-40 μg/dose. In another embodiment, the dosage is 30-60 μg/dose. In another embodiment, the dosage is 40-80 μg/dose. In another embodiment, the dosage is 50-100 μg/dose. In another embodiment, the dosage is 50-150 μg/dose.

In another embodiment, the dosage is 100-200 μg/dose. In another embodiment, the dosage is 200-300 μg/dose. In another embodiment, the dosage is 300-400 μg/dose. In another embodiment, the dosage is 400-600 μg/dose. In another embodiment, the dosage is 500-800 μg/dose. In another embodiment, the dosage is 800-1000 μg/dose. In another embodiment, the dosage is 1000-1500 μg/dose. In another embodiment, the dosage is 1500-2000 μg/dose. In another embodiment, the dosage is 2-3 mg/dose. In another embodiment, the dosage is 2-5 mg/dose. In another embodiment, the dosage is 2-10 mg/dose. In another embodiment, the dosage is 2-20 mg/dose. In another embodiment, the dosage is 2-30 mg/dose. In another embodiment, the dosage is 2-50 mg/dose. In another embodiment, the dosage is 2-80 mg/dose. In another embodiment, the dosage is 2-100 mg/dose. In another embodiment, the dosage is 3-10 mg/dose. In another embodiment, the dosage is 3-20 mg/dose. In another embodiment, the dosage is 3-30 mg/dose. In another embodiment, the dosage is 3-50 mg/dose. In another embodiment, the dosage is 3-80 mg/dose. In another embodiment, the dosage is 3-100 mg/dose. In another embodiment, the dosage is 5-10 mg/dose. In another embodiment, the dosage is 5-20 mg/dose. In another embodiment, the dosage is 5-30 mg/dose. In another embodiment, the dosage is 5-50 mg/dose. In another embodiment, the dosage is 5-80 mg/dose. In another embodiment, the dosage is 5-100 mg/dose. In another embodiment, the dosage is 10-20 mg/dose. In another embodiment, the dosage is 10-30 mg/dose. In another embodiment, the dosage is 10-50 mg/dose. In another embodiment, the dosage is 10-80 mg/dose. In another embodiment, the dosage is 10-100 mg/dose.

The recombinant mRNA can be used alone. However, in another embodiment, at least one additional agent can be included along with the nucleic acid molecule in the composition.

Pharmaceutical compositions including nucleic acid molecules can be formulated for injection, such as, but not limited to, for intravenous or intra-arterial administration. Such compositions are formulated generally by mixing a disclosed nucleic acid molecule at the desired degree of purity in a unit dosage injectable form (solution, suspension, or emulsion) with a pharmaceutically acceptable carrier, for example, one that is non-toxic to recipients at the dosages and concentrations employed and is compatible with other ingredients of the formulation. Pharmaceutical compositions can include an effective amount of the nucleic acid molecule dispersed (for example, dissolved or suspended) in a pharmaceutically acceptable carrier or excipient. Pharmaceutically acceptable carriers and/or pharmaceutically acceptable excipients are known in the art and are described, for example, in Remington's Pharmaceutical Sciences by E. W. Martin, Mack Publishing Co., Easton, PA, 19th Edition (1995). The nature of the carrier will depend on the particular mode of administration being employed. For example, formulations usually contain injectable fluids that include pharmaceutically and physiologically acceptable fluids, such as water, physiological saline, balanced salt solutions, aqueous dextrose, glycerol, or the like, as a vehicle. In addition, pharmaceutical compositions to be administered can contain minor amounts of non-toxic auxiliary substances, such as wetting or emulsifying agents, preservatives, pH buffering agents and the like, for example, sodium acetate or sorbitan monolaurate. A disclosed nucleic acid molecule can be suspended in an aqueous carrier, for example, in an isotonic or hypotonic buffer solution at a pH of about 3.0 to about 8.5, such as about 4.0 to about 8.0, about 6.5 to about 8.5, or about 7.4. Useful buffers include saline-buffered phosphate or an ionic boric acid buffer. The active ingredient, optionally together with excipients, can also be in the form of a lyophilisate and can be made into a solution prior to administration by the addition of suitable solvents.

In some embodiments, the excipients confer a protective effect to a virus including the nucleic acid molecules, such as AAV virion or lentivirus virion, such that loss of AAV virions or lentivirus virions, as well as transduceability resulting from formulation procedures, packaging, storage, transport, and the like, is minimized. These excipient compositions are therefore considered “virion-stabilizing” in the sense that they provide higher virion titers and higher transduceability levels than their non-protected counterparts, as measured using standard assays, see, for example, Published U.S. Application No. 2012/0219528, incorporated herein by reference. These compositions therefore demonstrate “enhanced transduceability levels” as compared to compositions lacking the particular excipients described herein and are therefore more stable than their non-protected counterparts.

Exemplary excipients that can used to protect a virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients in any of the disclosed compositions including virus, such as AAV, varies and is readily determined by one of skill in the art. For example, a protein excipient, such as BSA, if present, will can be present at a concentration of between 1.0 weight (wt.) % to about 20 wt. %, such as 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

A nucleic acid molecule, such as a vector, can be formulated to permit release over a specific period of time. A release system can include a matrix of a biodegradable material or a material which releases the incorporated nucleic acid molecule by diffusion. The nucleic acid molecule can be homogeneously or heterogeneously distributed within the release system. A variety of release systems may be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that active ingredients having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers, and mixtures thereof.

In some embodiments, a therapeutically effective dose will be on the order of from about 10⁵ to 10¹⁶ of virions (such as AAV virions), such as 10⁸ to 10¹⁴ virions. The dose depends on the efficiency of transduction, promoter strength, the stability of the message and the protein encoded thereby, and clinical factors. Effective dosages can be readily established by one of ordinary skill in the art through routine trials establishing dose response curves.

In some embodiments, if the nucleic acid molecule is included in an AAV vector, an effective amount will be about 1×10⁸ vector genomes or more, in some cases about 1×10⁹, about 1×10¹⁰, about 1×10¹¹, about 1×10¹², or about 1×10¹³ vector genomes or more, in certain instances, about 1×10¹⁴ vector genomes or more, and usually no more than about 1×10¹⁵ vector genomes administered to the subject. In some embodiments, the amount of vector that is delivered is about 1×10¹⁴ vectors or less, for example about 1×10¹³, about 1×10¹², about 1×10¹¹, about 1×10¹⁰, or about 1×10⁹ vectors or less, in certain instances about 1×10⁸ vectors, and typically no less than 1×10⁸ vectors administered to the subject. In some non-limiting examples, the amount of vector genomes that is delivered is about 1×10¹⁰ to about 1×10¹¹ vectors. In additional non-limiting examples, the amount of vector that is delivered is about 1×10¹⁰ to about 1×10¹² vector genomes.

In some embodiments, the amount of pharmaceutical composition to be administered may be measured using multiplicity of infection (MOI). In some embodiments, MOI refers to the ratio, or multiple of vector or viral genomes to the cells to which the nucleic may be delivered. In some embodiments, the MOI may be about 1×10⁶. In some cases, the MOI can be about 1×10⁵ to about 1×10⁷. In some cases, the MOI may be about 1×10⁴ to about 1×10⁸. In some cases, recombinant viruses of the disclosure are at least about 1×10¹, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, about 1×10¹⁶, about 1×10¹⁷, and about 1×10¹⁸ MOI. In some cases, recombinant viruses of this disclosure are about 1×10⁸ to 1×10¹⁴ MOI.

In some the amount of pharmaceutical composition delivered comprises about 1×10⁸ to about 1×10¹⁵ particles of recombinant viruses, about 1×10⁹ to about 1×10¹⁴ particles of recombinant viruses, about 1×10¹⁰ to about 1×10¹³ particles of recombinant viruses, or about 1×10¹¹ to about 1×10 s¹² particles of recombinant viruses.

Dosage treatment may be a single dose schedule or a multiple dose schedule to ultimately deliver the amount specified above. Moreover, the subject may be administered as many doses as appropriate. Thus, the recipient may be given, e.g., 10⁵ to 10¹⁶ AAV virions in a single dose, or two, four, five, six or more doses that collectively result in delivery of, e.g., 10⁵ to 10¹⁶ AAV virions. One of skill in the art can readily determine an appropriate number of doses to administer.

In some embodiments, an AAV is administered to the subject at a dose of about 1×10¹¹ to about 1×10¹⁴ viral particles (vp)/kg. In some examples, the AAV is administered at a dose of about 1×10¹² to about 8×10¹³ vp/kg. In other examples, the AAV is administered to the subject at a dose of about 1×10¹³ to about 6×10¹³ vp/kg. In specific non-limiting examples, the AAV is administered to the subject at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the AAV is administered to the subject at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV is administered to the subject at a dose of about 1×10¹² vp/kg. The AAV can be administered in a single dose, or in multiple doses (such as 2, 3, 4, 5, 6, 7, 8, 9 or 10 or more doses) as needed for the desired therapeutic results.

In some embodiments, a lentivirus is administered to the subject at a dose of about 1×10¹¹ to about 1×10¹⁴ viral particles (vp)/kg. In some examples, the lentivirus is administered to the subject at a dose of about 1×10¹² to about 8×10¹³ vp/kg. In other examples, the lentivirus is administered to the subject at a dose of about 1×10¹³ to about 6×10¹³ vp/kg. In specific non-limiting examples, the lentivirus is administered to the subject at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the lentivirus is administered to the subject at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the lentivirus is administered to the subject at a dose of about 1×10¹² vp/kg. The lentivirus can be administered in a single dose, or in multiple doses (such as 2, 3, 4, 5, 6, 7, 8, 9 or 10 or more doses) as needed for the desired therapeutic results.

In some embodiments, the pharmaceutical compositions can be sterilized by conventional sterilization techniques or can be sterile filtered. Aqueous solutions can be packaged for use as is, or lyophilized, the lyophilized preparation being combined with a sterile aqueous carrier prior to administration. In some embodiments, the pH is about 3 to about 11, about 5 to about 9, about 5.5 to about 6.5, or about 5.5 to about 7.5. It will be understood that use of certain of the foregoing excipients, carriers, or stabilizers will result in the formation of pharmaceutical salts.

Methods are disclosed herein for increasing vascular nitric oxide (NO) bioavailability in a subject in need thereof. In some embodiments, the subject can have atherosclerosis. NO synthesized by endothelial nitric oxide synthase (eNOS) plays a role in the maintenance of vascular tone and structure. Decreased vascular NO bioavailability is a feature of cardiovascular diseases (CVD). Without being bound by theory, an impairment of endothelium-dependent vasorelaxation is present in atherosclerotic vessels, and represents the reduced eNOS-derived NO bioavailability. Endothelial dysfunction characterized by an impairment of endothelium dependent vasorelaxation, and reduced eNOS-derived NO bioactivity, is involved in atherogenesis. The increase in vascular NO bioavailability can be compared to a control. The control can be a standard value. The control can be the amount of vascular NO bioavailability prior to treatment. The disclosed methods can be used to increase vascular NO bioavailability, and thus inhibit atherogenesis in a subject.

Methods are also disclosed herein for inhibiting vasoconstriction in a subject. The vasoconstriction can be al adrenoreceptor-mediated vasoconstriction. Therapeutic application of a disclosed composition can be used to provide vasodilation in a subject, such as to hypoxemic and ischemic tissue in the subject. Methods are also disclosed herein for increasing vasodilation in a subject. The inhibition of vasoconstriction, and/or increase in vasodilation, can be compared to a control. The control can be a standard value. The control can be the amount of vasoconstriction or vasodilation, respectively, prior to treatment.

In other embodiments, the method reduces vasoconstriction of resistance vasculature. In further embodiments, the method relaxes resistance arterioles in a subject. The present disclosure additionally provides methods for increasing blood flow to a tissue of a subject. Subjects can be selected for treatment that are in need of reduced vasoconstriction.

In some embodiments, a method is provided for decreasing blood pressure in a subject. These methods include administering an effective amount of a disclosed pharmaceutical composition. The disclosed method can include selecting a subject with elevated blood pressure for treatment. The decrease in blood pressure can be in comparison to a control, such as a standard value or the blood pressure of the subject prior to treatment.

In some embodiments, methods are provided for reducing hypertension in a subject. In some examples, and subject can be selected for treatment that has hypertension. In some embodiments, hypertension, primary hypertension, treatment resistant hypertension, obesity-related hypertension, stroke, myocardial infarction, coronary artery disease, or pulmonary arterial hypertension. The subject can have, for example, elevated blood pressure, hypertension stage 1, hypertension stage 2, or be in a hypertensive crisis. In some embodiments, the subject has elevated blood pressure. In other embodiments, the subject has hypertension stage 1 or hypertension stage 2. In further embodiments, the subject has pulmonary arterial hypertension. The reduction in hypertension can be in comparison to a control, such as a standard value or a measurement from the subject prior to treatment.

In other embodiments, the subject has stroke, myocardial infarction, coronary artery disease, pulmonary arterial hypertension, peripheral arterial disease, congestive heart failure, angina, cerebral artery vasospasm, stroke, transient ischemic attack (TIA), persistent pulmonary hypertension of the newborn, coronary artery vasospasm, Raynaud's phenomenon, erectile dysfunction, acute kidney injury, renal vasoconstriction, pheochromocytoma, malaria, or sepsis. The subject can have angina. These subjects can be selected for treatment.

The disclosed methods can be used to prevent and treat conditions associated with the cardiovascular system, for example, high blood pressure, pulmonary hypertension, cerebral vasospasm and tissue ischemia-reperfusion injury. These discoveries also provide methods to increase blood flow to tissues, for example, to tissues in regions of low oxygen tension.

In further embodiments, the subject has hemolysis or was the recipient of a blood product or substitute. These subjects can be selected for treatment.

Methods are also provided for treating a vascular condition. In some embodiments, the vascular condition is pulmonary hypertension (including neonatal pulmonary hypertension, primary pulmonary hypertension, and secondary pulmonary hypertension), systemic hypertension, cutaneous ulceration, acute renal failure, chronic renal failure, intravascular thrombosis, or an ischemic central nervous system event, such as stroke. In further embodiments, the subject has peripheral vascular disease. In yet other embodiments, the subject has atherosclerosis. These subjects can be selected for treatment.

In some embodiments, the subject has decreased blood flow to a tissue, and the method increases blood flow. the decreased blood flow to the tissue is caused directly or indirectly by at least one of the following conditions: malaria, falciparum malaria, bartonellosis, babesiosis, clostridial infection, severe Haemophilus influenzae type b infection, extensive burns, transfusion reaction, cardiopulmonary bypass, coronary disease, cardiac ischemia syndrome, angina, iatrogenic hemolysis, angioplasty, myocardial ischemia, tissue ischemia, hemolysis caused by intravascular devices, hemolysis caused by medical or genetic conditions, transfusion of blood or hemoglobin or blood substitutes, hemodialysis, pulmonary hypertension, systemic hypertension, cutaneous ulceration, acute renal failure, chronic renal failure, intravascular thrombosis, and an ischemic central nervous system event, such as a stroke. In some embodiments, the subject has ischemia. In further embodiments, the subject has sepsis. In additional embodiments, the method treats or ameliorates hepatic or cardiac or brain ischemia-reperfusion injury. In further embodiments, the disclosed pharmaceutical compositions are of use to treat hypertension, primary hypertension, treatment resistant hypertension, obesity-related hypertension, stroke, myocardial infarction, coronary artery disease, or pulmonary arterial hypertension. These subjects can be selected for treatment.

Also provided in other examples of this embodiment are methods for treating or ameliorating cerebral artery vasospasm. The subject can have a stroke, or a transient ischemic attack. These subjects can be selected for treatment.

The disclosure further provides a method for treating a subject having a condition associated with elevated blood pressure in the lungs, e.g. pulmonary hypertension. In some embodiments, this includes treating a subject having neonatal pulmonary hypertension. In some embodiments, this includes treating a subject having primary and/or secondary pulmonary hypertension. These subjects can be selected for treatment.

The disclosure also provides suggestions for a means of treating hypertension and/or preeclampsia in pregnant women. Such therapy would include action on spastic and diseased blood vessels within the placenta. These subjects can be selected for treatment.

In examples of the methods, the subject can have pulmonary hypertension, systemic hypertension, peripheral vascular disease, trauma, cardiac arrest, general surgery, organ transplantation, cutaneous ulceration, acute renal failure, chronic renal failure, intravascular thrombosis, angina, an ischemia-reperfusion event, an ischemic central nervous system event, and death.

The disclosed compositions also are of use in sports medicine, such as for increasing blood flow to a tissue, and/or increasing exercise performance. The disclosed compositions also are of use for treating erectile dysfunction. In these embodiments, administration can be local or systemic. In these embodiments, subject in need of increased performance is selected for treatment.

Thus, there is provided herein a method for inducing vasodilation and/or increasing blood flow in a subject, which method involves administering to the subject an effective amount of a disclosed pharmaceutical composition for a sufficient period of time to induce vasodilation and/or increase blood flow in the subject.

The subject can be any subject of interest. In some embodiments the subject is mammalian, such as a human subject. In more embodiments, the subject is an adult subject. The subject can be an elderly subject, such as one of more than 65 years of age. In some embodiments, the subject is a neonate.

The pharmaceutical composition can be administered, for example, intraperitoneally, intramuscular, intravascularly, or intraventricularly. However, any route of administration is contemplated, including inhalation, oral, rectal, vaginal, transdermal, intra-arterial, and topical. Administration can be local or systemic.

Combination therapy methods are contemplated, wherein a disclosed pharmaceutical composition is administered in combination with at least one additional agent. By way of non-limiting examples, the additional agent is one or more selected from the list consisting of penicillin, hydroxyurea, butyrate, clotrimazole, arginine, or a phosphodiesterase inhibitor (such as sildenafil).

Kits are also provided. The kit can include a disclosed pharmaceutical composition and an instructional material which describes administering the composition to a subject. In another embodiment, this kit comprises a (e.g., sterile) solvent suitable for dissolving or suspending the composition prior to administering the compound to the subject.

The instructional material includes a publication, a recording, a diagram, a reference to a website, or any other medium of expression which can be used to communicate the usefulness of the kit for effecting alleviation of a specified condition. Optionally, or alternately, the instructional material may describe uses such as for use in sports medicine, increasing exercise performance, or increasing muscle perfusion. The instructional material of the kit of the invention may, for example, be affixed to a container which contains a disclosed composition, be shipped together in a container. Alternatively, the instructional material may be shipped separately from the container with the intention that the instructional material and the compound be used cooperatively by the recipient.

The disclosure is illustrated by the following non-limiting Examples.

### EXAMPLES

In small arteries, constriction of vascular smooth muscle triggers local release of nitric oxide from the adjacent endothelial cell. This feedback vasodilation is a homeostatic mechanism that opposes vasoconstriction. Alpha globin and beta globin are expressed in the endothelium of human resistance arteries, form a complex with endothelial nitric oxide synthase (eNOS) at the myoendothelial junction, and limit the release of nitric oxide triggered by alpha-1-adrenergic stimulation. It was determined that beta globin mimetic peptides could be used to enhance nitric oxide signaling across the myoendothelial junction.

### Example 1

HBA1, HBA2, HBB, and eNOS are Expressed in Blood-Free Human Resistance Artery Tissue

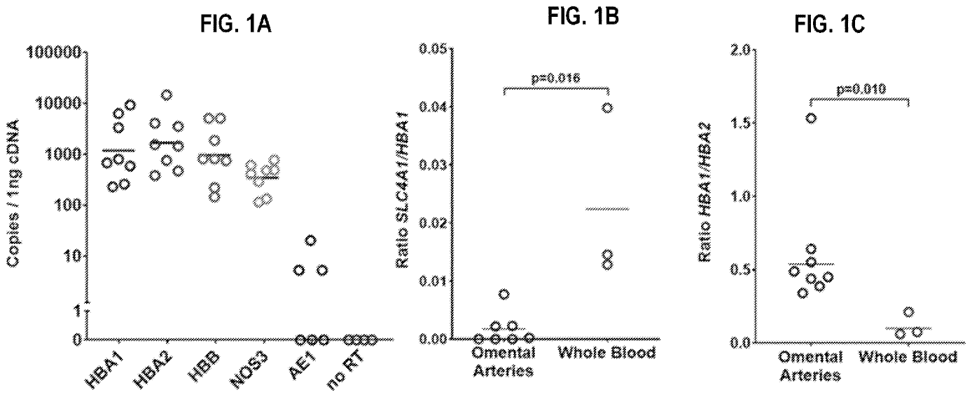

Reverse-transcriptase droplet digital PCR (RT-ddPCR) was used to quantify transcripts from HBA1, HBA2, HBB, and NOS3 in human omental arteries. All four genes were highly expressed in omental arteries (FIG. 1A) and subcutaneous arteries. To assess for potential contamination of arterial tissue with residual blood, the samples were checked for transcripts from an erythroid-specific gene, SLC4A1. SLC4A1 transcripts were abundant in whole blood (geometric mean 4306 transcripts per ng RNA; 95% CI 899, 20638) but very low or absent in omental arteries (2.5; 95% CI 0, 5.2) and subcutaneous arteries (1.8; 95% CI 0, 5.3). Further evidence for artery-specific expression of the globin genes was provided by the unique expression ratios of SLC4A1/HBA1 in arterial tissue versus blood (0.0018±0.0011 vs 0.0224±0.0087; p=0.016; FIG. 1B). Notably, the ratio of HBA1/HBA2 was distinct in arterial tissue compared to whole blood (0.60±0.14 vs 0.12±0.05; p=0.01) (FIG. 1C), raising the possibility that HBA1 and HBA2 are under differential tissue-specific transcriptional control in the artery compared to in the red blood cell progenitor.

### Example 2

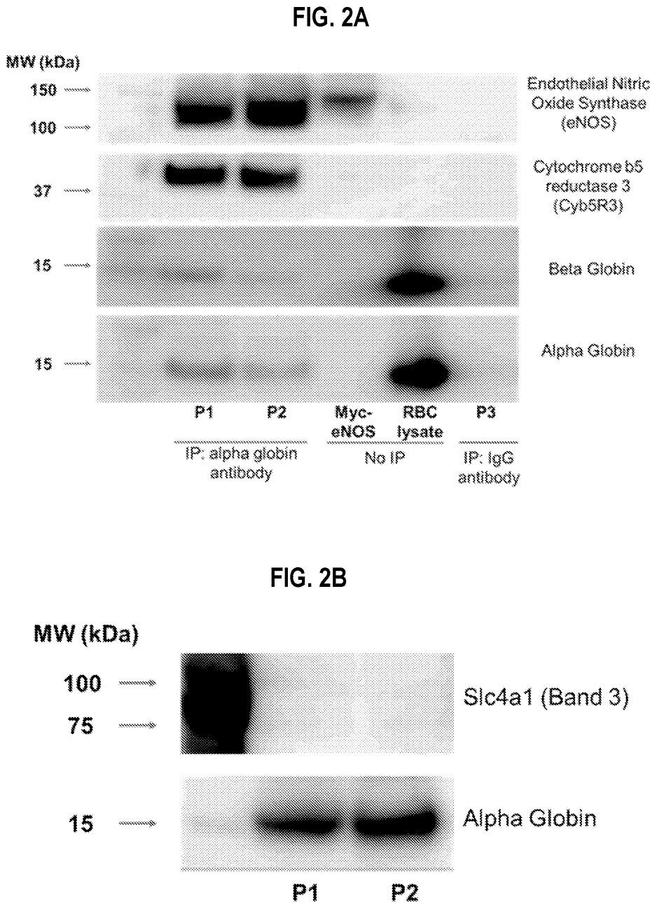

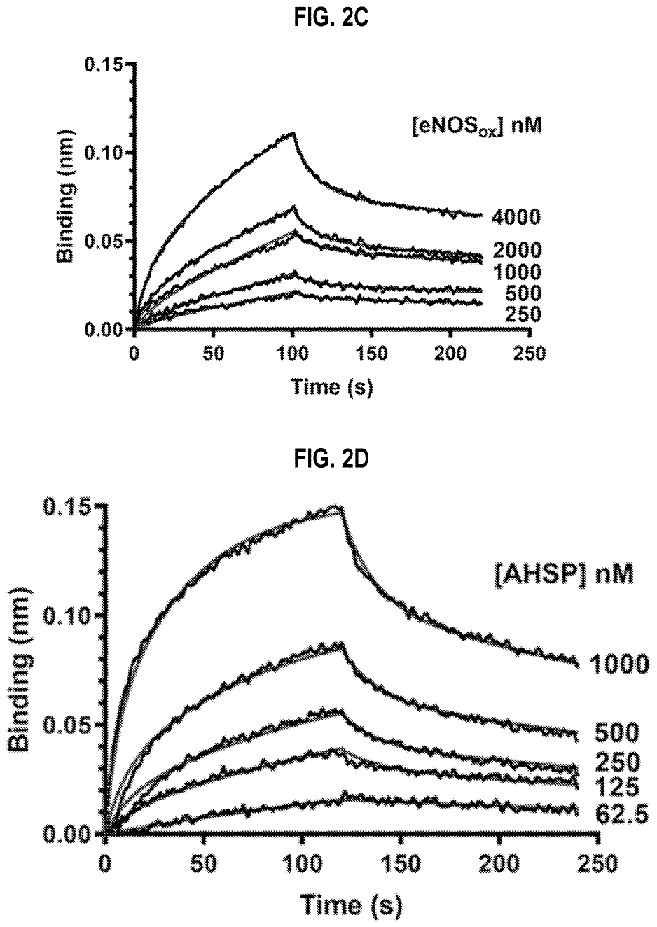

Hemoglobin forms a complex with eNOS in human resistance arteries Based on the observation of FRET between antibodies labeling alpha globin and eNOS, it was determined whether these proteins form a stable complex in vivo. Approximately 30-50 perfused, intact omentum arteries from each of six more donors were homogenized and protein was extracted. Immunoprecipitation was performed with an antibody against alpha globin. Western blot detected alpha globin, beta globin, and eNOS in the anti-alpha globin immunoprecipitate but not in a control immunoprecipitate (FIG. 2A). These results indicate that these three proteins not only co-localize within omental arteries, but also form a stable complex in vivo. To confirm there was no contamination of arterial lysate from residual erythrocytes, Western blot was performed on the lysate with an antibody for SLC4A1, which is the most abundant non-globin protein in the erythrocyte proteome (Bryk and Wisniewski, J Proteome Res. 16:2752-2761, 2017). Even at maximal exposure, no SLC4A1 was detected in pooled arterial lysates (FIG. 2B). Based on previously published work that cytochrome B5 reductase 3 (Cyb5R3) functions as a hemoglobin reductase in rodent models (Straub et al., Nature 491:473-477, 2012; Rahaman et al., J Biol Chem. 290:16861-16872, 2015), samples were probed for Cyb5R3 in the alpha globin immunoprecipitate, and it was found that Cyb5R3 is present in perfused human artery lysates and bound to alpha globin.

Modeling studies had previously predicted alpha globin to interact with the oxidase domain of eNOS (Straub et al., Arterioscler Thromb Vasc Biol. 34:2594-2600, 2014). To test this prediction experimentally, the binding of recombinant human eNOS oxidase domain (eNOSₒₓ) to purified human alpha globin was investigated using bio-layer interferometry (FIG. 2C). eNOSₒₓ bound to immobilized alpha globin with moderate affinity (K_{D}=1.31×10⁻⁶±1.35×10⁻⁷ M; FIG. 2C). For comparison, alpha hemoglobin stabilizing protein bound to alpha globin with stronger affinity (K_{D}=1.09×10⁻⁷±6.50×10⁻⁹ M; FIG. 2D). This biophysical analysis of the binding of alpha globin to eNOS corroborates the FRET and co-immunoprecipitation data supporting the observation of an eNOS-hemoglobin complex.

### Example 3

Alpha Globin and Beta Globin Co-Localize with eNOS at the Myoendothelial Junctions of Human Omental Arteries

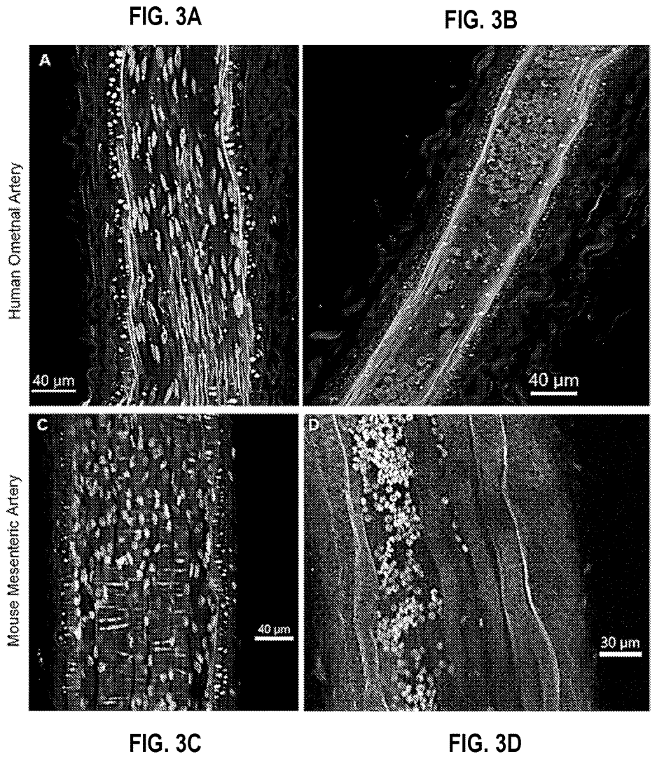

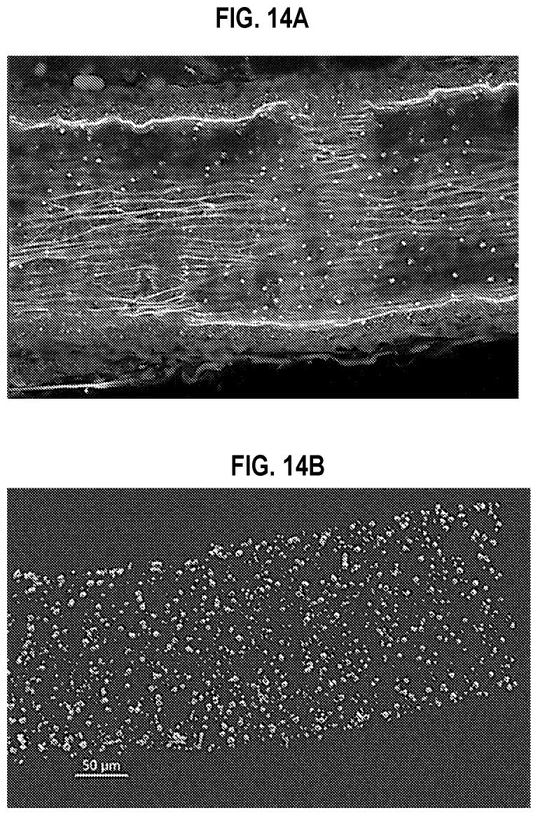

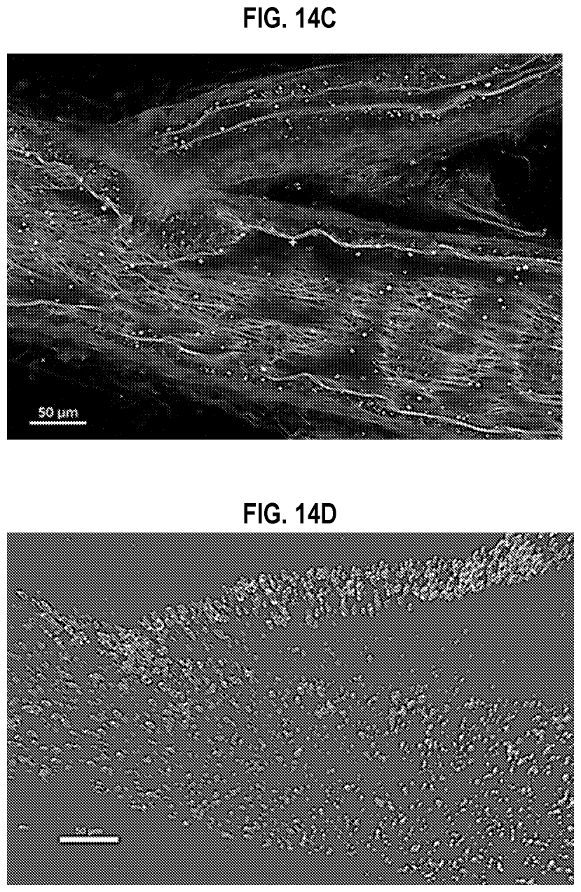

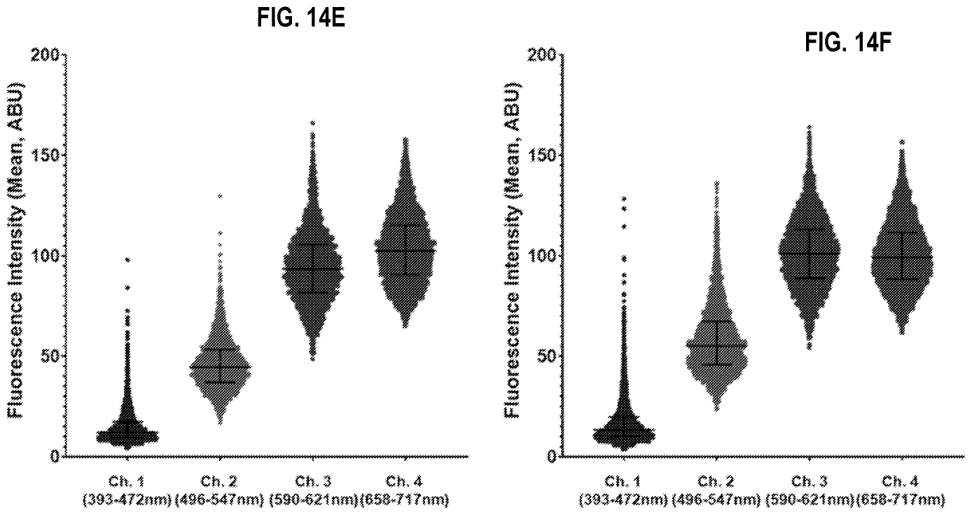

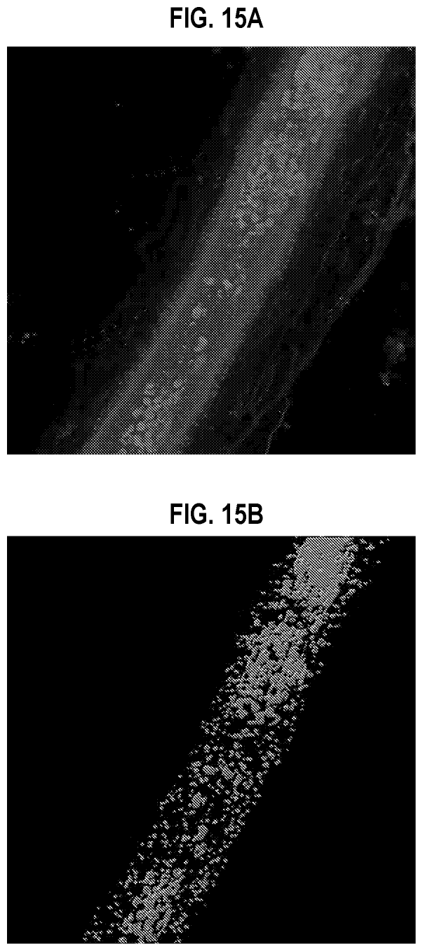

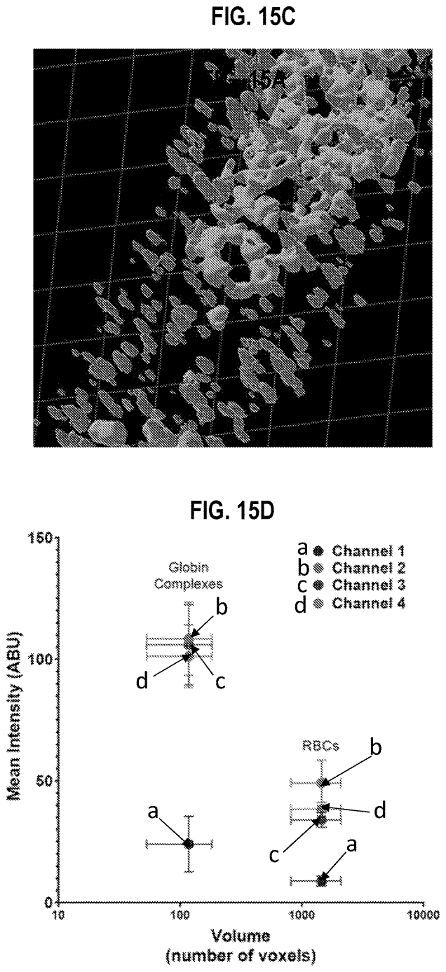

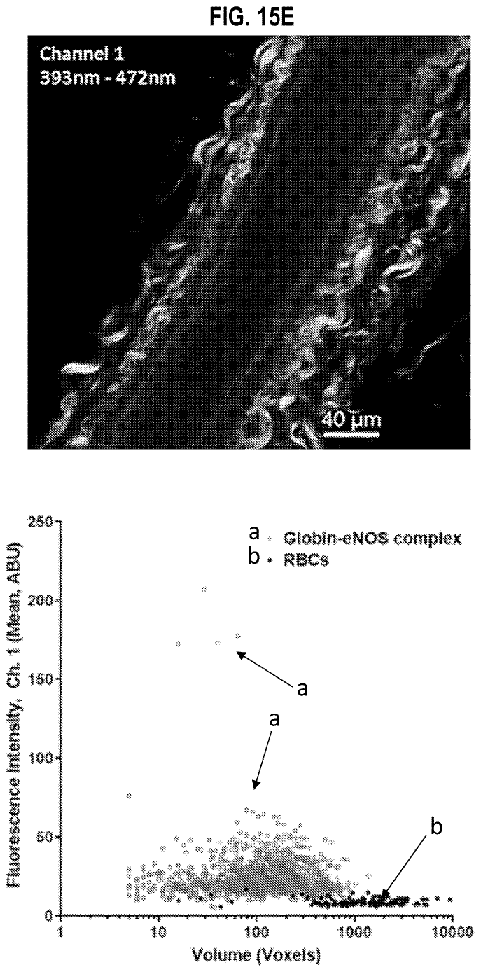

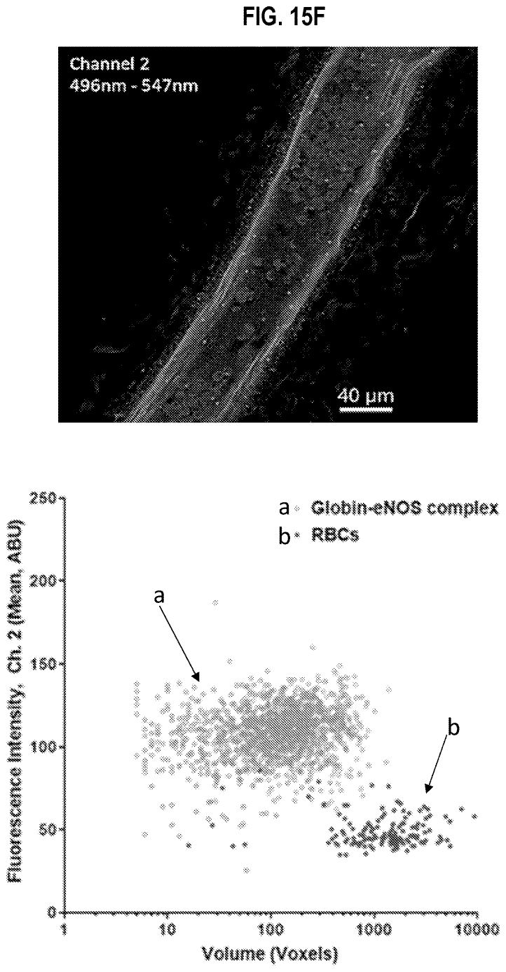

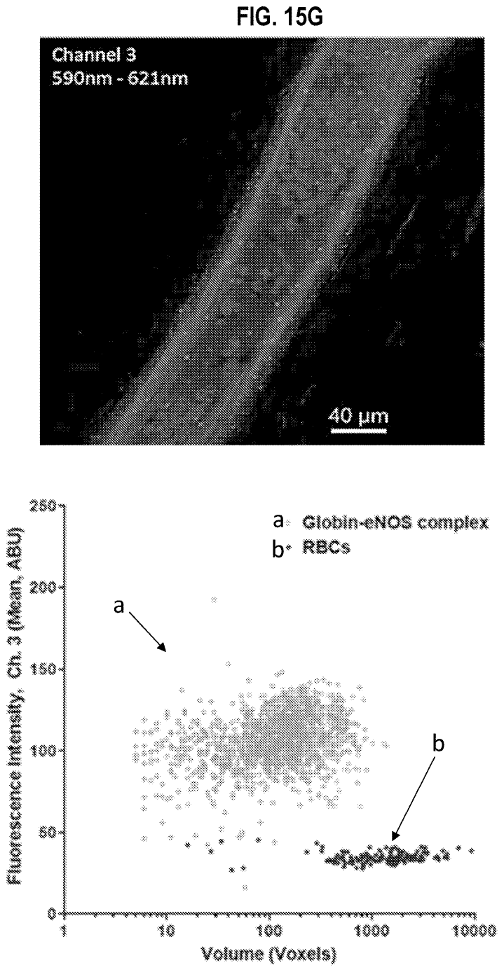

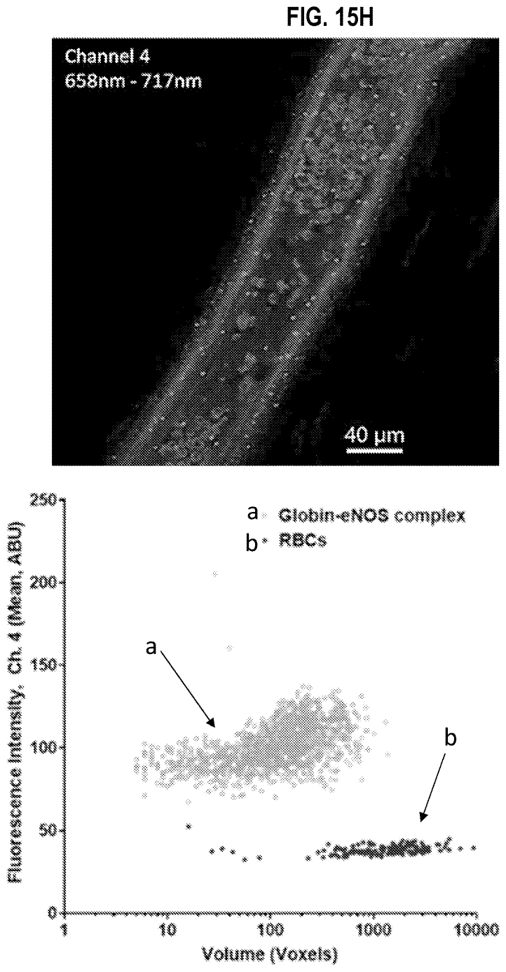

To examine the sub-cellular localization of alpha and beta globin proteins with eNOS in resistance arteries, immunofluorescent multiphoton microscopy was performed on intact human omental artery segments. Multiphoton imaging revealed distinct punctates distributed in the wall of the artery (FIG. 3A) that exhibited broad autofluorescent properties (FIG. 14A-14F). The broad autofluorescence of these punctates is similar to the those of hemoglobin under MP excitation (Zheng et al., Biomed Opt Express. 2:71-79, 2011) (FIG. 15A-D). The autofluorescent properties of the arterial wall punctates were compared against the autofluorescence of hemoglobin within intact red blood cells (RBCs) in the same field of view (FIG. 3B), and it was discovered that the arterial wall punctates were distinct from the RBCs; they were smaller in volume and had greater signal intensities than the RBCs across all channels (FIG. 15E-15H).

A mesenteric artery from a mouse, which had been perfused, fixed, and imaged using the same methodology, was imaged to determine whether eNOS-alpha globin complexes in murine arteries would exhibit similar autofluorescence despite only expressing alpha globin and not beta globin in the arterial wall. Within the murine mesenteric artery, no autofluorescent punctates were observed in the arterial wall (FIG. 3C); however, the internal elastic lamina (IEL) was autofluorescent in a similar pattern to human omental arteries, and the outer collagen layer had a similar second harmonic generation (SHG). A murine mesenteric artery that had not been fully perfused of RBCs was imaged to see if murine hemoglobin within red cells was autofluorescent (FIG. 3D). The murine RBCs within the mesenteric artery demonstrated similar autofluorescence to human RBCs within an omental artery, while no autofluorescent punctates were visible within the wall of the murine mesenteric artery.

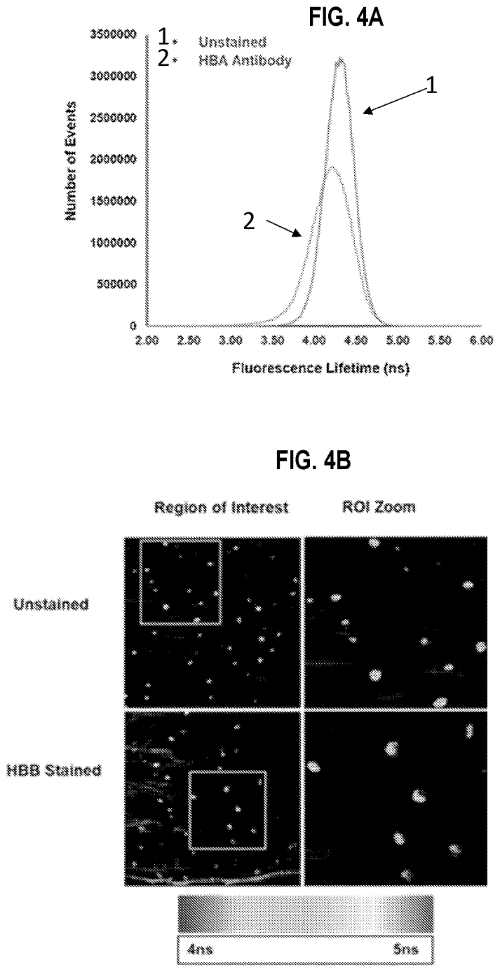

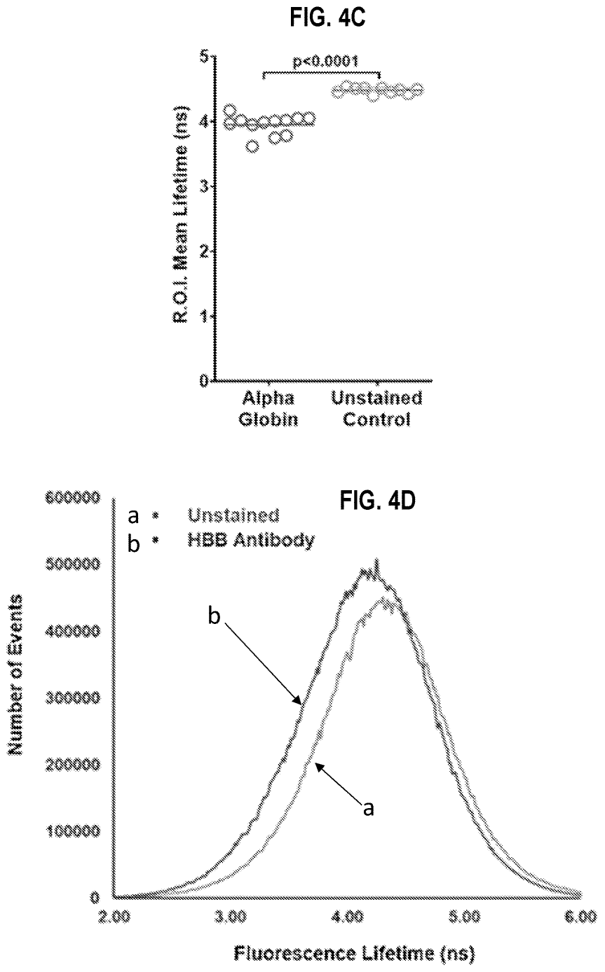

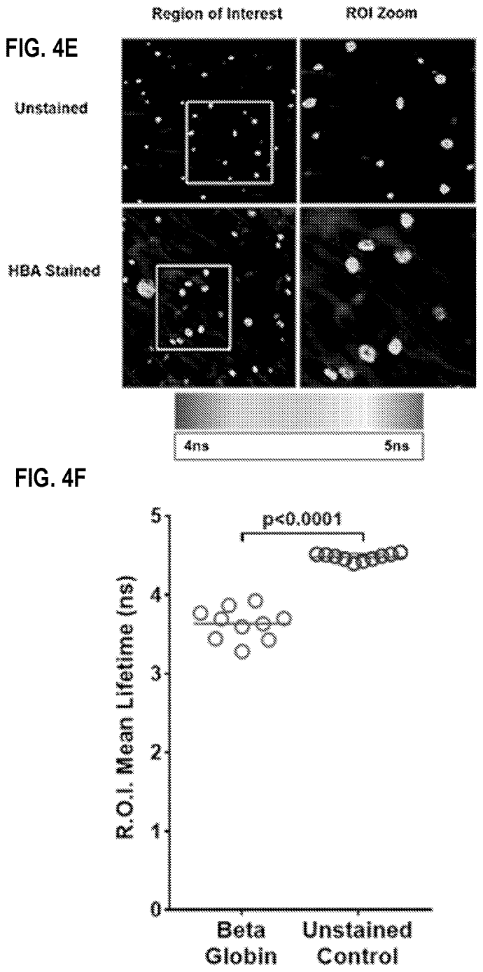

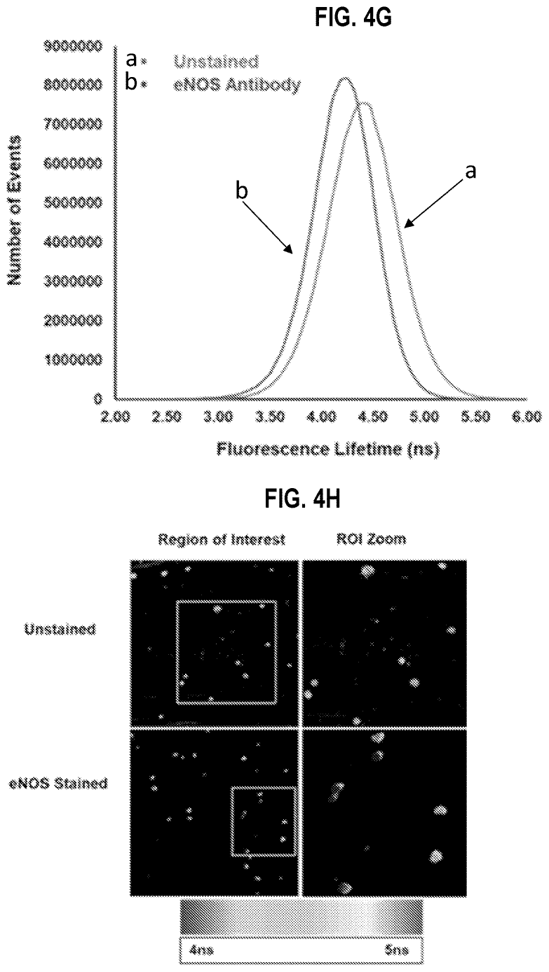

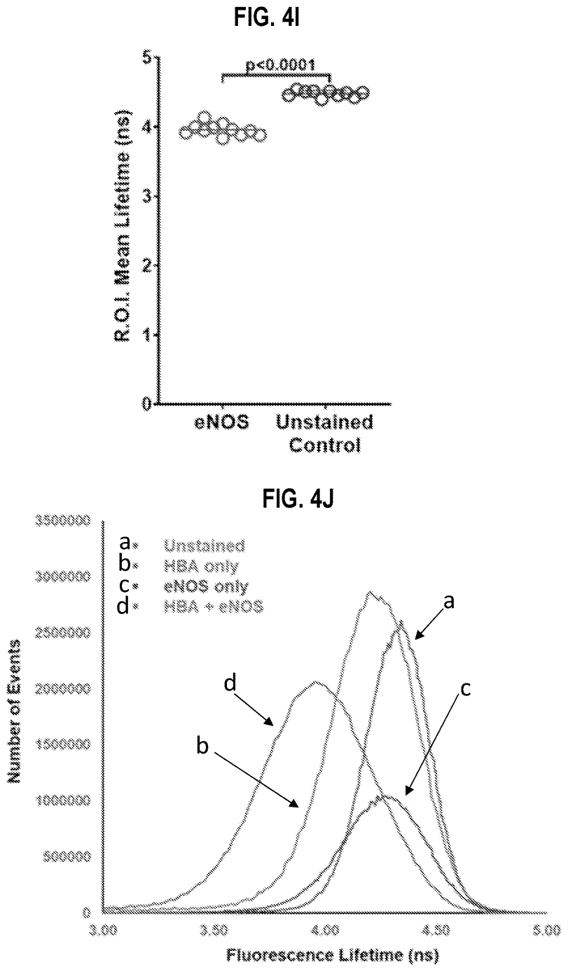

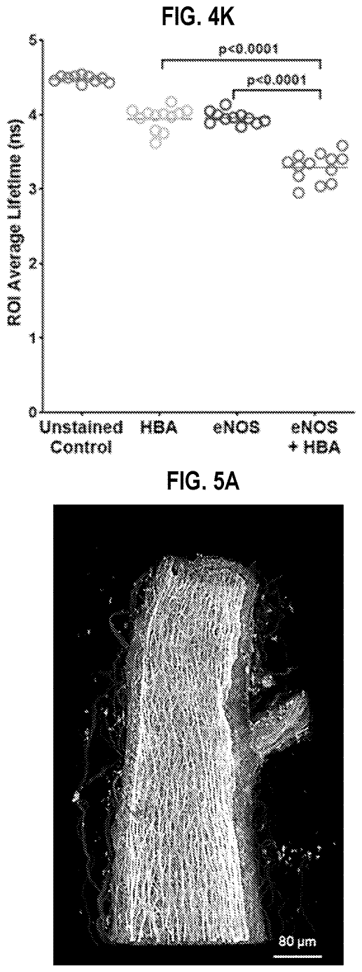

To investigate the protein constituents of this autofluorescent complex, the unique fluorescence lifetimes, rather than the wavelengths, of the antibody-conjugated fluorophores were used to distinguish the autofluorescent punctate from antibodies binding individually to each of alpha globin (FIG. 4 A-C), beta globin (FIG. 4 D-F), and eNOS (FIGS. 4G-4I). The close physical proximity of alpha globin and eNOS was further evaluated by co-immunostaining alpha globin and eNOS with fluorophore conjugated antibodies and assessing for FRET (FIGS. 4J, 4K). The fluorescence lifetime of singly labeled alpha globin (3.95±0.04 ns) and eNOS (3.96±0.02 ns) shifted to a shorter lifetime when co-immunostained (3.29±0.05 ns; p<0.0001), indicating a FRET effect that occurs when two fluorophores are within 10 Angstroms of each other. Together, these observations were consistent with a multi-protein complex that contains alpha globin, beta globin, and eNOS, which comprises the broadly autofluorescent punctate visible in the arterial wall.

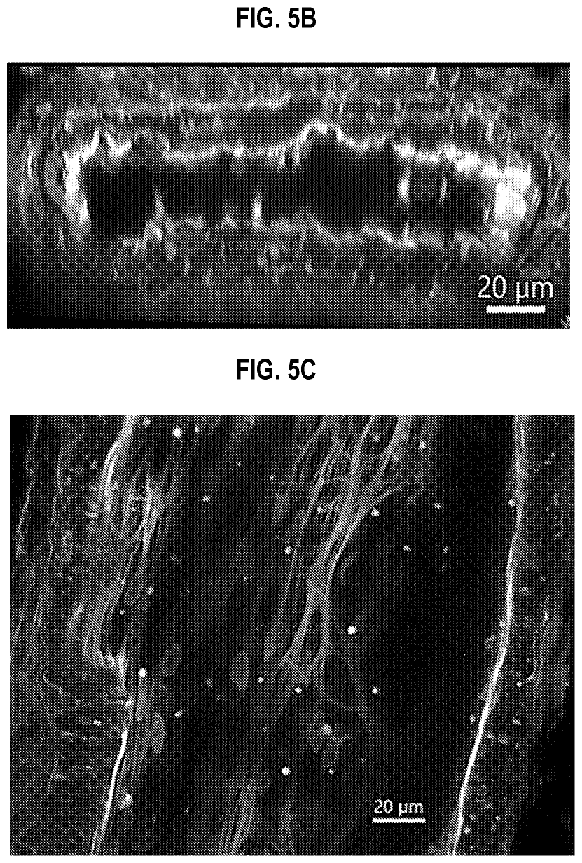

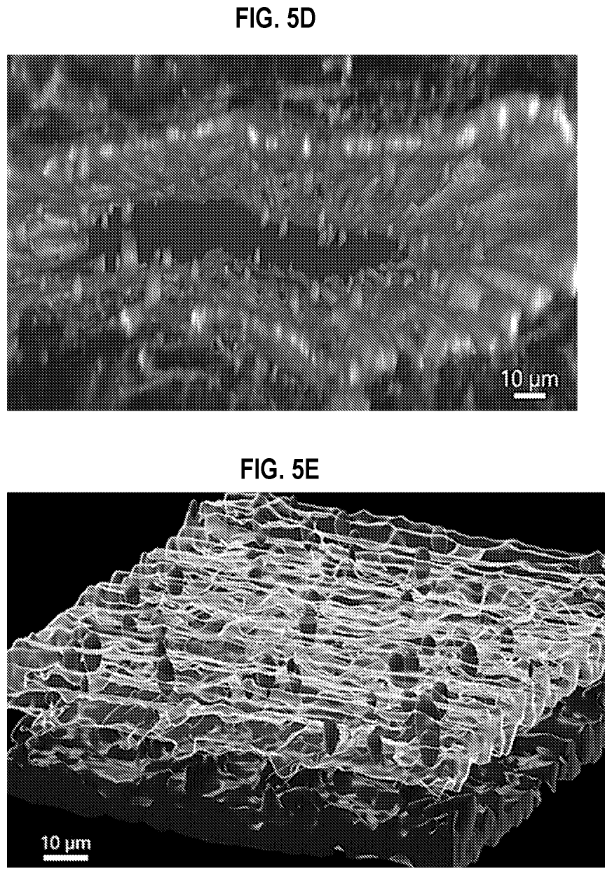

To identify the localization of the hemoglobin-eNOS complexes, whole intact arteries were imaged by multiphoton microscopy (FIG. 5A). The eNOS-hemoglobin complexes localized to the plane of the internal elastic lamina (IEL) as seen in a transverse view (FIG. 5B). They are in closer proximity to the DAPI-stained endothelial cell nuclei (oriented in the direction of flow) than to the vascular smooth muscle nuclei (oriented perpendicular to flow). A longitudinal view reveals the eNOS-hemoglobin complexes to be in the same 1 μm plane as the IEL (FIG. 5C); viewing the artery in three dimensions, the pink punctates are visible protruding through and on the endothelial side of the IEL (FIG. 5D), A 3-D computer reconstruction (Imaris Bitplane) of an arterial segment identifies the eNOS-hemoglobin complexes to occupy regions of the endothelial cell that traverse the IEL (FIG. 5E); these regions are consistent with the size and location of myoendothelial junctions.

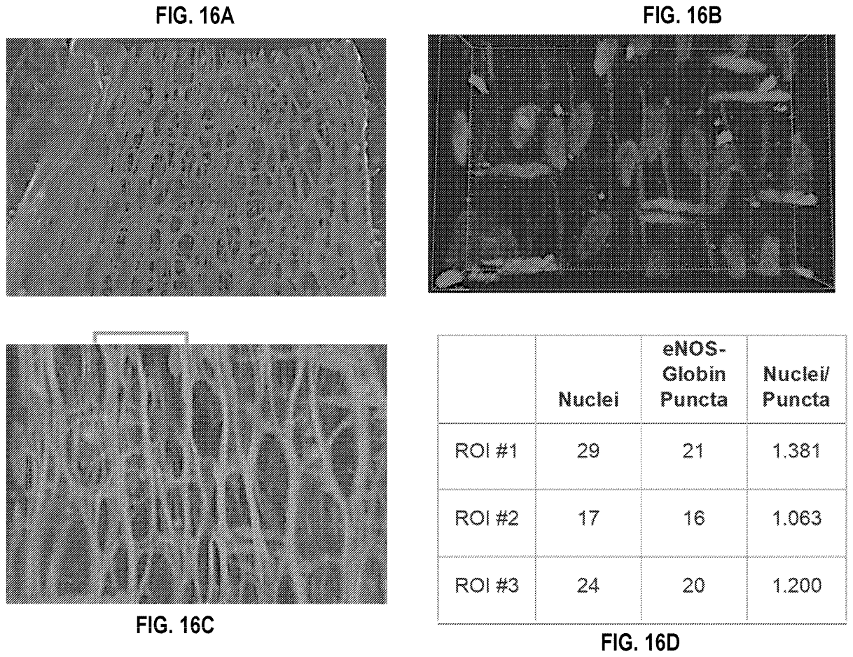

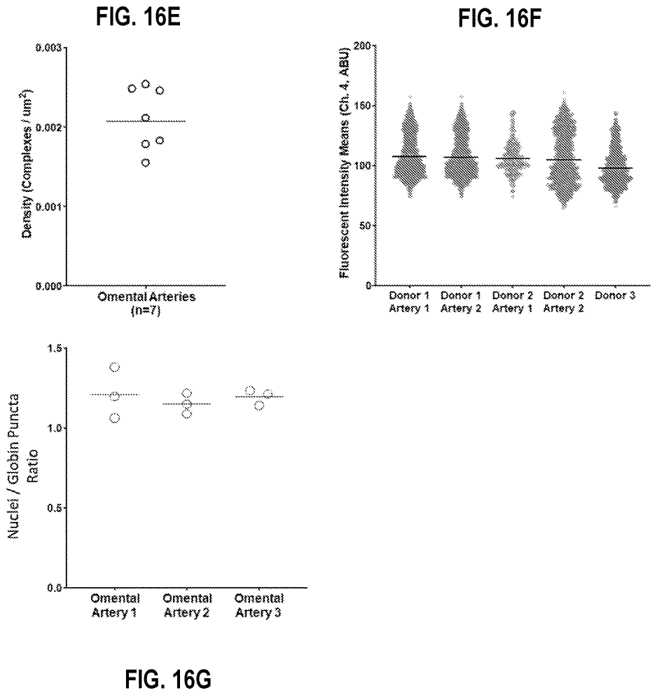

It was next observed that each endothelial cell appeared to have a single subcellular domain containing the eNOS-hemoglobin complexes (FIGS. 16A-16D). The ratio of autofluorescent punctates to endothelial cell nuclei was consistent across artery segments from three different donors (1.21, 1.15, and 1.20), providing a mean±SEM of 1.19±0.03 (FIG. 16G). The spatial density of eNOS-hemoglobin-containing punctates was calculated by counting the number of punctates per arterial surface area (FIG. 16E). The mean density was 0.0021±0.0004 complexes/μm² and was consistent across seven donors. To investigate whether fluorescent intensities of the eNOS-hemoglobin-containing punctates were consistent between two different arteries taken from the same donor, five arteries collected from three omental tissue donors were imaged. Arteries from the same donor did not differ in intensity, but one donor out of three had lower signal intensities than the other two (FIG. 16F and the Table below).

Table: Four parameter logistic regression best-fit values for artery pressure myography performed on pairs of omental arteries from 5 human donors.

### Example 4

Disruption of the eNOS-Hemoglobin Complex Enhances Feedback Vasodilation to an Alpha-Adrenergic Agonist in Human Omental Resistance Arteries

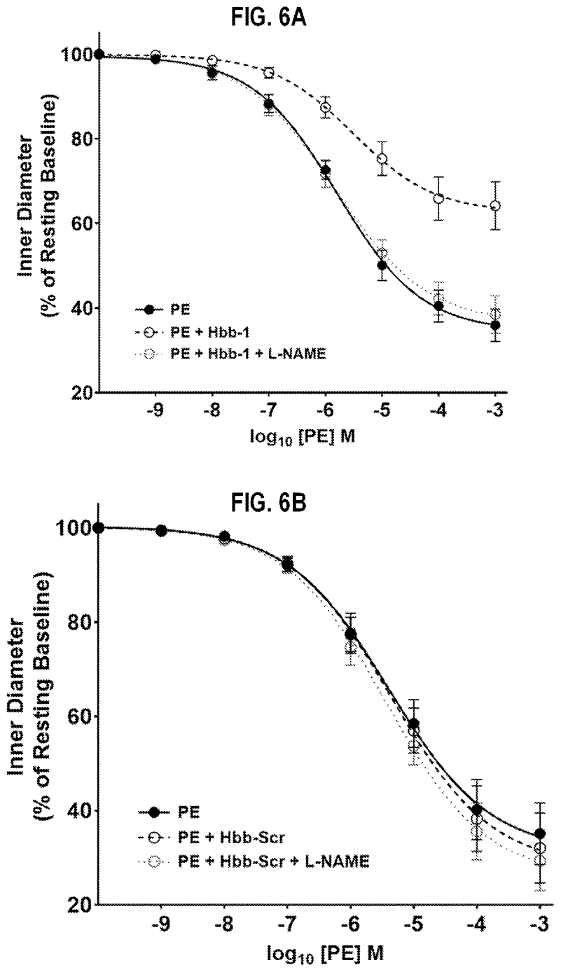

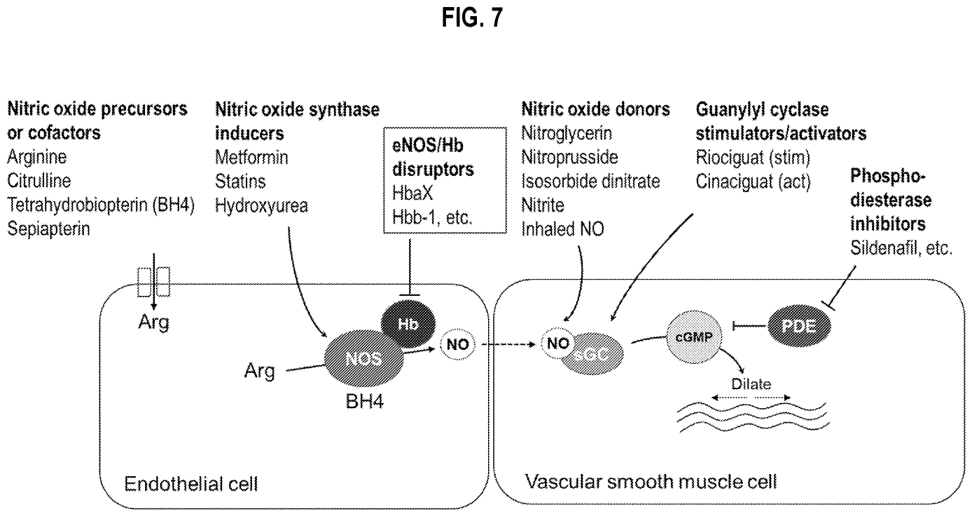

It was hypothesized that disruption of the hemoglobin/eNOS complex would acutely enhance feedback vasodilation from NO released from eNOS at the MEJ in response to alpha-1-adrenergic stimulation because the displacement of hemoglobin would render it less effective at scavenging NO produced by eNOS. To test this hypothesis, ex vivo pressure myography was performed on pairs of omental arteries obtained from each of five donors in response to escalating doses of the alpha adrenoreceptor agonist phenylephrine (responses fit with four parameter logistic regression best-fit values, FIGS. 6A-6B).

Initially, omental arteries in each pair maximally constricted to phenylephrine to a similar degree (4-PL minimum, 39.1±3.2% vs 46.1±5.5% of baseline diameter; p=0.30; FIGS. 6 A,B).

| Mimetic Peptide | Top | | Bottom | | LogIC50 | | HillSlope | |
|---|---|---|---|---|---|---|---|---|
| (n = 5 human donors) | Mean | SEM | Mean | SEM | Mean | SEM | Mean | SEM |
| Phenylephrine (PE) | 100.12 | 2.14 | 39.08 | 3.17 | −5.891 | 0.15 | −0.568 | 0.108 |
| PE + Mimetic | 99.93 | 0.83 | 64.59 | 1.55 | −5.575 | 0.12 | −0.549 | 0.076 |
| PE + Mimetic + | 100.58 | 1.19 | 41.89 | 2.02 | −5.768 | 0.10 | −0.497 | 0.054 |
| LNAME | | | | | | | | |
| Control Peptide | | | | | | | | |
| (n = 5 human donors) | Mean | SEM | Mean | SEM | Mean | SEM | Mean | SEM |
| Phenylephrine (PE) | 100.30 | 4.04 | 46.11 | 5.49 | −6.000 | 0.31 | −0.602 | 0.241 |
| PE + Control | 100.40 | 4.13 | 45.78 | 5.50 | −6.020 | 0.31 | −0.623 | 0.256 |
| PE + Control + | 100.40 | 3.36 | 45.40 | 4.55 | −6.021 | 0.25 | −0.582 | 0.187 |
| LNAME | | | | | | | | |

Next, in one vessel from each donor pair, vasoconstriction was measured after incubation with the alpha globin mimetic peptide HbαX, which was previously established to disrupt binding between the alpha subunit of hemoglobin and eNOS (Straub et al., Nature 491:473-477, 2012; Straub et al., Arterioscler Thromb Vasc Biol. 34:2594-2600, 2014). The second vessel from each donor pair was treated with a control peptide before phenylephrine responses were measured. Arteries exposed to the alpha globin mimetic peptide maximally constricted less than those exposed to the control peptide (64.6±1.6% vs 39.1±3.2%; p<0.01). To determine whether the enhanced feedback vasodilation was NOS-dependent, each artery was incubated with a combination of the peptide plus the NOS inhibitor L-NAME, and vasoconstriction to PE measured a third time. Following the incubation of mimetic peptide-treated arteries with L-NAME, phenylephrine-induced vasoconstriction was restored to 41.9±2.0% of baseline (p<0.0001 vs mimetic peptide alone), no different from the initial phenylephrine-induced response (p=0.53). L-NAME had no effect on the arteries treated with the control peptide (p=0.99). Thus, disruption of the hemoglobin-eNOS complex enhanced feedback vasodilation to alpha-1-adrenergic stimulation in a NOS-dependent manner.

Blood pressure and blood flow are regulated by the dynamic and homeostatic behavior of resistance arteries. A major vasoconstrictive signal, alpha-1-adrenergic stimulation of vascular smooth muscle, is counterbalanced by the coupled release of vasodilatory signals such as nitric oxide (NO) from the adjacent endothelial cell (Dora et al., Proc Natl Acad Sci USA. 94:6529-6534, 1997; Garland et al., Sci Signal. 10:3806, 2017; Hong et al., Arterioscler Thromb Vase Biol. 38:542-554, 2018). In 2012, Straub et al proposed a novel vasoregulatory mechanism whereby this coupled release of NO from endothelial cells is limited by monomeric alpha globin (Straub et al., Nature 491:473-477, 2012). That mechanism was based primarily on studies of cultured cells and murine arteries, but had yet to be systematically evaluated in the human arterial context.

The disclosed studies elucidate the expression, binding partners, sub-cellular localization, and vasoregulatory function of alpha globin in human resistance arteries.

Human resistance arteries were dissected from fresh omental tissue, individually cannulated and perfused to remove blood, and then subjected to a range of molecular, biochemical, imaging, and functional studies. This systematic study, carried out entirely in the context of human resistance arteries, provided insight into normal human arterial function. The data presented herein suggest that hemoglobin, not alpha globin alone, regulates eNOS-dependent NO signaling between endothelium and smooth muscle of human resistance arteries.

Evidence supporting the discovery of hemoglobin in the human resistance artery wall came from multiple orthogonal approaches. First, endogenous expression of the alpha globin (HBA1, HBA2) and beta globin (HBB) genes were detected in blood-free arterial tissue. It was found that all three genes were expressed at a level similar to endothelial nitric oxide synthase (NOS3), a major endothelial gene. This result is in contrast to a study of thoracodorsal arteries from mice that detected HBA but not HBB transcripts (Lechauve et al., J Clin Invest. 128:5073-5082, 2018), and there have been no studies of globin gene expression within perfused human arteries for comparison. To exclude the possibility that these alpha and beta globin transcripts originated from residual blood cells in the perfused arteries, Band3 (SLC4A1) was examined, a gene expressed predominantly in red cell progenitors. SLC4A1 transcripts were very low or absent from each arterial specimen; moreover, the ratio of HBA1:SLC4A1 was much higher in arterial tissue than in whole blood, indicating that the HBA1 transcripts could not have originated from blood contamination. Interestingly, the ratio of HBA1:HBA2 was also greater in arterial tissue compared to blood, suggesting that the alpha globin genes are under endothelial cell-specific transcriptional control that differs from that of red blood cell progenitors. From these studies it was concluded that both alpha and beta globin genes are expressed in cells that make up the wall of human resistance arteries.

Next, the proteins were identified that interact with alpha globin in the context of its role in the arterial wall. In order to obtain sufficient amounts of protein to analyze by Western blot, more than 200 arteries individually cannulated, perfused, and lysed, from multiple donors and pooled them for protein analysis. Both eNOS and beta globin co-immunoprecipitated with alpha globin, implying that these three proteins form a stable complex in vivo. This result is in contrast to prior studies in mouse arteries where eNOS, but not beta globin, co-immunoprecipitated with alpha globin (Straub et al., Nature 491:473-477, 2012). The present results are also in contrast to a study that examined a small number of human subcutaneous adipose arteries by Western blot without co-immunoprecipitation and concluded that alpha globin, but not beta globin, was present (Keller et al., Hypertens Dallas Tex 1979 68.6:1494-1503, 2016). A third study detected more beta globin than alpha globin on a Western blot of protein extracted from human pulmonary arteries, but beta globin was not recognized to be of vascular origin (Alvarez et al., Am J Respir Cell Mol Biol. 57:733-744, 2017).

To further examine the biophysical interactions between alpha globin and eNOS, biolayer interferometry was used, and it was found that these molecules interact with moderate affinity in solution. The equilibrium dissociation constant of full-length alpha globin and the eNOS oxygenase domain had not previously been quantified, but it was similar to that previously reported for an alpha globin-derived peptide and eNOS oxygenase domain (Keller et al., Hypertens Dallas Tex 1979 68.6:1494-1503, 2016). Further evidence for the interaction of alpha globin with eNOS in vivo was provided by the observation of FRET between fluorophore-labeled antibodies against alpha globin and eNOS in situ in the arterial wall. Together, these three orthogonal approaches support the novel observation that alpha globin, beta globin, and eNOS interact and exist as a multi-protein complex in the human omental artery wall.

Cyb5R3, an enzyme that regulates the oxidative state of hemoglobin, was determined to co-immunoprecipitate with alpha globin in protein lysates from perfused human resistance arteries. Cyb5R3 reduces the heme iron from the ferric (Fe³⁺) state to the ferrous (Fe²⁺) state which has a higher affinity for NO; thus Cyb5R3 could be an important redox regulator of nitric oxide signaling by the hemoglobin-eNOS complex (Straub et al., Nature 491:473-477, 2012; Gladwin and Kim-Shapiro, Nature. 491:344-345, 2012).

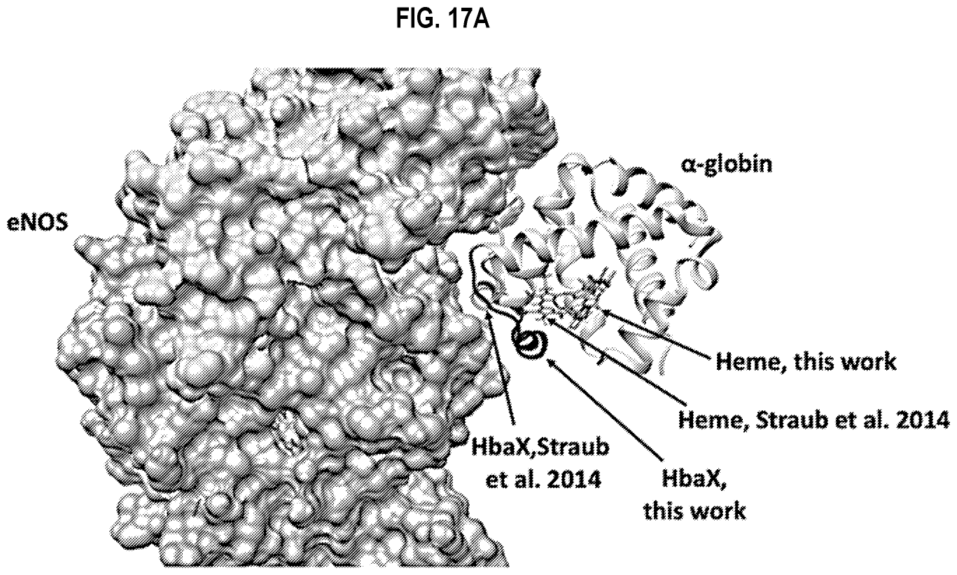

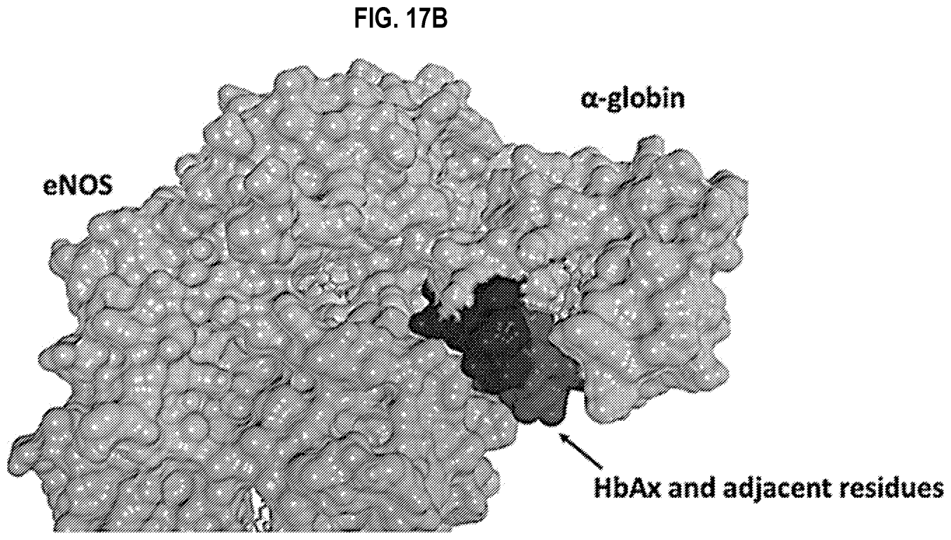

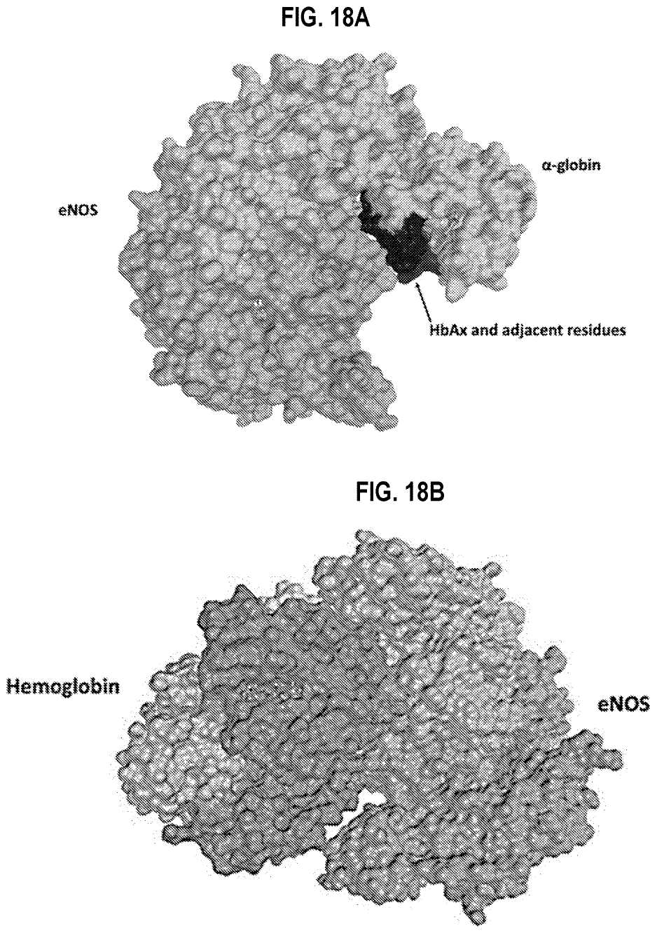

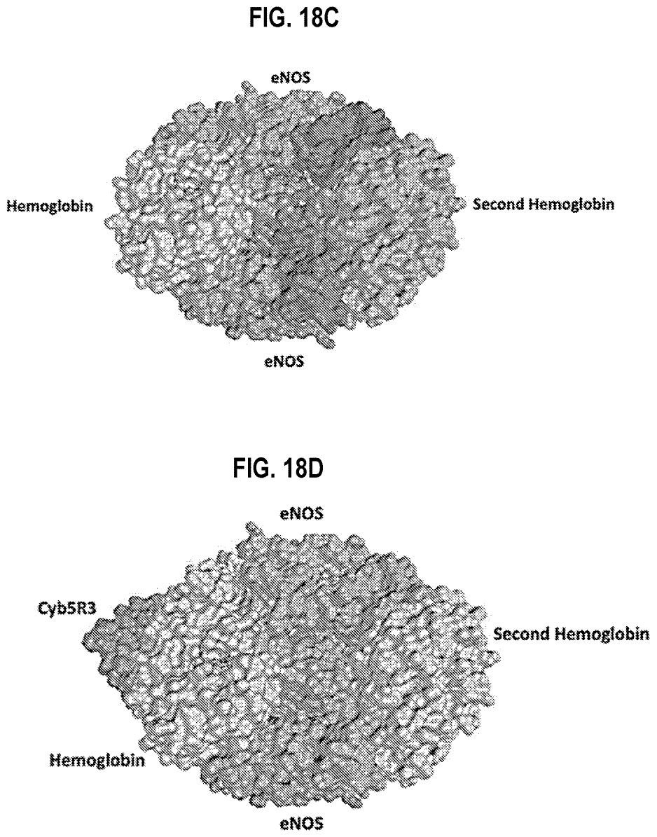

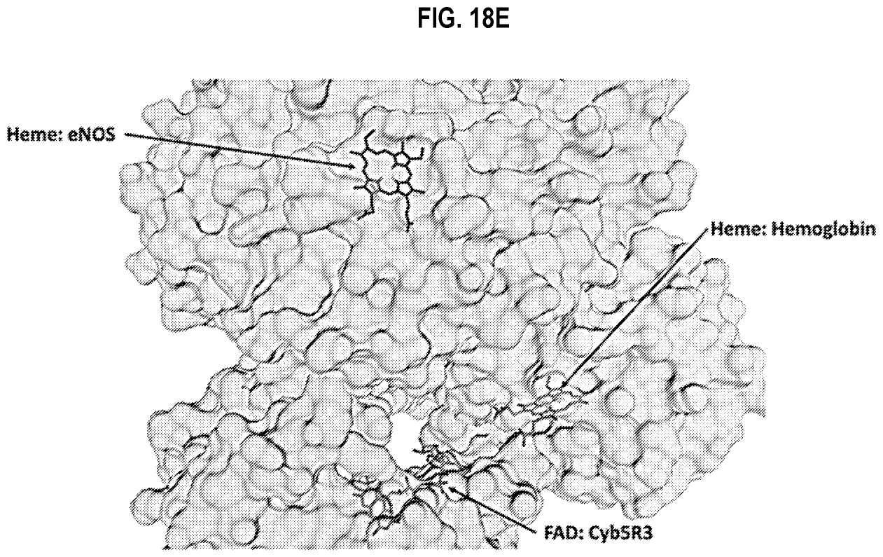

Molecular modeling of eNOS, alpha globin, beta globin and Cyb5R3 accommodates a heterotetrameric hemoglobin molecule bound to an eNOS oxygenase homodimer. The predicted interface between alpha globin and eNOS shifts relative to the model that incorporated an alpha globin monomer alone (FIG. 17A) (Straub et al., Nature 491:473-477, 2012; Straub et al., Arterioscler Thromb Vase Biol. 34:2594-2600, 2014). The revised model identifies novel interfaces between beta globin and eNOS, as well as between beta subunits from different hemoglobin tetramers (FIGS. 18A-18C). The Cyb5R3 FAD binding domain can be brought in close proximity to both the alpha globin heme and eNOS reaction center to potentially facilitate the redox regulation of the alpha globin heme and its ability to scavenge nitric oxide (FIG. 18E).

To understand the sub-cellular localization of this complex within the artery wall, multiphoton (MP) microscopy was employed. The primary advantage of this technique was that it enabled high-resolution imaging of intact arteries without sectioning; a secondary advantage was the ability of MP excitation to highlight structural features of the artery wall such as collagen (via SHG signal) and elastin (via narrow band autofluorescence). A third and unanticipated feature of MP microscopy was the excitation of hemoglobin to produce a broad-spectrum autofluorescence. Distinct, small punctates of intense autofluorescence were observed in the wall of human arteries consistent with the emissions from tetrameric hemoglobin, but the same technique did not reveal autofluorescent punctates in mouse mesenteric arteries, agreeing with multiple prior observations that the beta chain of hemoglobin is absent in mouse arteries (Straub et al., Nature 491:473-477, 2012; Lechauve et al., J Clin Invest. 128:5073-5082, 2018). In a non-perfused artery, these punctates were distinguishable from red blood cells by their size, intensity, and location. It was verified that this autofluorescent complex contained alpha globin, beta globin, and eNOS by measuring the fluorescence lifetimes of photons emitted from fluorophore-labeled antibodies that were distinct from the lifetimes of photons emitted from autofluorescent hemoglobin. This hemoglobin-eNOS complex localized to a small region of each endothelial cell that penetrated the internal elastic lamina, a location that is consistent with the anatomical structure of the myoendothelial junction. This localization is in agreement with previous studies that identified an eNOS-alpha globin complex at the myoendothelial junction in co-cultured vascular cells and in the mouse thoracodorsal artery wall (Straub et al., Nature 491:473-477, 2012). Intact human resistance arteries have not previously been imaged for alpha globin or beta globin for comparison. However, a prior study that employed immunohistochemical analysis of mesenteric artery sections on a human tissue microarray did not detect beta globin (Lechauve et al., J Clin Invest. 128:5073-5082, 2018). The localization of hemoglobin-eNOS to the myoendothelial junction of intact arteries fits with hemoglobin's emerging role as a regulator of directed nitric oxide signaling between endothelium and smooth muscle in human resistance arteries.

It was hypothesized that the contribution of NO during feedback vasodilation, whereby MEJ-localized eNOS produces NO to counteract vasoconstriction, would be regulated by the hemoglobin bound to eNOS. To stimulate eNOS at the MEJ, an alpha-1-adrenergic agonist, phenylephrine, known to activate eNOS at the MEJ via calcium and/or IP₃ signals conducted from vascular smooth muscle, was used (Dora et al., Proc Natl Acad Sci USA. 94:6529-6534, 1997; Garland et al., Sci Signal. 10:3806, 2017; Hong et al., Arterioscler Thromb Vasc Biol. 38:542-554, 2018). Using isolated vessel pressure myography, it was observed that human omental arteries constricted consistently to phenylephrine; however, treatment with an alpha globin mimetic peptide previously established to disrupt binding between eNOS and alpha globin (Straub et al., Arterioscler Thromb Vasc Biol. 34:2594-2600, 2014; Keller et al., Hypertens Dallas Tex 1979 68.6:1494-1503, 2016) diminished this constriction. In the final phase of the myography experiments, the alpha globin mimetic peptide HbαX was applied in combination with a NOS inhibitor and it was determined that the ability of the mimetic peptide to enhance feedback vasodilation was dependent on the enzymatic activity of NOS. This experiment provided further evidence that hemoglobin regulates the diffusion of NO produced directly from eNOS in response to alpha-1-adrenergic vasoconstriction in human resistance arteries.

Together, these observations reinforce that endothelial alpha globin regulates nitric oxide signaling by binding to eNOS at the MEJ (Straub et al., Nature 491:473-477, 2012; Straub et al., Arterioscler Thromb Vasc Biol. 34:2594-2600, 2014). In human arteries, hemoglobin was found bound to eNOS in a complex at the myoendothelial junction where it limits the diffusion of nitric oxide produced enzymatically by eNOS. Studies conducted with the HbαX alpha globin mimetic peptide reveal it to be a potent disruptor of NO-scavenging in human vessels, implying that binding between alpha globin and eNOS is important even when beta globin is present.

Thus, it was determined that hemoglobin forms a complex with eNOS at myoendothelial junctions in human omental resistance arteries where it limits the diffusion of NO produced by eNOS in response to vasoconstriction by an alpha-1-adrenergic stimulus.

### Example 5

### Materials and Methods for Examples 1-4

Collection of omental arteries: Human omental tissue was collected from patients during clinically indicated abdominal operations at the NIH Clinical Center. All patients provided informed consent for tissue procurement on IRB-approved protocol 13-C-0176 (NCT01915225). All tissue removed by the surgeon was immediately submerged in cold 4° C. Krebs-HEPES (KH) buffer (pH 7.4) and kept on ice during dissection and artery isolation. Small arteries were dissected away from the tissue, cannulated on one end with a glass micropipette, and gently perfused with cold KH buffer to remove RBCs from the vessel lumen. Arteries were then prepared for downstream application.

Gene expression studies: Gene expression was measured by reverse-transcriptase droplet digital PCR (ddPCR) using primer/probe assays for HBA1, HBA2, HBB, NOS3, and SLC4A1 (Bio-Rad) in arteries from human omental tissue and human subcutaneous adipose tissue in RNAlater, and human whole blood in PaxGene RNA tubes. Gene expression was quantified as total transcripts of target gene per 1 ng of cDNA.

Co-Immunoprecipitation and Western blot: Protein was extracted (MINUTE™ Total Protein Extraction Kit for Blood Vessels, InventBiotech #SA-03-BV) from perfused small omental arteries from nine individual donors (30-50 arteries each). For Western blot, 20 g of total blood vessels proteins were separated by SDS-PAGE and immunoblotted with anti-Band 3 polyclonal antibody (1:1000, Fisher Scientific #PA5-80030). HRP signal was detected by enhanced chemiluminescence (Pierce).

Co-immunoprecipitation of alpha globin, beta globin, and eNOS was performed on 600 μg of blood vessels lysates pooled from two individual donors. Immunoprecipitation (IP) was performed with alpha globin polyclonal antibody (HBA, 1:1000, Proteintech, #14537-1-AP) or normal rabbit IgG antibody (Cell signaling #2729). Proteins were bound to magnetic beads and pelleted by a magnet stand. Samples were then analyzed by SDS-PAGE and Western blot with the IP antibody, beta globin polyclonal antibody (1:1000, Proteintech #16216-1-AP), eNOS polyclonal antibody (1:1000, Cell Signaling #32027), and cytochrome B5 reductase 3 (Cyb5R3) monoclonal antibody (Abcam abl33247).

Protein-protein binding affinity assessed by biolayer interferometry: Biolayer interferometry experiments were performed on the automated eight-channel Octet RED96 instrument (ForteBio). Alpha globin was biotinylated using EZ-Link™ NHS-PEG4 Biotinylation Kit (Thermofisher Scientific) and immobilized onto Streptavidin (SA) Biosensors. Binding kinetics and data traces were obtained using Data Analysis software v8.2 (ForteBio).

Molecular modeling of alpha globin, hemoglobin, eNOS, and cytochrome B5 reductase: Molecular modeling, graphics, and analysis were produced using UCSF Chimera package (Sanner et al, Biopolymers. 38:305-320, 1996) and the virtual reality mode of UCSF ChimeraX (Pettersen et al., Protein Sci. 30:70-82, 2021). Protein-protein docking was performed using the HADDOCK 2.4 online server (van Zundert et al., J Mol Biol. 428:720-725, 2016). Computational docking of eNOS to alpha globin performed using HADDOCK was guided by specifying the residues from the HbαX peptide on alpha globin and the general area of eNOS that contacted alpha globin on the original model (Straub et al., Arterioscler Thromb Vasc Biol. 34:2594-2600, 2014). Specific conservative substitutions (SEQ ID NO: 7, SEQ ID NO: 8, SEQ ID NO: 9, SEQ ID NO: 10, SEQ ID NO: 11, SEQ ID NO: 12) were determined from analysis of critical residues in the docked model and mutation free energy calculations using the Cartesian AAG tool within the Rosetta software suite (Park et al., J. Chem. Theory Comput. 12, 6201-6212, 2016).

Antibody labeling of intact omental arteries: Isolated and perfused small omental arteries were fixed in 4% formaldehyde in PBS solution (ImageIT, Invitrogen. Vessels undergoing immunofluorescent labeling were permeabilized with 0.1% Triton-X 100 and incubated overnight at 4° C. with primary conjugated antibodies for alpha globin (Abcam ab215919), beta globin (SantaCruz, sc-21757-AF546), or eNOS (SantaCruz, sc-376751-AF594). DAPI (Invitrogen) labeling was performed to demark cell nuclei. Vessels undergoing immunofluorescent labeling with multiple antibodies were permeabilized, blocked in goat serum, and incubated overnight at 4° C. with primary antibodies for alpha globin (Abcam ab92492), beta globin (Santa Cruz sc-21757), and/or eNOS (Abcam ab76198). Arteries were then washed and stained with secondary fluorescent antibodies.

Multiphoton imaging of intact omental arteries: Images were acquired using a Leica SP8 Inverted DIVE (Deep In Vivo Explorer) multiphoton (MP) microscope (Leica Microsystems, Buffalo Grove, IL) with 25.0× and 40.0× Water Immersion Objective, as previously described (Shannon et al, Intravital Imaging of Vaccinia Virus-Infected Mice. In: Mercer J, ed. Vaccinia Virus: Methods and Protocols. New York, NY: Springer; 301-311, 2019). Multiphoton excitation was performed at 880 nm (MaiTai DeepSee, Spectra Physics) and 1150 nm (InSight DeepSee, Spectra Physics) and emitted fluorescence was measured using a 4Tune 4 HyD (4 tunable non-descanned hybrid detectors (HyDs)) reflected light detector. Multiphoton images were collected as a Z-stack in 1-3 μm steps for up to 50 single phases. The collected image files were exported into Huygens Pro SVI and Imaris Bitplane for image deconvolution, processing, and analysis.

Fluorescence lifetime imaging microscopy (FLIM): Images were acquired using the Leica SP8 Inverted DIVE multiphoton microscope. FLIM images were collected using multiphoton excitation with Mai Tai-MP laser (Spectra Physics) tuned at 880 nm at a 80 Mhz frequency and images were simultaneously acquired for MP imaging and FLIM using 4-Tune External Hybrid detectors. Images were acquired at 512-512-pixel format, collecting in excess of 2,000 photons per pixel. Fluorescent Lifetime Decays and Förster resonance energy transfer (FRET/FLIM) efficiency transients and FRET-FLIM images were collected, analyzed, and processed using LASX Single Molecule detection analysis software.

Multiphoton image processing and analysis of fluorescence signal intensity: The Leica Image File (.lif) for each artery was deconvolved to increase resolution and decrease noise and background (Huygens Pro, SVI). Region of interests (ROI) were created and analyzed for signal intensity, density, and volume in Imaris (Bitplane). Mean fluorescence intensity in each detector channel was plotted for each surface object, as well object size in voxels. The density of globin complexes was defined as number of surface objects within the ROI.

Alpha globin mimetic peptide: A previously published molecular modeling study identified a conserved 10 amino acid sequence LSFPTTKTYF (SEQ ID NO: X) that was predicted to facilitate binding of alpha globin to eNOS (Straub et al., Arterioscler Thromb Vasc Biol. 34:2594-2600, 2014). This sequence was combined with an N-terminal HIV-tat tag sequence (YGRKKRRQRRR (SEQ ID NO: Y)) to provide membrane permeability (YGRKKRRQRRRLSFPTTKTYF (SEQ ID NO:Z), Anaspec). This mimetic peptide, called HbαX, has been previously patented for its therapeutic potential. A scrambled version of the peptide with an N-terminal HIV-tat tag sequence (YGRKKRRQRRRFPYFSTKLTT (SEQ ID NO: A), Anaspec) was used as a peptide treatment control.

Isolated artery pressure myography: The pressure myography methodology utilized here for omental arteries is consistent with published protocols for assessing reactivity of mesenteric articles using a DMT pressure myograph (Shahid and Buys, JoVE J Vis Exp. 7.76:50328, 2013). Arteries 100-200 m in diameter were dissected from omental tissue on ice and transferred to the culture myograph wells (DMT-USA CM204); arterial inner diameter was measured by video microscope (DMT-USA) with digital calipers (MyoVIEW, DMT-USA).

All arteries used developed at least 20% myogenic tone after pressurization, consistent with standards for pressure myography (Shahid and Buys, JoVE J Vis Exp. 7.76:50328, 2013; Butcher et al., JoVE J Vis Exp. 28.62:3674, 2012; Jadeja et al., JoVE J Vis Exp. 6.101:e50997, 2015). Arteries were assessed for vasoconstriction response to the alpha-1-adrenoreceptor agonist phenylephrine (PE) [10⁻⁹-10⁻³] M (Sigma-Aldrich). Dose response to PE was then assessed after incubation with either the alpha globin mimetic peptide HbαX or a control peptide (5 μmol/L in KH buffer), and again following incubation with the NOS inhibitor No-Nitro-L-arginine methyl ester HCl (L-NAME, 10⁻⁴ M)(Sigma-Aldrich).

### Example 6

### Determination of Hbb Sequences

For modeling methods, the structural model of this signaling complex was revised by adding beta globin to the model and by refining where alpha globin interacts with eNOS based on this new information. The model structure of Straub, et al. (2014) (Straub et al., Arterioscler Thromb Vasc Biol. 34:2594-2600, 2014) with eNOS and a-globin was used as the basis for modeling with a hemoglobin tetramer. This was accomplished by overlaying one a-globin chain from hemoglobin onto the a-globin from the earlier model using the Matchmaker utility within the UCSF Chimera application. This initial pose between eNOS and hemoglobin was then refined by using the HADDOCK protein-protein docking program. Only interactions between a-globin and eNOS were specified in the input to HADDOCK so as not to bias the program to include eNOS interactions with the beta chain. The resulting models were then analyzed for complementarity of global fit between eNOS and hemoglobin, and similarity of the active a-globin mimetic peptide to its position in the earlier model. Only one cluster of structures met all these criteria, and the best scoring member of this cluster was used in further analysis.

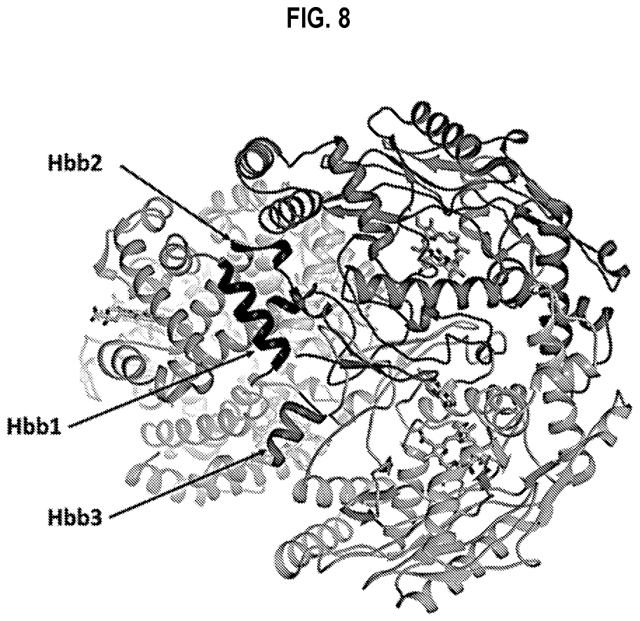

This model suggested refined novel targets for mimetic peptides or small molecules to disrupt Hb-eNOS interactions. Three regions on b-globin were identified that make significant contact with eNOS as candidates for mimetic peptides, see FIG. 8.

Hbb-Peptide-1, b-globin residues 4-17:
(SEQ ID NO: 4)
TPEEKSAVTALWGK
Hbb-Peptide-2, b-globin residues 117-126:
(SEQ ID NO: 5)
HFGKEFTPPV
Hbb-Peptide-3, b-globin residues 87-97:
(SEQ ID NO: 6)
TLSELHCDKLH

These were synthesized with C-terminal tat sequence YGRKKRRQRRR (SEQ ID NO: 14) to enhance cell permeability. Testing with Hbb-Peptide-1 demonstrated that it has potent vasodilatory effects on human arteries ex vivo.

### Hbb-Peptide-1

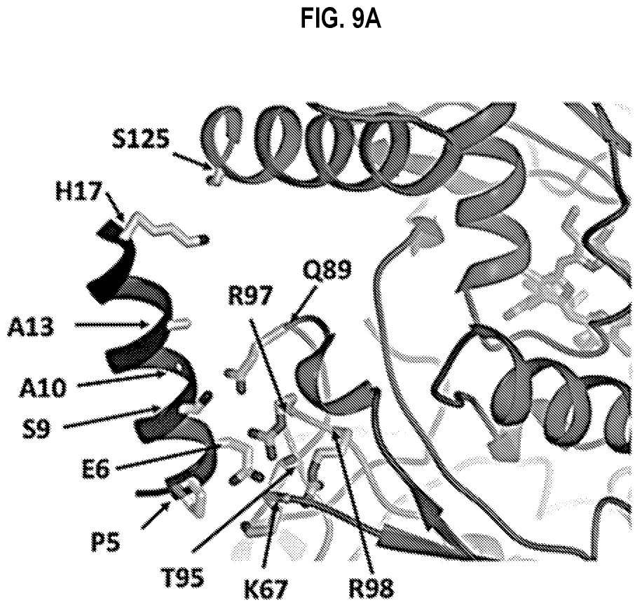

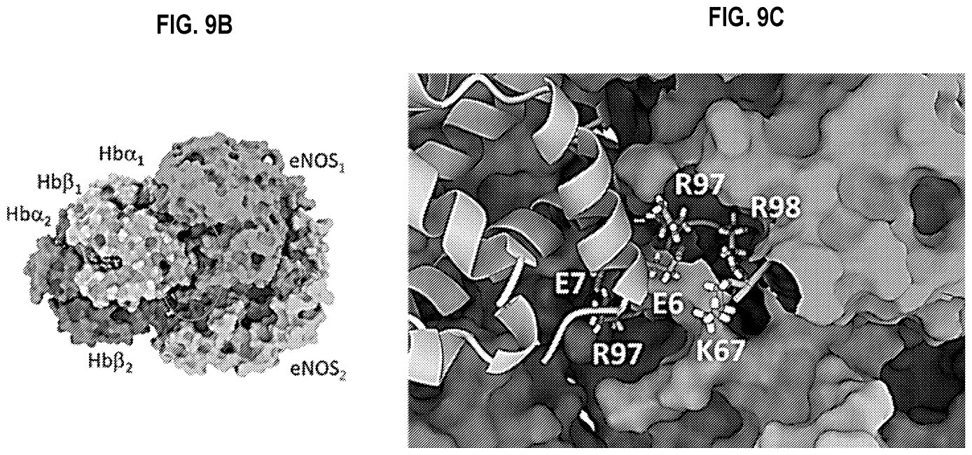

This peptide derives from near the N-terminus of b-globin and is mostly alpha-helical in crystal structures of hemoglobin. The Jpred4 prediction server also predicts mostly an alpha helical structure based just on the amino acid sequence. It interacts with eNOS near the interface between the two subunits of the eNOS homodimer, and has close contacts with six residues on eNOS which are from both subunits. These are LYS67, GLN90, and SER125 on one subunit, and THR95, ARG97, and ARG98 on the other chain. Structural features of each amino acid in Hbb1, and their interactions with eNOS, are described in table 1 and shown in FIGS. 9A-9C.

TABLE 1

| | | Near | Structurally | |
|---|---|---|---|---|
| Position | AA | eNOS | important | Comment |
| 4 | T | | X | Probably important electrostatically, could be |
| | | | | replaced with S |
| 5 | P | X | X | Important for structure |
| 6 | E | X | X | Critical residue, may form 2 salt bridges with |
| | | | | eNOS in complex |
| 7 | E | X | X | Important residues, may also form salt bridges in |
| | | | | some conformations |
| 8 | K | | X | May be important electrostatically |
| 9 | S | X | X | Near R97 on eNOS |
| 10 | A | X | X | Near Q90 on eNOS |
| 11 | V | | x | May be replaced by hydrophobic AA |
| 12 | T | | | May be replaced by S or hydrophobic AA |
| 13 | A | x | | Near eNOS, may be important in some |
| | | | | conformations |
| 14 | L | | | Interacts closely with HBB2 |
| 15 | W | | x | May be important structurally |
| 16 | G | | | Sidechain substitutions would not interact with |
| | | | | eNOS, may be mutated |
| 17 | K | X | X | Important for anchoring end of peptide to S125 on |
| | | | | eNOS |

The wild-type sequence for Hbb1 accommodates the requirements of its local structure in hemoglobin. When it is isolated, some positions can be modified to improve its binding to eNOS or improve its innate structural stability. For example, L14 only has close contact with Hbb2, so its mutation would be expected to have little impact on the ability to bind to eNOS, and such mutations may improve the usefulness of Hbb1 as a mimetic peptide. Based on this analysis, the canonical sequence for HBB1 could be substituted at some positions as follows:

Hbb1-
(SEQ ID NO: 4)
TPEEKSAVTALWGK
Variations-
(SEQ ID NO: 1)
X₁PEEX₂SAX₃X₄AX₅WX₆K

wherein X₁ is T or S, Xₐ is K or R, X₃ is a hydrophobic amino acid, X₄ is T, 5, or a hydrophobic amino acid, X₅ is a hydrophobic amino acid, and X₆ is any amino acid.

### Hbb-Peptide-2

This peptide is spatially near Hbb1. It does not have a canonical alpha or beta secondary structure, and the Jpred4 prediction server does not predict a canonical secondary structure. However, it has a well-defined structure in hemoglobin that links two alpha helices in b-globin. Without being bound by theory, maintaining this structure likely is important for its interactions with eNOS. It has close contacts with seven residues on eNOS, and like Hbb1 it interacts with both eNOS subunits. These are GLN9, ASP91, GLN122, ER125, GLN126, and GLU347 on one chain and PR096 on the second eNOS chain. In the model complex, H4bb2 fits into a deep depression on the surface of eNOS, with the side chain of K120 projecting far into the pocket.

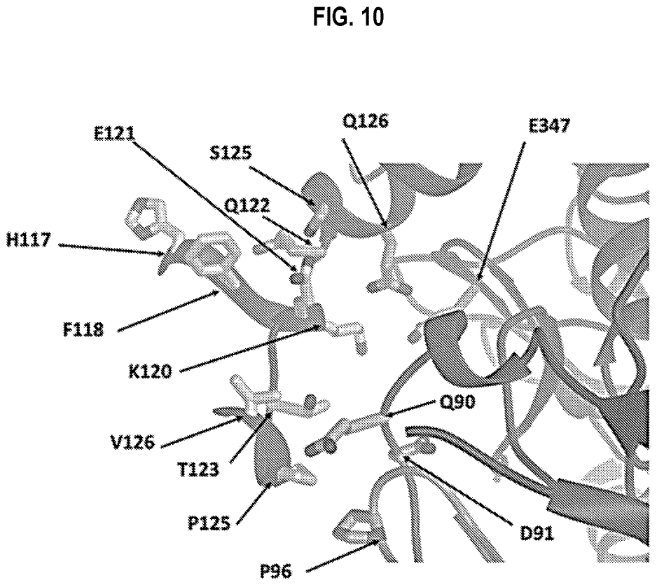

Structural features of each amino acid in Hbb2, and their interactions with eNOS, are described in Table 2 and shown in FIG. 10.

TABLE 2

| | | Near | Structurally | |
|---|---|---|---|---|
| Position | AA | eNOS | important | Comment |
| 117 | H | X | X | Near GLN 122 on eNOS |
| 118 | F | X | x | May be important for correct fold |
| 119 | G | | | Not near eNOS, must be small for binding to |
| | | | | alpha-globin |
| 120 | K | X | X | Projects deep into pocket on eNOS and interacts |
| | | | | with GLU347 |
| 121 | E | X | X | Near S125 on eNOS |
| 122 | F | X | | Only backbone interaction with eNOS, important |
| | | | | for stabilizing b-globin |
| 123 | T | X | | May interact with backbone of GLN90 on eNOS |
| 124 | P | | X | May be important for correct fold |
| 125 | P | X | X | Interacts with PRO96, GLN90, ASP91 on eNOS |
| 126 | V | X | X | Near GLN90 on eNOS, |

Based on a similar analysis to that done for Hbb1, the canonical sequence for Hbb2 can be substituted at some positions as follows:

Hbb2-
(SEQ ID NO: 5)
HFGKEFTPPV
Variations-
(SEQ ID NO: 2)
HFX₇KEX₈X₉PPV

wherein X₇ is any amino acid, X₈ is any amino acid, and X₉ is T or S.

### Hbb-Peptide-3

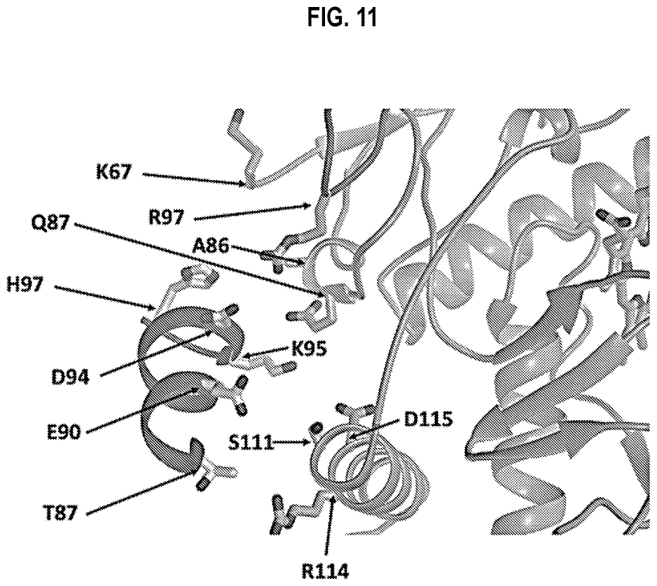

This peptide is a somewhat distorted alpha-helix in b-globin, and there was not an identification of a tendency to form an alpha-helix by the Jpred4 prediction server. Like the other two mimetic peptides, Hbb3 interacts with both subunits of the eNOS homodimer, with close contacts to the 7 amino acids ARG97, LYS67, ALA86, GLN87, SER111, ARG114, and ASP115. See FIG. 11.

TABLE 3

| | | Near | Structurally | |
|---|---|---|---|---|
| Position | AA | eNOS | important | Comment |
| 87 | T | X | | Near R114 on eNOS |
| 88 | L | | | Sidechain substitutions would not interact with eNOS, |
| | | | | may be mutated |
| 89 | S | | | Interacts with C-terminal region of beta-globin |
| 90 | E | X | X | Near S111 on eNOS, may also interact with R114 in |
| | | | | some conformations |
| 91 | L | | X | Sidechain substitutions would not interact with eNOS, |
| | | | | may be mutated |
| 92 | H | | X | Binds to heme iron, may be mutated |
| 93 | C | | | Sidechain substitutions would not interact with eNOS, |
| | | | | binds to C-terminal region of beta-globin, may be |
| | | | | mutated |
| 94 | D | X | X | Near R97 on eNOS |
| 95 | K | X | X | Near Q87 on eNOS |
| 96 | L | | | Near heme, sidechain substitutions would not interact |
| | | | | with eNOS, may be mutated |
| 97 | H | X | X | Near A86 on eNOS, possible interaction with Q87, |
| | | | | could anchor this end of structure, interacts with |
| | | | | alpha-globin |

Based on a similar analysis to that done for Hbb1, the canonical sequence for HBB3 could be substituted at some positions as follows:

Hbb3-
(SEQ ID NO: 6)
TLSELHCDKLH
Variations-
(SEQ ID NO: 3)
TX₁₀X₁₁EX₁₂X₁₃X₁₄DKX₁₅H

wherein X₁₀ is any amino acid, X₁₁ is T or S, X₁₂ is any amino acid, X₁₃ is a polar or uncharged amino acid, X₁₄ is C, M or T, and X₁₅ is any amino acid.

### Additional Peptide Constructs Based Upon Hbb1, Hbb2, and Hbb3.

The C-terminus of Hbb1 is roughly 10A from the N-terminus of Hbb2 in the structure of b-globin and these two peptides could be connected by a short heterologous peptide linker of 3 or more amino acids. The flexible heterologous linker peptide (GGGGS, SEQ ID NO: 48)ₙ with values for n=1, 2, or 3, between Hbb1 and Hbb2. This construct, Hbb1-2, provides a unique mimetic peptide that can provide improved binding free energy with eNOS, requiring a lower dose, and greater specificity to the eNOS target since it would require more specific binding interactions. Note that there are additional constraints on the sequence variability of each component of Hbb1-2 since they have some close contacts with each other in the beta-globin structure.

Similarly, there is only a 20A gap between the C-terminus of Hbb3 and the N-terminus of Hbb1, and a similar construct could be made with copy numbers of n=2, 3, or 4 for the heterologous peptide linker. This peptide is recombinant, as the Hbb1 and Hbb3 sequences are derived from two different beta-globin subunits in the hemoglobin tetramer.

### Example 7

### Testing in Omental Arteries

Collection of omental arteries: Human omental tissue was collected from patients during clinically indicated abdominal operations at the NIH Clinical Center. All patients provided informed consent for tissue procurement on IRB-approved protocol 13-C-0176 (NCT01915225). Tissue removed by the surgeon was immediately submerged in cold 4° C. Krebs-HEPES (KH) buffer (pH 7.4) and kept on ice during dissection and artery isolation. Small arteries were dissected away from the tissue in cold Krebs-HEPES (KH) buffer (Boston BioProducts).

Identification of beta globin mimetic peptides: Tetrameric hemoglobin was docked with dimer eNOS oxygenase domain using molecular docking algorithms (HADDOCK2). Key interfaces between beta globin and eNOS were identified based on predicted hydrophobic interactions, hydrogen bonding, and/or charge complementarity. Three peptide sequences ((Hbb1 (SEQ ID NO: 4), Hbb2 (SEQ ID NO: 5) and Hbb3 (SEQ ID NO: 6)) and corresponding to the regions of beta globin predicted to interact with eNOS were synthesized and appended with an HIV-tat sequence (YGRKKRRQRRR, SEQ ID NO: 14) to confer cell membrane penetration properties. See FIG. 8.

Each mimetic peptide was diluted in KH buffer. Individual isolated arteries were incubated with each peptide at a concentration of 5 μmol/L for 45 minutes at 37° C. before assessing the arterial vasoconstrictor response to phenylephrine.

Isolated artery pressure myography: The pressure myography methodology utilized here for omental arteries is consistent with published protocols for assessing reactivity of mesenteric articles using a DMT pressure myograph. Arteries 100-200 m in diameter were dissected from omental tissue in KH buffer on ice and transferred to the culture myograph wells (DMT-USA CM204). Arteries were cannulated with glass micropipettes and secured on both ends with 10-0 monofilament suture. Arteries were warmed to 37° C. and gradually pressurized to 60 mmHg while perfused with KH buffer and allowed to equilibrate. The culture myograph chambers were then mounted on an inverted microscope at 10× magnification (DMT-USA). Digital video calipers were used to measure in the inner diameter of cannulated arteries (MyoVIEW, DMT-USA) at two positions, and the average of both measurements was recorded.

All arteries developed at least 20% myogenic tone after pressurization, consistent with standards for pressure myography. Arteries were assessed for vasoconstriction response to the alpha-1-adrenoreceptor agonist phenylephrine (PE, Sigma-Aldrich) at successively higher doses ranging from 10⁻⁹ to 10⁻³ molar. Dose response to PE was then re-assessed after incubation with each of the peptides: Hbb-1 (n=2), Hbb-2 (n=1), and Hbb-3 (n=1). Following incubation with each of the peptides, vasoconstriction to PE was tested again following 45 minutes incubation with the NOS inhibitor No-Nitro-L-arginine methyl ester HCl (L-NAME, Sigma-Aldrich) at a concentration of 10⁻⁴ M.

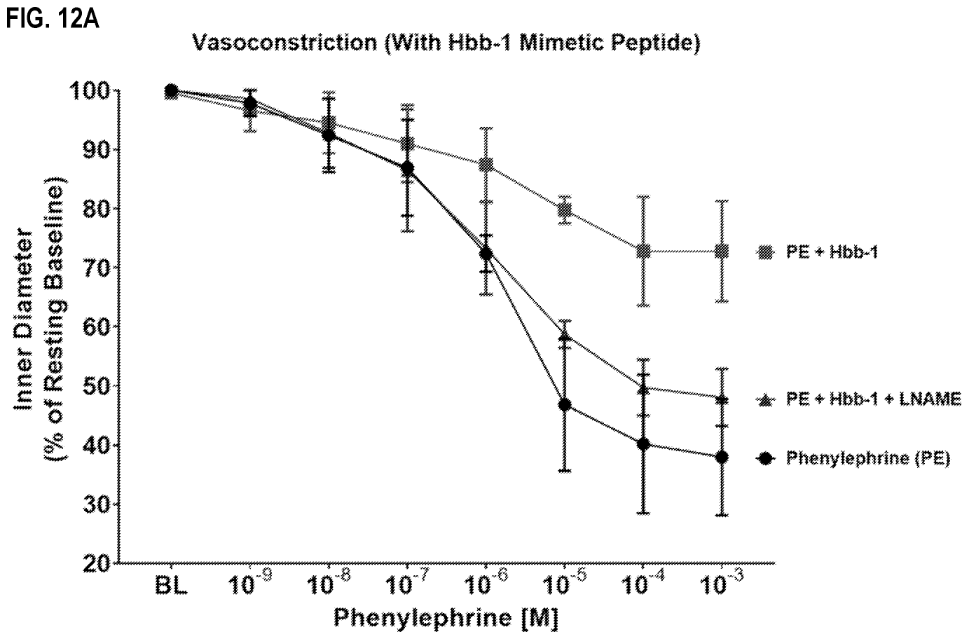

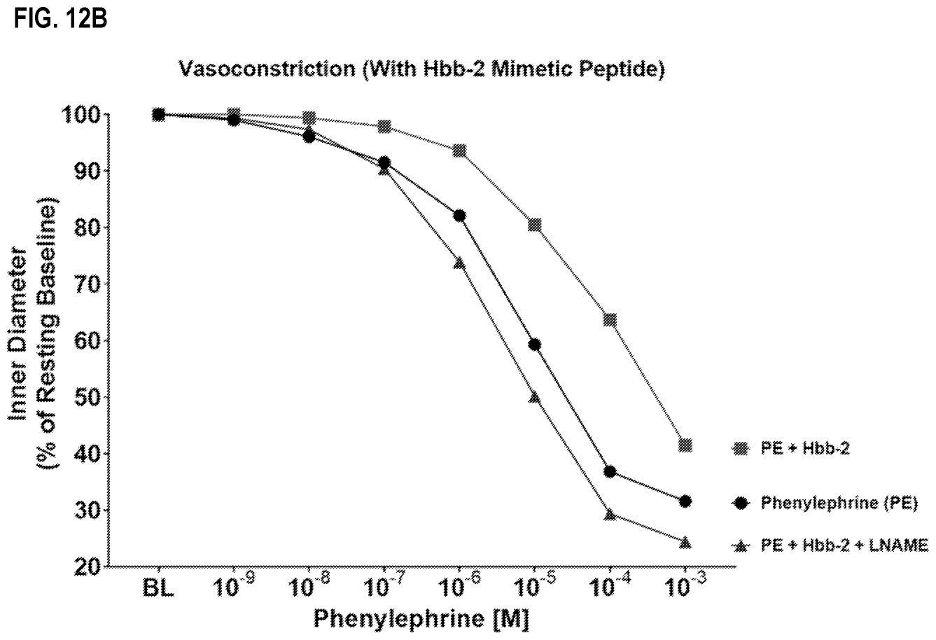

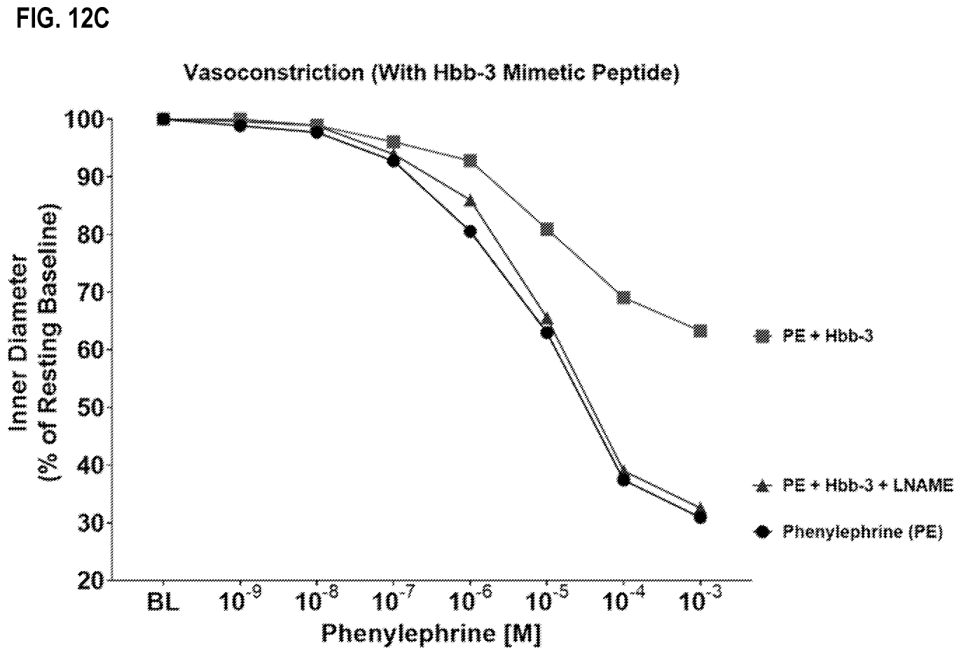

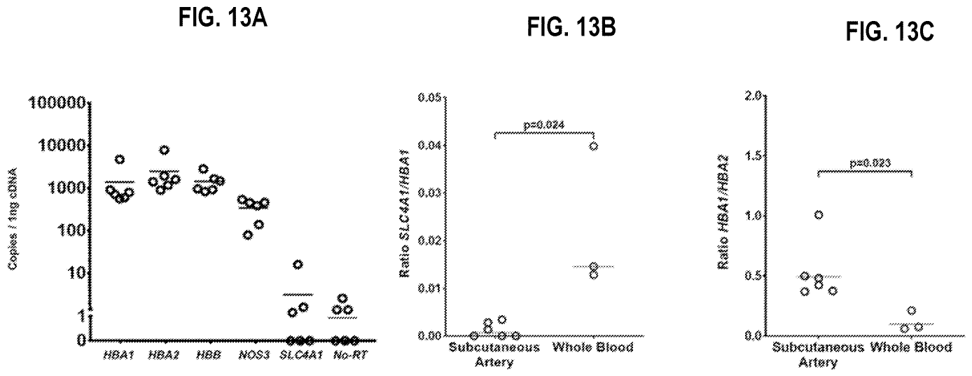

The dose response of each artery to phenylephrine under each condition was measured as inner diameter at baseline and inner diameter at each concentration of phenylephrine. Responses were expressed as a percentage of baseline diameter. The effect of the beta globin mimetic peptides was assessed as the change in the maximal constriction to phenylephrine, which represents the ability of the peptide to inhibit vasoconstriction. The extent to which inhibition of vasoconstriction was dependent on NOS activity was assessed by measuring the inhibition of vasoconstriction in the presence of the NOS inhibitor L-NAME. Results are shown in FIGS. 12A-12C.

### Example 8

Vasodilation in Subjects with Sickle Cell Trait

Healthy individuals were screened for sickle cell trait using hemoglobin electrophoresis of whole blood. Participants underwent a subcutaneous biopsy, adipose tissue was removed, and then resistance arteries 100-300 um in diameter were dissected out of the adipose tissue. Single vessel pressure myography was performed.

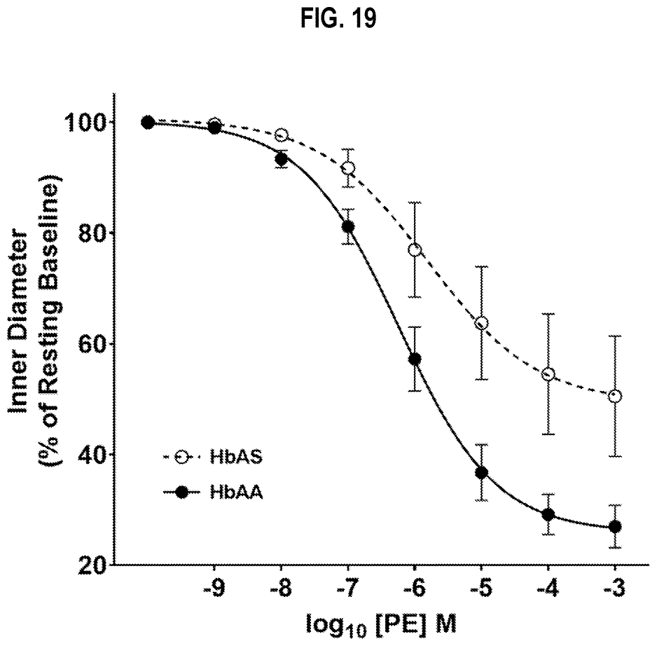

Subcutaneous adipose resistance arteries from donors with sickle cell trait (i.e., heterozygous of the sickle cell mutation [HbAS]) are partially resistant to phenylephrine-induced vasoconstriction. Structural modeling and molecular dynamic simulation suggest that the substitution of glutamic acid with valine and position 6 on human beta globin disrupts salt bridges that would normally stabilize the interaction of hemoglobin with eNOS. The loss of these salt bridges renders hemoglobin less effective at capturing and deoxygenating NO, allowing more NO to escape and signal vasodilation. This is evident as resistance to vasoconstriction induced by phenylephrine, see FIG. 19. In FIG. 19, the vasoconstrictive responses to phenylephrine are presented as a percentage of baseline resting diameter at 60 mm Hg pressure. The mean and standard error of the mean are presented for the sickle cell trait group (HbAS, n=4) and the normal controls (HbSS, n=7).

### Example 9

### Additional Modeling

FIGS. 9A, 9B and 9C shows modeling of the hemoglobin eNOS complex identified interactions involving residues E6 and E7 of beta globin and residues R97, R98 of one eNOS polypeptide, and R98 and K67 of the other eNOS polypeptide. The structure highlighted is the amino acids 4-17 of beta globin which comprise the Hbb-1 peptide.

Additional modeling data is provided in FIGS. 9B and 9C. Models of the human eNOS homodimer and Hb heterotetramer were first built from their crystal structures (PDB 2NOS and 6BB5), subjected to short (5 ns) molecular dynamics (MD) simulations, and then assembled into a dimeric eNOS-Hb complex by overlying each monomer on their predicted interaction mode. Residues 1-66 and 481-1203 of the eNOS protein, absent in the crystal, were not modeled. The N-term of the modeled eNOS (at K67) was acetylated to avoid possible electrostatic artifacts; all other termini in the complex were uncapped. In the crystal structure of human eNOS, the unresolved segment 106-121, also rich in prolines, was modeled by threading on the homologous bovine structure (PDB id: 1NSE); the AlphaFold2 model yielded a similar conformation of this segment. Each of the four Hb heme B groups contained one Fe³⁺ ion tetrahedrally coordinated to the porphyrin ring in a planar conformation and kept covalently bonded to the proximal histidine on one side of the plane and to

$$\\ {O}_{2}^{-}$$

on the other to ensure that Hb remained in the relaxed (R) state; the total charge of the heme-O₂ complex was −2 due to two

$$\\ {CO}_{2}^{-}$$

groups. Two BH4 cofactors were accommodated in the dimeric structure of eNOS but BH4 was not explicitly modeled. Deoxygenated heme groups (one per eNOS protein) and one Zn²⁺ ion tetrahedrally coordinated to four cysteine residues (C94 and C99 in each eNOS protein) were included.

Simulations were carried out in the NPT ensemble, at 37° C. and 1 atm, at [KCl]~150 mM and pH 7, in a cubic cell with PBC and particle-mesh Ewald summations, using the all-atom CHARMM (param36) force field. The side length of the simulation box was initially set at ~12.0 nm and filled with ~56,000 TIP3P water molecules, yielding an average density of ~0.993 g/cm3 at 37° C. after equilibration. Assuming Asp- and Glu unprotonated and Arg⁺ and Lys⁺ protonated, the protonation state of His was set so that the total charge of the monomers and the complex were minimized (His⁺ in eNOS and His⁰ in Hb); the complex was neutralized by the addition of six Cl⁻ ions (four in the E6V mutant), and 112 K⁺ and 112 Cl⁻ ions were added to mimic near-physiological electrolyte concentration. The ions were randomly distributed in the water phase after the complex was solvated and overlapping water molecules removed. All bond lengths involving hydrogen atoms were constrained with the SHAKE algorithm, and an integration step of 2 fs was used. The temperature and pressure were maintained with the Hoover thermostat, using a mass of 10³ kcal mol⁻¹ps², and with the Langevin piston method, with mass and collision frequency of 400 amu and 20 ps⁻¹. After standard protocols of heating and equilibration, a productive phase of 30 ns was conducted, and analysis performed over the last 20 ns. Three independent simulations were performed for the WT and mutant eNOS-Hb complex. Soft definitions of H-bond/salt-bridge and hydrophobic/non-polar interactions are based on a distance (δ) criterion between donor and acceptor atoms (δ_{AD}<4 Å) and side-chain carbon atoms (δ_{CC}<4.8), respectively. The strength of an interaction between two residues is a measure of the number and persistence of the interaction throughout the simulation. All structural and dynamic analyses were performed with the CHARMM analysis facility and in-house scripts.

The interactions at the eNOS/Hb interfaces are shown in FIGS. 9B and 9C. On the left is the overall structure of one hemoglobin tetramer with one eNOS oxygenase domain homodimer. On the right, a close-up view of the specific residues that appear critical for Hb/eNOS binding. The Hb/eNOS complex is stabilized by strong H-bond/salt-bridge interactions, with R97 in both eNOS monomers bound to E6 and E7 of the same HbP monomer and R98 interacting transiently with E6. Several proline residues, particularly in eNOS, contribute to these interactions. K67 appears to interact electrostatically with E6V but does not develop a stable association (based on the distance criterion utilized. However, the eNOS model does not include the segment 1-66, which may help stabilize K67 closer to E6 and further stabilize the wild-type interface. Together, these interactions involving the E6 and E7 residues of beta globin appear important for the stabilization and orientation of hemoglobin with eNOS.

### Example 10

### Canine Expression of Hemoglobin

A survey of different laboratory animal species identified that canines express hemoglobin (alpha and beta subunits) in the endothelium of resistance arteries. Canine beta globin also includes a glutamic acid at position 6. Therefore, it as predicted that the human-based Hbb-1 peptide would have activity on canine resistance arteries.

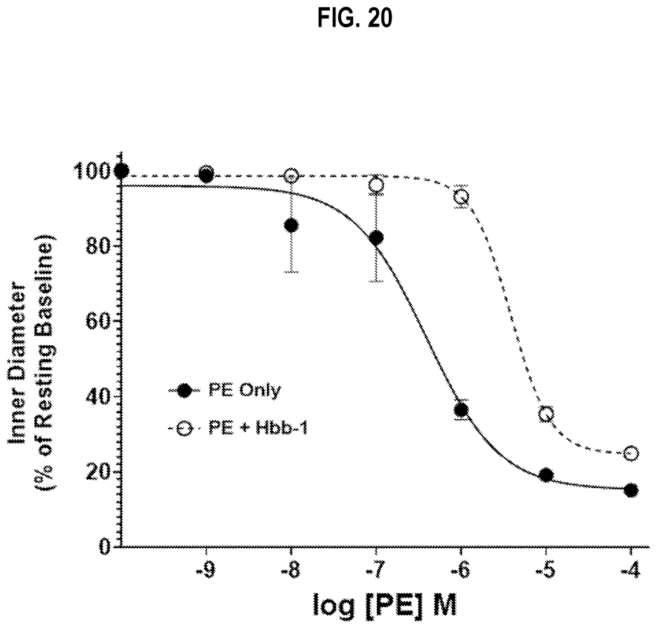

For these studies, omental tissue was removed from a canine laboratory animals soon after euthanasia. Mesenteric arteries 100-300 um in diameter were dissected out of the tissue and cannulated on glass pipettes. The arteries were incubated in a physiologic buffer solution (Krebs-Henseleit) at 37 degrees Celsius and pressured to 60 mm Hg. To assess vasoreactivity, escalating doses of phenylephrine were added to the incubation bath and artery diameter was measured using video microscopy with edge detection and digital calipers. The artery was incubated with 5 uM Hbb-1 peptide (including the tat cell permeability sequence) for 45 minutes. A second phenylephrine dose escalation was applied, and vasoconstriction was measured. The entire experiment was repeated with a second animal donor. In FIG. 20, artery diameter is presented as a percentage of baseline resting diameter at 60 mm Hg of pressure. The means and standard error of the mean were plotted for each condition and phenylephrine dose.

Hbb-1 shifted the vasoconstrictive response to phenylephrine to the right, indicating that in the presence of Hbb-1, the phenylephrine dose must be increased by approximately 100 times to achieve the same degree of vasoconstriction. Thus Hbb-1 can inhibit vasoconstriction in both human and canine arteries.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. An isolated polypeptide comprising a Hbb peptide, wherein the Hbb peptide comprises one of:
a) X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid;
b) HFX₇KEX₈X₉PPV (SEQ ID NO: 2), wherein X₇ is any amino acid, X₈ is any amino acid, and X₉ is T or S; or
c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁ₛ is any amino acid,
and wherein the isolated polypeptide is at most 75 amino acids in length.

2. The isolated polypeptide of claim 1, wherein the Hbb peptide comprises a) X₁PEEX₂SAX₃X₄AX₅WX₆K, (SEQ ID NO: 1, wherein X₁ is T or S, X₂ is K or R, X₃ is a hydrophobic amino acid, X₄ is T, S, or a hydrophobic amino acid, X₅ is a hydrophobic amino acid, and X₆ is any amino acid.

3. The isolated polypeptide of claim 2, wherein the Hbb peptide comprises or consists of TPEEKSAVTALWGK (SEQ ID NO: 4) (hbb1).

4. The isolated polypeptide of claim 2, wherein the Hbb peptide comprises or consists of TPEEKSALTALWGK (SEQ ID NO: 7) or TPEEKSAVTALWLK (SEQ ID NO:8).

5. The isolated polypeptide of claim 1, wherein the Hbb peptide comprises b) HFX₇KEX₈X₉PPV (SEQ ID NO: 2), wherein X₇ is any amino acid, X₅ is any amino acid, and X₉ is T or S.

6. The isolated polypeptide of claim 1, a wherein the Hbb peptide comprises or consists of HFGKEFTPPV (SEQ ID NO: 5) (hbb2).

7. The isolated polypeptide of claim 5, wherein the Hbb peptide comprises or consists of HFGKEKTPPV (SEQ ID NO: 9) or HFGKEFLPPV (SEQ ID NO: 10).

8. The isolated polypeptide of claim 1, wherein the Hbb peptide comprises c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁₅ is any amino acid.

9. The isolated polypeptide of claim 8, wherein the Hbb peptide comprises or consist of TLSELHCDKLH (SEQ ID NO: 6) (hbb3).

10. The isolated polypeptide of claim 8, wherein the Hbb peptide comprises or consists of TLSELYCDKLH (SEQ ID NO: 11) or TLSELHCDKVH (SEQ ID NO: 12).

11. The isolated polypeptide of a comprising no more than 13 consecutive amino acids of SEQ ID NO: 13.

12. The isolated polypeptide of claim 1, comprising a cell penetrating peptide.

13. The isolated polypeptide of claim 12, wherein the cell penetrating peptide comprises or consists of SEQ ID NO: 14 (tat).

14. The isolated polypeptide of 12, comprising a heterologous peptide linker between the cell penetrating peptide and the Hbb peptide.

15. The isolated polypeptide of claim 14, wherein the heterologous peptide linker is 4 or 5 amino acids in length.

16. The isolated polypeptide of claim 14, wherein the heterologous peptide linker comprises glycine and serine.

17. The isolated polypeptide of claim 14, comprising SEQ ID NO: 14 and at least one of SEQ ID NOs: 4, 5, or 6.

18. The isolated polypeptide of claim 17, comprising SEQ ID NO: 4.

19. The isolated polypeptide of claim 18, wherein the cell penetrating peptide is amino terminal to SEQ ID NO: 4.

20. The isolated polypeptide of claim 18, wherein the cell penetrating peptide is carboxy terminal to SEQ ID NO: 4.

21. The isolated polypeptide of claim 1, further comprising a substitution of W by 2-naphthyl-alanine or 1-naphthyl-alanine.

22. A conjugate, comprising the isolated polypeptide of claim 1 conjugated to an HbαX peptide, wherein the HbαX peptide comprises or consists of LSFPTTKTYF (SEQ ID NO: 51); or LSFPTTKTYFPHFDLSHGSA (SEQ ID NO: 52).

23. The conjugate of claim 22, wherein the HbαX peptide is no more than 20 amino acids in length.

24. The conjugate of claim 22, wherein the isolated polypeptide is conjugated to the HbαX peptide by a heterologous linker.

25. The conjugate of claim 24, wherein the heterologous linker is —NH—(CH₂)ₙCO— where n=4-20.

26. The conjugate of claim 24, wherein the heterologous linker is —NH—CH₂—CH₂—(O-CH₂CH₂)ₙOCH₂CO— where n=1-12.

27. The conjugate of claim 24, wherein the heterologous linker is NHCH₂-Aryl-O-Aryl-CO₂H, where Aryl is 1-4 substituted benzene, or 2,5-substituted pyridine, or 2,5-disubstituted pyrazine, or a mixture thereof.

28. The conjugate of claim 22, wherein the heterologous linker comprises two 8-amino-3,6-dioxaoctanoic acid moieties.

29. The conjugate of claim 28, comprising, or consisting of:
a)
YGRKKRRQRRR (SEQ ID NO: 14)-LSFPTTKTYF (SEQ ID NO: 51)-
[heterologous linker]-TPEEKSAVTALWGK (SEQ ID NO: 4);
b)
LSFPTTKTYF (SEQ ID NO: 51-[heterologous linker]-
TPEEKSAVTALWGK (SEQ ID NO: 4)-YGRKKRRQRRR (SEQ ID NO:
14);
c)
YGRKKRRQRRR (SEQ ID NO: 14)-LSFPTTKTYFPHFDLSHGSA
(SEQ ID NO: 52)-[heterologous linker]-TPEEKSAVTALWGK (SEQ ID
NO: 4);
or
d)
LSFPTTKTYFPHFDLSHGSA (SEQ ID NO: 52)--[heterologous linker]-
TPEEKSAVTALWGK (SEQ ID NO: 4)-YGRKKRRQRRR (SEQ ID NO:
14),
and
wherein the heterologous linker is two 8-amino-3,6-dioxaoctanoic acid moieties.

30. An isolated agent comprising:
the isolated polypeptide of claim 1 or the conjugate comprising the isolated polypeptide of claim 1 conjugated to an HbαX peptide, wherein the HbαX peptide comprises or consists of LSFPTTKTYF (SEQ ID NO: 51) or LSFPTTKTYFPHFDLSHGSA (SEQ ID NO: 52), and
a second Hbb peptide, wherein the second Hbb peptide consists of:
a) X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid;
b) HFX₇KEX₈X₉PPV (SEQ ID NO: 2), wherein X₇ is any amino acid, X₈ is any amino acid, and X₉ is T or S; or
c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁₅ is any amino acid.

31. The isolated agent of claim 30, wherein the second Hbb peptide is conjugated to the isolated peptide by a heterologous linker.

32. The isolated agent of claim 31, wherein the heterologous linker is a heterologous peptide linker.

33. An isolated nucleic acid molecule encoding the polypeptide of claim 1.

34-36. (canceled)

37. A pharmaceutical composition comprising an effective amount of:
i) the polypeptide of claim 1,
ii) a conjugate comprising the isolated polypeptide of claim 1 conjugated to an HbαX peptide, wherein the HbαX peptide comprises or consists of LSFPTTKTYF (SEQ ID NO: 51) or LSFPTTKTYFPHFDLSHGSA (SEQ ID NO: 52),
iii) an agent comprising the isolated polypeptide of i) or the conjugate of ii) conjugated to an HbαX peptide, wherein the HbαX peptide comprises or consists of LSFPTTKTYF (SEQ ID NO: 51) or LSFPTTKTYFPHFDLSHGSA (SEQ ID NO: 52), and
a second Hbb peptide, wherein the second Hbb peptide consists of:
a) X₁PEEX₂SAX₃X₄AX₅WX₆K, (SEQ ID NO: 1) wherein X₁ is T or S, X₂ is K or R, X₃ is a hydrophobic amino acid, X₄ is T, S, or a hydrophobic amino acid, X₅ is a hydrophobic amino acid, and X₆ is any amino acid;
b) HFX₇KEX₈X₉PPV (SEQ ID NO: 2), wherein X₇ is any amino acid, X₈ is any amino acid, and X₉ is T or S; or
c) TX₁₀X₁₁EX₁₂X₁₃X₁₄DKX₁₅H (SEQ ID NO: 3), wherein X₁₀ is any amino acid, X₁₁ is T or S, X₁₂ is any amino acid, X₁₃ is a polar or uncharged amino acid, X₁₄ is C, M or T, and X₁₅ is any amino acid; or
iii) the nucleic acid molecule encoding the isolated polypeptide of i), the conjugate of ii) or the agent of iii); and
a pharmaceutically acceptable carrier.

38. A method of increasing vascular nitric oxide bioavailability in a subject in need thereof, comprising administering an effective amount of the pharmaceutical composition of claim 37 to the subject, thereby increasing vascular nitric oxide bioavailability in the subject.

39. A method to inhibit vasoconstriction in a subject, comprising administering to the subject an effective amount of the pharmaceutical composition of claim 37, thereby inhibiting vasoconstriction in the subject.

40-47. (canceled)


SEQUENCE LISTING (53 sequences)

SEQ ID NO: 1 - protein, 14 aa, synthetic construct
  MOD_RES 1: note = X is T or S
  MOD_RES 5: note = X is K or R
  MOD_RES 8: note = X is a hydrophonic amino acid residue
  MOD_RES 9: note = X is T, S, or a hydrophobic amino acid
  MOD_RES 11: note = X is a hydrophobic amino acid
XPEEXSAXXAXWXK

SEQ ID NO: 2 - protein, 10 aa, synthetic construct
  MOD_RES 7: note = X is T or S
HFXKEXXPPV

SEQ ID NO: 3 - protein, 11 aa, synthetic construct
  MOD_RES 3: note = X is T or S
  MOD_RES 6: note = X is a polar or uncharged amino acid
  MOD_RES 7: note = X is C, M, or T
TXXEXXXDKXH

SEQ ID NO: 4 - protein, 14 aa, synthetic construct
TPEEKSAVTALWGK

SEQ ID NO: 5 - protein, 10 aa, synthetic construct
HFGKEFTPPV

SEQ ID NO: 6 - protein, 11 aa, synthetic construct
TLSELHCDKLH

SEQ ID NO: 7 - protein, 14 aa, synthetic construct
TPEEKSALTALWGK

SEQ ID NO: 8 - protein, 14 aa, synthetic construct
TPEEKSAVTALWLK

SEQ ID NO: 9 - protein, 10 aa, synthetic construct
HFGKEKTPPV

SEQ ID NO: 10 - protein, 10 aa, synthetic construct
HFGKEFLPPV

SEQ ID NO: 11 - protein, 11 aa, synthetic construct
TLSELYCDKLH

SEQ ID NO: 12 - protein, 11 aa, synthetic construct
TLSELHCDKVH

SEQ ID NO: 13 - protein, 147 aa, synthetic construct
  MOD_RES 1: note = M is optionally present
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 14 - protein, 11 aa, synthetic construct
YGRKKRRQRRR

SEQ ID NO: 15 - protein, 18 aa, synthetic construct
RQIKIWFQNRRMKWKKGG

SEQ ID NO: 16 - protein, 12 aa, synthetic construct
GRKKRRQRRRPQ

SEQ ID NO: 17 - protein, 11 aa, synthetic construct
YARAAARQARA

SEQ ID NO: 18 - protein, 27 aa, synthetic construct
GWTLNSAGYLLGKINLKALAALAKKIL

SEQ ID NO: 19 - protein, 23 aa, synthetic construct
MGLGLHLLVLAAALQGAKKKRKV

SEQ ID NO: 20 - protein, 29 aa, synthetic construct
CGETCVGGTCNTPGCTCSWPVCTRNGLPV

SEQ ID NO: 21 - protein, 18 aa, synthetic construct
KLALKLALKALKAALKLA

SEQ ID NO: 22 - protein, 27 aa, synthetic construct
GALFLGFLGAAGSTMGAWSQPKKKRKV

SEQ ID NO: 23 - protein, 21 aa, synthetic construct
KETWWETWWTEWSQPKKKRKV

SEQ ID NO: 24 - protein, 15 aa, synthetic construct
SDLWEMMMVSLACQY

SEQ ID NO: 25 - protein, 12 aa, synthetic construct
PLILLRLLRGQF

SEQ ID NO: 26 - protein, 12 aa, synthetic construct
PLIYLRLLRGQF

SEQ ID NO: 27 - protein, 10 aa, synthetic construct
PIEVCMYREP

SEQ ID NO: 28 - protein, 15 aa, synthetic construct
KLWMRWYSPTTRRYG

SEQ ID NO: 29 - protein, 18 aa, synthetic construct
KLALKLALKALKAALKLA

SEQ ID NO: 30 - protein, 18 aa, synthetic construct
LLIILRRRIRKQAHAHSK

SEQ ID NO: 31 - protein, 10 aa, synthetic construct
RRIPNRRPRR

SEQ ID NO: 32 - protein, 9 aa, synthetic construct
RRWWRRWRR

SEQ ID NO: 33 - protein, 16 aa, synthetic construct
RQIKIYFQNRRMKWKK

SEQ ID NO: 34 - protein, 15 aa, synthetic construct
RRRRNRTRRNRRRVR

SEQ ID NO: 35 - protein, 21 aa, synthetic construct
AGYLLGKINLKALAALAKKIL

SEQ ID NO: 36 - protein, 23 aa, synthetic construct
LIRLWSHLIHIWFQNRRLKWKKK

SEQ ID NO: 37 - protein, 22 aa, synthetic construct
MVTVLFRRLRIRRACGPPRVRV

SEQ ID NO: 38 - protein, 16 aa, synthetic construct
YTAIAWVKAFIRKLRK

SEQ ID NO: 39 - protein, 17 aa, synthetic construct
IAWVKAFIRKLRKGPLG

SEQ ID NO: 40 - protein, 13 aa, synthetic construct
CGYGPKKKRKVGG

SEQ ID NO: 41 - protein, 22 aa, synthetic construct
MVRRFLVTLRIRRACGPPRVRV

SEQ ID NO: 42 - protein, 25 aa, synthetic construct
MVKSKIGSWILVLFVSDVGLCKKRP

SEQ ID NO: 43 - protein, 34 aa, synthetic construct
NAATATRGRSAASRPTQRPRAPARSASRPRRPVQ

SEQ ID NO: 44 - protein, 17 aa, synthetic construct
KGEGAAVLLPVLLAAPG

SEQ ID NO: 45 - protein, 26 aa, synthetic construct
DPKGDPKGVTVTVTVTVTGKGDPKPD

SEQ ID NO: 46 - protein, 14 aa, synthetic construct
RRRRRRRRRRRRGC

SEQ ID NO: 47 - protein, 8 aa, synthetic construct
KKKKKKKK

SEQ ID NO: 48 - protein, 5 aa, synthetic construct
GGGGS

SEQ ID NO: 49 - protein, 10 aa, synthetic construct
GGGGSGGGGS

SEQ ID NO: 50 - protein, 15 aa, synthetic construct
GGGGSGGGGSGGGGS

SEQ ID NO: 51 - protein, 10 aa, synthetic construct
LSFPTTKTYF

SEQ ID NO: 52 - protein, 20 aa, synthetic construct
LSFPTTKTYFPHFDLSHGSA

SEQ ID NO: 53 - protein, 17 aa, synthetic construct
TRQARRNRRRRWRERQR